(19) United States
(12) Patent Application Publication
(10) Pub. No.: US 2026/0125465 A1
(43) Pub. Date: May 7, 2026

(54) VHH POLYPEPTIDES THAT BIND TO MESOTHELIN, COMPOSITIONS AND METHODS OF USE THEREOF

(71) Applicants: The Broad Institute, Inc., Cambridge, MA (US); The General Hospital Corporation, Boston, MA (US); Dana-Farber Cancer Institute, Inc., Boston, MA (US)

(72) Inventors: Robert Manguso, Boston, MA (US); Gabriel Griffin, Boston, MA (US); Nelson Knudsen, Cambridge, MA (US); Hans Pope, Cambridge, MA (US)

(73) Assignees: The Broad Institute, Inc., Cambridge, MA (US); The General Hospital Corporation, Boston, MA (US); Dana-Farber Cancer Institute, Inc., Boston, MA (US)

(21) Appl. No.: 19/415,348

(22) Filed: Dec. 10, 2025

Related U.S. Application Data
(63) Continuation of application No. PCT/US2024/034108, filed on Jun. 14, 2024.
(60) Provisional application No. 63/508,189, filed on Jun. 14, 2023.

(51) Int. Cl. C07K 16/28; A61K 35/17; A61K 39/00; A61K 40/11; A61K 40/31; A61K 40/42; A61P 35/00; C07K 14/705; C07K 14/725; C12N 5/0783

(52) U.S. Cl. CPC C07K 16/28; A61K 35/17; A61K 40/11; A61K 40/31; A61K 40/4202; A61P 35/00; C07K 14/7051; C07K 14/70517; C07K 14/70521; C12N 5/0636; A61K 2039/505; A61K 2239/13; A61K 2239/17; A61K 2239/22; C07K 2317/565; C07K 2317/567; C07K 2317/569; C07K 2317/92; C12N 2510/00

(57) ABSTRACT

Single domain VHH polypeptides (antibodies) that bind mesothelin, VHH polypeptide products, methods, cells, pharmaceutical compositions, and kits. The VHH polypeptides may be recombinantly produced and expressed. Provided are also chimeric antigen receptor (CAR) polypeptides comprising the VHH polypeptides and CAR immune effector cells comprising the expressing the same.

### CROSS-REFERENCE TO RELATED APPLICATION

This application is a continuation under 35 U.S.C. § 111(a) of PCT International Patent Application No. PCT/US2024/034108, filed Jun. 14, 2024, designating the United States and published in English, which claims priority to and the benefit of U.S. Provisional Application No. 63/508,189, filed Jun. 14, 2023, the entire contents of each of which are incorporated by reference herein.

### SEQUENCE LISTING

This application contains a Sequence Listing which has been submitted electronically in XML format and is hereby incorporated by reference in its entirety. The Sequence Listing XML file, created on Aug. 13, 2024, is named 167741-051801_PCT_SL.xml and is 4,390,573 bytes in size.

### BACKGROUND

Mesothelin is a glycosylphosphatidylinositol-anchored cell surface protein that has been identified in many different neoplasias, including lung adenocarcinomas, ovarian carcinomas, and hematological malignancies, including acute myeloid leukemia (AML). Chimeric antigen receptor (CAR) T-cell therapy is one method for treating a neoplasia associated with mesothelin. However, to develop effective CAR-T-cell therapies, it is necessary to identify antigen-binding domains suitable for use in targeting the CAR proteins expressed by the CAR-T-cells to neoplasia cells surface-expressing mesothelin.

Accordingly, there is a need for improved antigen binding domains capable of binding a mesothelin antigen.

### SUMMARY

As described below, the disclosure provides single domain VHH polypeptides (nanobodies) that bind mesothelin, VHH polypeptide products, methods of use, cells, pharmaceutical compositions, and kits. The VHH polypeptides may be recombinantly produced and expressed. Provided are also chimeric antigen receptor (CAR) polypeptides comprising the VHH polypeptides and CAR immune effector cells comprising and expressing the same.

In another aspect, the disclosure features a VH-heavy chain only (VHH) polypeptide or an antigen binding portion thereof that specifically binds to mesothelin. The binding protein or the antigen binding portion thereof contains three Complementarity Determining Regions (CDRs): CDR1, CDR2 and CDR3, which are structurally positioned between four camelid VHH framework (FR) regions (FR1-FR4) as follows: FR1-CDR1-FR2-CDR2-FR3-CDR3-FR4. The three CDRs are selected from: CDR1 containing the amino acid sequence GRTFSSYA (SEQ ID NO: 1); CDR2 containing the amino acid sequence ICWSGGNT (SEQ ID NO: 2); and CDR3 containing the amino acid sequence NERTTVLPTGAMDY (SEQ ID NO: 3); CDR1 containing the amino acid sequence GSMFSINA (SEQ ID NO: 4); CDR2 containing the amino acid sequence MTSGGST (SEQ ID NO: 5); and CDR3 containing the amino acid sequence NGGWSLAMDY (SEQ ID NO: 6); CDR1 containing the amino acid sequence GFTLDYYA (SEQ ID NO: 7); CDR2 containing the amino acid sequence ISSSGGST (SEQ ID NO: 8); and CDR3 containing the amino acid sequence AAAGGYGNSSRY (SEQ ID NO: 9); CDR1 containing the amino acid sequence GSIFSVNA (SEQ ID NO: 10); CDR2 containing the amino acid sequence INTGGGST (SEQ ID NO: 11); and CDR3 containing the amino acid sequence AAEDFGNSKTY (SEQ ID NO: 12); CDR1 containing the amino acid sequence GFPYFSCC (SEQ ID NO: 13); CDR2 containing the amino acid sequence ISSDGST (SEQ ID NO: 14); and CDR3 containing the amino acid sequence AASPRQLGPYYAMDY (SEQ ID NO: 15); CDR1 containing the amino acid sequence GFTLDNYA (SEQ ID NO: 16); CDR2 containing the amino acid sequence ISSSGGST (SEQ ID NO: 8); and CDR3 containing the amino acid sequence AGVGDYGRRA (SEQ ID NO: 17); CDR1 containing the amino acid sequence GFTFDDSD (SEQ ID NO: 18); CDR2 containing the amino acid sequence ISSDGST (SEQ ID NO: 14); CDR3 containing the amino acid sequence RTMASRIGC (SEQ ID NO: 19); and any set of CDR1, CDR2, and CDR3 sequences listed in a row of Table 3A.

In another aspect, the disclosure features a method of detecting mesothelin or a fragment thereof in a sample. The method involves contacting the sample with at least one detectably labeled VHH binding protein or an antigen binding fragment thereof that specifically binds to mesothelin or a peptide thereof. The binding protein or the antigen binding fragment thereof contains three Complementarity Determining Regions (CDRs), CDR1, CDR2, and CDR3 structurally positioned between four framework (FR) regions (FR1-FR4) as follows: FR1-CDR1-FR2-CDR2-FR3-CDR3-FR4. The three CDRs are selected from: CDR1 containing the amino acid sequence GRTFSSYA (SEQ ID NO: 1); CDR2 containing the amino acid sequence ICWSGGNT (SEQ ID NO: 2); and CDR3 containing the amino acid sequence NERTTVLPTGAMDY (SEQ ID NO: 3); CDR1 containing the amino acid sequence GSMFSINA (SEQ ID NO: 4); CDR2 containing the amino acid sequence MTSGGST (SEQ ID NO: 5); and CDR3 containing the amino acid sequence NGGWSLAMDY (SEQ ID NO: 6); CDR1 containing the amino acid sequence GFTLDYYA (SEQ ID NO: 7); CDR2 containing the amino acid sequence ISSSGGST (SEQ ID NO: 8); and CDR3 containing the amino acid sequence AAAGGYGNSSRY (SEQ ID NO: 9); CDR1 containing the amino acid sequence GSIFSVNA (SEQ ID NO: 10); CDR2 containing the amino acid sequence INTGGGST (SEQ ID NO: 11); and CDR3 containing the amino acid sequence AAEDFGNSKTY (SEQ ID NO: 12); CDR1 containing the amino acid sequence GFPYFSCC (SEQ ID NO: 13); CDR2 containing the amino acid sequence ISSDGST (SEQ ID NO: 14); and CDR3 containing the amino acid sequence AASPRQLGPYYAMDY (SEQ ID NO: 15); CDR1 containing the amino acid sequence GFTLDNYA (SEQ ID NO: 16); CDR2 containing the amino acid sequence ISSSGGST (SEQ ID NO: 8); and CDR3 containing the amino acid sequence AGVGDYGRRA (SEQ ID NO: 17); CDR1 containing the amino acid sequence GFTFDDSD (SEQ ID NO: 18); CDR2 containing the amino acid sequence ISSDGST (SEQ ID NO: 14); and CDR3 containing the amino acid sequence RTMASRIGC (SEQ ID NO: 19); and any set of CDR1, CDR2, and CDR3 sequences listed in a row of Table 3A. Also, i) FR1 contains the following amino acid sequence:
X₁X₂QLVESGGGX₃VQX₄GX₅X₆LRLX₇CX₈AS (SEQ ID NO: 20), where X₁ is E, D, or Q;
X₂ is V or L; X₃ is L or S; X₄ is A or P; X₅ is E or G; X₆ is S or T; X₇ is S or T, and X₈ is A, T, or V; ii) FR2 contains the following amino acid sequence:
XₐX_{b}X₁₃X₁₄RQX₁₅X₁₆X₁₇X₁₈X₁₉X₂₀X₂₁X₂₂VX₂₃X_{c}, where Xₐ is I, M, or V; X_{b} is A, G, or S;
X₁₃ is W or Y; X₁₄ is F, H, or Y; X₁₅ is A, I, or X; X₁₆ is P or S; X₁₇ is G or T; X₁₈ is D, K, or E; X₁₉ is D, E, or Q; X₂₀ is C or R; X₂₁ is E or N; X₂₂ is F, G, L, or P; X₂₃ is S or A; and X_{c} is A, S, T, or Y; iii) FR3 contains the following amino acid sequence:
X_{d}YAXₑX_{f}VX_{g}GRFTX₂₄SX₂₅X₂₆X₂₇AX₂₈X₂₉X₃₀X₃₁X₃₂LQMX₃₃SLKX₃₄EDTX₃₅X₃₆YX₃₇C (SEQ ID NO: 21), where X_{d} is F, H, I, N, or Y; Xₑ is A or D; X_{f} is S or T; X_{g} is K or R; X₂₄ is I or V; X₂₅ is G, K, R, Q, or W; X₂₆ is D or G; X₂₇ is N or T; X₂₈ is K, N, or R; X₂₉ is N, H, or Q; X₀ is T or S; X₃₁ is V or L; X₃₂ is F or Y; X₃₃ is N, S, or T; X₃₄ is P or S; X₃₅ is A or G;
X₃₆ is I or V; and X₃₇ is F, I, S, or Y; and iv) FR4 contains the following amino acid sequence: X₄₀GQGTX₄₁VTVX₄₂X₄₃ (SEQ ID NO: 22), where X₄₀ is M or W; X₄₁ is L or S; X₄₂ is null or S; and X₄₃ is null or S. The method further involves measuring the level of binding of the binding protein to mesothelin in the sample relative to a control to detect or identify the presence of mesothelin in the sample.

In another aspect, the disclosure features a chimeric antigen receptor (CAR) containing an amino acid sequence with at least about 95% identity to one of the following sequences.

(SEQ ID NO: 23)
MLRLLLALNLFPSIQVTGGQVQLVESGGGLVQPGGSLRLSCAASGSMFSINAVAWYRQAPGE
QREPVAAMTSGGSTIYADSVKGRFTISRDNAKNTVYLQMSSLKPEDTAVYYCNGGWSLAMDY
WGQGTSVTVSPTPAPTIASQPLSLRPEACRPAAGGAVHTRGLDFACDPSKPFWVLVVVGGVL
ACYSLLVTVAFIIFWVRKRGRKKLLYIFKQPFMRPVQTTQEEDGCSCRFPEEEEGGCELASL
RVKFSRSADAPAYQQGQNQLYNELNLGRREEYDVLDKRRGRDPEMGGKPQRRKNPQEGLYNE
LQKDKMAEAYSEIGMKGERRRGKGHDGLYQGLSTATKDTYDALHMQALPPRAS;
(SEQ ID NO: 24)
MLRLLLALNLFPSIQVTGGQVQLVESGGGLVQPGGSLRLSCAASGSIFSVNAMGYYRQISTK
QRNLVAAINTGGGSTYYADSVKGRFTISWDNANNTLFLQMNSLKSEDTAVYYCAAEDFGNSK
TYWGQGTLVTVSPTPAPTIASQPLSLRPEACRPAAGGAVHTRGLDFACDPSKPFWVLVVVGG
VLACYSLLVTVAFIIFWVRKRGRKKLLYIFKQPFMRPVQTTQEEDGCSCRFPEEEEGGCELA
SLRVKFSRSADAPAYQQGQNQLYNELNLGRREEYDVLDKRRGRDPEMGGKPQRRKNPQEGLY
NELQKDKMAEAYSEIGMKGERRRGKGHDGLYQGLSTATKDTYDALHMQALPPRAS;
(SEQ ID NO: 25)
MLRLLLALNLFPSIQVTGGDVQLVESGGGLVQAGGSLRLSCAASGFPYFSCCMSWHRQAPGK
DRELVSSISSDGSTHYADSVRGRFTISKGNARNTLYLQMNSLKPEDTAVYFCAASPRQLGPY
YAMDYWGQGTSVTVSPTPAPTIASQPLSLRPEACRPAAGGAVHTRGLDFACDPSKPFWVLVV
VGGVLACYSLLVTVAFIIFWVRKRGRKKLLYIFKQPFMRPVQTTQEEDGCSCRFPEEEEGGC
ELASLRVKFSRSADAPAYQQGQNQLYNELNLGRREEYDVLDKRRGRDPEMGGKPQRRKNPQE
GLYNELQKDKMAEAYSEIGMKGERRRGKGHDGLYQGLSTATKDTYDALHMQALPPRAS;
(SEQ ID NO: 26)
MLRLLLALNLFPSIQVTGGQLQLVESGGGLVQPGGSLRLSCAASGFTLDNYAIGWFRQAPGK
EREGVSSISSSGGSTNYADSVKGRFTVSRDNAKHTVYLQMNSLKPEDTAVYYCAGVGDYGRR
AMGQGTSVTVSPTPAPTIASQPLSLRPEACRPAAGGAVHTRGLDFACDPSKPFWVLVVVGGV
LACYSLLVTVAFIIFWVRKRGRKKLLYIFKQPFMRPVQTTQEEDGCSCRFPEEEEGGCELAS
LRVKFSRSADAPAYQQGQNQLYNELNLGRREEYDVLDKRRGRDPEMGGKPQRRKNPQEGLYN
ELQKDKMAEAYSEIGMKGERRRGKGHDGLYQGLSTATKDTYDALHMQALPPRAS;
(SEQ ID NO: 27)
MLRLLLALNLFPSIQVTGGQLQLVESGGGLVQPGGSLRLSCAASGFTLDYYAIGWFRQAPGK
EREGVSYISSSGGSTNYADSVKGRFTISRDNAKNTVYLQMNSLKPEDTGVYSCAAAGGYGNS
SRYWGQGTSVTVSPTPAPTIASQPLSLRPEACRPAAGGAVHTRGLDFACDPSKPFWVLVVVG
GVLACYSLLVTVAFIIFWVRKRGRKKLLYIFKQPFMRPVQTTQEEDGCSCRFPEEEEGGCEL
ASLRVKFSRSADAPAYQQGQNQLYNELNLGRREEYDVLDKRRGRDPEMGGKPQRRKNPQEGL
YNELQKDKMAEAYSEIGMKGERRRGKGHDGLYQGLSTATKDTYDALHMQALPPRAS;
(SEQ ID NO: 28)
MLRLLLALNLFPSIQVTGGEVQLVESGGGSVQAGETLRLSCTASGFTFDDSDMGWYRQAPGD
ECELVSTISSDGSTNYADSVKGRFTISQDNAKNTVYLQMTSLKPEDTAIYSCRTMASRIGCW
GQGTLVTVSPTPAPTIASQPLSLRPEACRPAAGGAVHTRGLDFACDPSKPFWVLVVVGGVLA
CYSLLVTVAFIIFWVRKRGRKKLLYIFKQPFMRPVQTTQEEDGCSCRFPEEEEGGCELASLR
VKFSRSADAPAYQQGQNQLYNELNLGRREEYDVLDKRRGRDPEMGGKPQRRKNPQEGLYNEL
QKDKMAEAYSEIGMKGERRRGKGHDGLYQGLSTATKDTYDALHMQALPPRAS;
and
(SEQ ID NO: 29)
MLRLLLALNLFPSIQVTGGQVQLVESGGGLVQAGGSLRLSCVASGRTFSSYAMGWFRQSPGK
EREFVAAICWSGGNTYYADSVKGRFTISGDNAKNSVYLQMNSLKPEDTAVYICNERTTVLPT
GAMDYWGQGTSVTVSSPTPAPTIASQPLSLRPEACRPAAGGAVHTRGLDFACDPSKPFWVLV
VVGGVLACYSLLVTVAFIIFWVRKRGRKKLLYIFKQPFMRPVQTTQEEDGCSCRFPEEEEGG
CELASLRVKFSRSADAPAYQQGQNQLYNELNLGRREEYDVLDKRRGRDPEMGGKPQRRKNPQ
EGLYNELQKDKMAEAYSEIGMKGERRRGKGHDGLYQGLSTATKDTYDALHMQALPPRAS.

In another aspect, the disclosure features a VH-heavy chain only (VHH) polypeptide or an antigen binding portion thereof that specifically binds to mesothelin. The binding protein or the antigen binding portion thereof contains an amino acid sequence with at least about 95% identity to one of the following sequences:

(SEQ ID NO: 23)
MLRLLLALNLFPSIQVTGGQVQLVESGGGLVQPGGSLRLSCAASGSMFSINAVAWYRQAPGE
QREPVAAMTSGGSTIYADSVKGRFTISRDNAKNTVYLQMSSLKPEDTAVYYCNGGWSLAMDY
WGQGTSVTVSPTPAPTIASQPLSLRPEACRPAAGGAVHTRGLDFACDPSKPFWVLVVVGGVL
ACYSLLVTVAFIIFWVRKRGRKKLLYIFKQPFMRPVQTTQEEDGCSCRFPEEEEGGCELASL
RVKFSRSADAPAYQQGQNQLYNELNLGRREEYDVLDKRRGRDPEMGGKPQRRKNPQEGLYNE
LQKDKMAEAYSEIGMKGERRRGKGHDGLYQGLSTATKDTYDALHMQALPPRAS;
(SEQ ID NO: 24)
MLRLLLALNLFPSIQVTGGQVQLVESGGGLVQPGGSLRLSCAASGSIFSVNAMGYYRQISTK
QRNLVAAINTGGGSTYYADSVKGRFTISWDNANNTLFLQMNSLKSEDTAVYYCAAEDFGNSK
TYWGQGTLVTVSPTPAPTIASQPLSLRPEACRPAAGGAVHTRGLDFACDPSKPFWVLVVVGG
VLACYSLLVTVAFIIFWVRKRGRKKLLYIFKQPFMRPVQTTQEEDGCSCRFPEEEEGGCELA
SLRVKFSRSADAPAYQQGQNQLYNELNLGRREEYDVLDKRRGRDPEMGGKPQRRKNPQEGLY
NELQKDKMAEAYSEIGMKGERRRGKGHDGLYQGLSTATKDTYDALHMQALPPRAS;
(SEQ ID NO: 25)
MLRLLLALNLFPSIQVTGGDVQLVESGGGLVQAGGSLRLSCAASGFPYFSCCMSWHRQAPGK
DRELVSSISSDGSTHYADSVRGRFTISKGNARNTLYLQMNSLKPEDTAVYFCAASPRQLGPY
YAMDYWGQGTSVTVSPTPAPTIASQPLSLRPEACRPAAGGAVHTRGLDFACDPSKPFWVLVV
VGGVLACYSLLVTVAFIIFWVRKRGRKKLLYIFKQPFMRPVQTTQEEDGCSCRFPEEEEGGC
ELASLRVKFSRSADAPAYQQGQNQLYNELNLGRREEYDVLDKRRGRDPEMGGKPQRRKNPQE
GLYNELQKDKMAEAYSEIGMKGERRRGKGHDGLYQGLSTATKDTYDALHMQALPPRAS;
(SEQ ID NO: 26)
MLRLLLALNLFPSIQVTGGQLQLVESGGGLVQPGGSLRLSCAASGFTLDNYAIGWFRQAPGK
EREGVSSISSSGGSTNYADSVKGRFTVSRDNAKHTVYLQMNSLKPEDTAVYYCAGVGDYGRR
AMGQGTSVTVSPTPAPTIASQPLSLRPEACRPAAGGAVHTRGLDFACDPSKPFWVLVVVGGV
LACYSLLVTVAFIIFWVRKRGRKKLLYIFKQPFMRPVQTTQEEDGCSCRFPEEEEGGCELAS
LRVKFSRSADAPAYQQGQNQLYNELNLGRREEYDVLDKRRGRDPEMGGKPQRRKNPQEGLYN
ELQKDKMAEAYSEIGMKGERRRGKGHDGLYQGLSTATKDTYDALHMQALPPRAS;
(SEQ ID NO: 27)
MLRLLLALNLFPSIQVTGGQLQLVESGGGLVQPGGSLRLSCAASGFTLDYYAIGWFRQAPGK
EREGVSYISSSGGSTNYADSVKGRFTISRDNAKNTVYLQMNSLKPEDTGVYSCAAAGGYGNS
SRYWGQGTSVTVSPTPAPTIASQPLSLRPEACRPAAGGAVHTRGLDFACDPSKPFWVLVVVG
GVLACYSLLVTVAFIIFWVRKRGRKKLLYIFKQPFMRPVQTTQEEDGCSCRFPEEEEGGCEL
ASLRVKFSRSADAPAYQQGQNQLYNELNLGRREEYDVLDKRRGRDPEMGGKPQRRKNPQEGL
YNELQKDKMAEAYSEIGMKGERRRGKGHDGLYQGLSTATKDTYDALHMQALPPRAS;
(SEQ ID NO: 28)
MLRLLLALNLFPSIQVTGGEVQLVESGGGSVQAGETLRLSCTASGFTFDDSDMGWYRQAPGD
ECELVSTISSDGSTNYADSVKGRFTISQDNAKNTVYLQMTSLKPEDTAIYSCRTMASRIGCW
GQGTLVTVSPTPAPTIASQPLSLRPEACRPAAGGAVHTRGLDFACDPSKPFWVLVVVGGVLA
CYSLLVTVAFIIFWVRKRGRKKLLYIFKQPFMRPVQTTQEEDGCSCRFPEEEEGGCELASLR
VKFSRSADAPAYQQGQNQLYNELNLGRREEYDVLDKRRGRDPEMGGKPQRRKNPQEGLYNEL
QKDKMAEAYSEIGMKGERRRGKGHDGLYQGLSTATKDTYDALHMQALPPRAS;
and
(SEQ ID NO: 29)
MLRLLLALNLFPSIQVTGGQVQLVESGGGLVQAGGSLRLSCVASGRTFSSYAMGWFRQSPGK
EREFVAAICWSGGNTYYADSVKGRFTISGDNAKNSVYLQMNSLKPEDTAVYICNERTTVLPT
GAMDYWGQGTSVTVSSPTPAPTIASQPLSLRPEACRPAAGGAVHTRGLDFACDPSKPFWVLV
VVGGVLACYSLLVTVAFIIFWVRKRGRKKLLYIFKQPFMRPVQTTQEEDGCSCRFPEEEEGG
CELASLRVKFSRSADAPAYQQGQNQLYNELNLGRREEYDVLDKRRGRDPEMGGKPQRRKNPQ
EGLYNELQKDKMAEAYSEIGMKGERRRGKGHDGLYQGLSTATKDTYDALHMQALPPRAS.

In another aspect, the disclosure features a VH-heavy chain only (VHH) polypeptide containing an amino acid sequence having at least 85% sequence identity to a full-length amino acid sequence of any one of Tables 1, 8A to 8G, or 10A to 10M, or a functional fragment thereof, and/or containing a set of Complementarity Determining Regions: CDR1, CDR2, and CDR3, listed in a row of any one of Tables 3A, 8A to 8G, 10A to 10M, or 11. In another aspect, the disclosure features a VH-heavy chain only (VHH) polypeptide containing a full-length amino acid sequence listed in any one of Tables 1, 8A to 8G, or 10A to 10M. In one aspect, the disclosure features a VH-heavy chain only (VHH) polypeptide or an antigen binding portion thereof that specifically binds to mesothelin, where the binding protein or the antigen binding portion thereof contains three Complementarity Determining Regions (CDRs): CDR1, CDR2 and CDR3, which are structurally positioned between four camelid VHH framework (FR) regions (FR1-FR4) as follows: FR1-CDR1-FR2-CDR2-FR3-CDR3-FR4. The three CDRs are selected from: CDR1 containing amino acid sequence SYAMG (SEQ ID NO: 30); CDR2 containing amino acid sequence AICWSGGNTYYADSVKG (SEQ ID NO: 31); and CDR3 containing amino acid sequence RTTVLPTGAMDY (SEQ ID NO: 32); CDR1 containing amino acid sequence INAVA (SEQ ID NO: 33); CDR2 containing amino acid sequence AMTSGGSTIYADSVKG (SEQ ID NO: 34); and CDR3 containing amino acid sequence GWSLAMDY (SEQ ID NO: 35); CDR1 containing amino acid sequence YYAIG (SEQ ID NO: 36); CDR2 containing amino acid sequence YISSSGGSTNYADSVKG (SEQ ID NO: 37); and CDR3 containing amino acid sequence AGGYGNSSRY (SEQ ID NO: 38); CDR1 containing amino acid sequence VNAMG (SEQ ID NO: 39); CDR2 containing amino acid sequence AINTGGGSTYYADSVKG (SEQ ID NO: 40); and CDR3 containing amino acid sequence EDFGNSKTY (SEQ ID NO: 41); CDR1 containing amino acid sequence SCCMS (SEQ ID NO: 42); CDR2 containing amino acid sequence SISSDGSTHYADSVRG (SEQ ID NO: 43); and CDR3 containing amino acid sequence SPRQLGPYYAMDY (SEQ ID NO: 44); CDR1 containing amino acid sequence NYAIG (SEQ ID NO: 45); CDR2 containing amino acid sequence SISSSGGSTNYADSVKG (SEQ ID NO: 46); and CDR3 containing amino acid sequence VGDYGRRA (SEQ ID NO: 47); CDR1 containing amino acid sequence DSDMG (SEQ ID NO: 48); CDR2 containing amino acid sequence TISSDGSTNYADSVKG (SEQ ID NO: 49); and CDR3 containing amino acid sequence MASRIGC (SEQ ID NO: 50); CDR1 containing amino acid sequence YFAIG (SEQ ID NO: 51); CDR2 containing amino acid sequence YISSSGDSTNYADTVKG (SEQ ID NO: 52); and CDR3 containing amino acid sequence AGGYGNSSRY (SEQ ID NO: 38); and CDR1 containing amino acid sequence SYPMG (SEQ ID NO: 53); CDR2 containing amino acid sequence AISWSGGRTFYAASVKG (SEQ ID NO: 54); and CDR3 containing amino acid sequence RTTVLPTGAMDY (SEQ ID NO: 32).

In another aspect, the disclosure features a polypeptide that specifically binds to mesothelin, where the polypeptide or a mesothelin-binding portion thereof has at least 85% amino acid sequence identity to a sequence selected from one or more of:

(SEQ ID NO: 55)
QVQLVESGGGLVQAGGSLRLSCVASGRTFSSYAMGWFRQSPGKEREFVA
AICWSGGNTYYADSVKGRFTISGDNAKNSVYLQMNSLKPEDTAVYICNE
RTTVLPTGAMDYWGQGTSVTVSS;
(SEQ ID NO: 56)
QVQLVESGGGLVQPGGSLRLSCAASGSMFSINAVAWYRQAPGEQREPVA
AMTSGGSTIYADSVKGRFTISRDNAKNTVYLQMSSLKPEDTAVYYCNGG
WSLAMDYWGQGTSVTVS;
(SEQ ID NO: 57)
QLQLVESGGGLVQPGGSLRLSCAASGFTLDYYAIGWFRQAPGKEREGVS
YISSSGGSTNYADSVKGRFTISRDNAKNTVYLQMNSLKPEDTGVYSCAA
AGGYGNSSRYWGQGTSVTVS;
(SEQ ID NO: 58)
QVQLVESGGGLVQPGGSLRLSCAASGSIFSVNAMGYYRQISTKQRNLVA
AINTGGGSTYYADSVKGRFTISWDNANNTLFLQMNSLKSEDTAVYYCAA
EDFGNSKTYWGQGTLVTVS;
(SEQ ID NO: 59)
DVQLVESGGGLVQAGGSLRLSCAASGFPYFSCCMSWHRQAPGKDRELVS
SISSDGSTHYADSVRGRFTISKGNARNTLYLQMNSLKPEDTAVYFCAAS
PRQLGPYYAMDYWGQGTSVTVS;
(SEQ ID NO: 60)
QLQLVESGGGLVQPGGSLRLSCAASGFTLDNYAIGWFRQAPGKEREGVS
SISSSGGSTNYADSVKGRFTVSRDNAKHTVYLQMNSLKPEDTAVYYCAG
VGDYGRRAMGQGTSVTVS;
(SEQ ID NO: 61)
EVQLVESGGGSVQAGETLRLSCTASGFTFDDSDMGWYRQAPGDECELVS
TISSDGSTNYADSVKGRFTISQDNAKNTVYLQMTSLKPEDTAIYSCRTM
ASRIGCWGQGTLVTVS;
(SEQ ID NO: 62)
QLQLVESGGGLVQPGGSLRLSCAASGFTLDNYAIGWFRQAPGKEREGVS
SISSSGGSTNYADSVKGRFTVSRDNAKQTVYLQMNSLKPEDTAVYYCAG
VGDYGRRAMGQGTSVTVS;
(SEQ ID NO: 63)
QLQLVESGGGLVQPGGSLRLSCAASGFTLDYFAIGWFRQAPGKEREGVS
YISSSGDSTNYADTVKGRFTISRDNAKNTVYLQMNSLKPEDTGVYSCAA
AGGYGNSSRYWGQGTSVTVS;
(SEQ ID NO: 64)
QLQLVESGGGLVQPGGSLRLTCAASGFTLDNYAIGWFRQAPGKEREGVS
SISSSGGSTNYADSVKGRFTVSRDNAKHTVYLQMNSLKPEDTAVYYCAG
VGDYGRRAMGQGTSVTVS
(SEQ ID NO: 65)
QVQLVESGGGLVQAGGSLRLSCAASGRNFSSYPMGWFRQAPGKEREFVA
AISWSGGRTFYAASVKGRFTISRDTAKNSVYLQMNSLKPEDTAVYICNE
RTTVLPTGAMDYWGQGTSVTVS;
and
(SEQ ID NO: 66)
QVQLVESGGGLVQPGGSLRLSCAASGSMFSINAVAWYRQAPGEQREPVA
AMTSGGSTIYADSVKGRFTISRDNAKNTVYLQMNSLKPEDTAVYYCNGG
WSLAMDYWGQGTSVTVS.

In another aspect, the disclosure features a chimeric antigen receptor polypeptide (CAR) containing the polypeptide of any aspect of the disclosure, or embodiments thereof.

In another aspect, the disclosure features a polynucleotide encoding the polypeptide or the CAR of any aspect of the disclosure, or embodiments thereof.

In another aspect, the disclosure features a vector containing the polynucleotide of any aspect of the disclosure, or embodiments thereof.

In another aspect, the disclosure features a cell containing the polynucleotide or vector of any aspect of the disclosure, or embodiments thereof.

In another aspect, the disclosure features a pharmaceutical composition containing an effective amount of the polypeptide, or a mesothelin binding fragment thereof, the CAR, the polynucleotide, the vector, or the cell of any aspect of the disclosure, or embodiments thereof, and a pharmaceutically acceptable excipient, carrier, or diluent.

In another aspect, the disclosure features a method for treating a subject having a neoplasia, the method involves administering to the subject the pharmaceutical composition of any aspect of the disclosure, or embodiments thereof.

In another aspect, the disclosure features a kit containing the polypeptide, or a mesothelin binding fragment thereof, the CAR, the polynucleotide, the vector, the cell, or the pharmaceutical composition of any aspect of the disclosure, or embodiments thereof, and a container, for use in treating a subject having a neoplasia.

In another aspect, the disclosure features a method of detecting mesothelin or a fragment thereof in a sample. The method involves contacting the sample with at least one detectably labeled VHH binding protein or an antigen binding fragment thereof that specifically binds to mesothelin or a peptide thereof. The binding protein or the antigen binding fragment thereof contains three Complementarity Determining Regions (CDRs), CDR1, CDR2, and CDR3 structurally positioned between four framework (FR) regions (FR1-FR4) as follows: FR1-CDR1-FR2-CDR2-FR3-CDR3-FR4. The three CDRs are selected from: CDR1 containing amino acid sequence SYAMG (SEQ ID NO: 30); CDR2 containing amino acid sequence AICWSGGNTYYADSVKG (SEQ ID NO: 31); and CDR3 containing amino acid sequence RTTVLPTGAMDY (SEQ ID NO: 32); CDR1 containing amino acid sequence INAVA (SEQ ID NO: 33); CDR2 containing amino acid sequence AMTSGGSTIYADSVKG (SEQ ID NO: 34); and CDR3 containing amino acid sequence GWSLAMDY (SEQ ID NO: 35); CDR1 containing amino acid sequence YYAIG (SEQ ID NO: 36); CDR2 containing amino acid sequence YISSSGGSTNYADSVKG (SEQ ID NO: 37); and CDR3 containing amino acid sequence AGGYGNSSRY (SEQ ID NO: 38); CDR1 containing amino acid sequence VNAMG (SEQ ID NO: 39); CDR2 containing amino acid sequence AINTGGGSTYYADSVKG (SEQ ID NO: 40); and CDR3 containing amino acid sequence EDFGNSKTY (SEQ ID NO: 41); CDR1 containing amino acid sequence SCCMS (SEQ ID NO: 42); CDR2 containing amino acid sequence SISSDGSTHYADSVRG (SEQ ID NO: 43); and CDR3 containing amino acid sequence SPRQLGPYYAMDY (SEQ ID NO: 44); CDR1 containing amino acid sequence NYAIG (SEQ ID NO: 45); CDR2 containing amino acid sequence SISSSGGSTNYADSVKG (SEQ ID NO: 46); and CDR3 containing amino acid sequence VGDYGRRA (SEQ ID NO: 47); CDR1 containing amino acid sequence DSDMG (SEQ ID NO: 48); CDR2 containing amino acid sequence TISSDGSTNYADSVKG (SEQ ID NO: 49); and CDR3 containing amino acid sequence MASRIGC (SEQ ID NO: 50); CDR1 containing amino acid sequence YFAIG (SEQ ID NO: 51); CDR2 containing amino acid sequence YISSSGDSTNYADTVKG (SEQ ID NO: 52); and CDR3 containing amino acid sequence AGGYGNSSRY (SEQ ID NO: 38); and CDR1 containing amino acid sequence SYPMG (SEQ ID NO: 53); CDR2 containing amino acid sequence AISWSGGRTFYAASVKG (SEQ ID NO: 54); and CDR3 containing amino acid sequence RTTVLPTGAMDY (SEQ ID NO: 32). Further, i) FR1 contains the following amino acid sequence: X₁X₂QLVESGGGX₃VQX₄GX₅X₆LRLX₇CX₈ASGX₉X₁₀X₁₁X₁₂ (SEQ ID NO: 67), where X₁ is E, D, or Q, X₂ is V or L, X₃ is L or S, X₄ is A or P, X₅ is E or G, X₆ is S or T, X₇ is S or T, X₈ is A, T, or V, X₉ is F, R, or S, X₁₀ is I, M, P, or T, X₁₁ is F, L, or Y, and X₁₂ is D, F, or S, and X₁₅ is D, F, N, or S; ii) FR2 contains the following amino acid sequence: X₁₃X₁₄RQX₁₅X₁₆X₁₇X₁₈X₁₉X₂₀X₂₁X₂₂VX₂₃, where X₁₃ is W or Y, X₁₄ is F, H, or Y, X₁₅ is A, I, or X, X₁₆ is P or S, X₁₇ is G or T, X₁₈ is D, K, or E, X₁₉ is D, E, or Q, X₂₀ is C or R, X₂₁ is E or N, X₂₂ is F, G, L, or P, and X₂₃ is S or A, and X₂₆ is S or A; iii) FR3 contains the following amino acid sequence:
RFTX₂₄SX₂₅X₂₆X₂₇AX₂₈X₂₉X₃₀X₃₁X₃₂LQMX₃₃SLKX₃₄EDTX₃₅X₃₆YX₃₇CX₃₈X₃₉ (SEQ ID NO: 68), where X₂₄ is I or V, X₂₅ is G, K, R, Q, or W, X₂₆ is D or G, X₂₇ is N or T, X₂₈ is K, N, or R, X₂₉ is N, H, or Q, X₃₀ is T or S, X₃₁ is V or L, X₃₂ is F or Y, X₃₃ is N, S, or T, X₃₄ is P or S, X₃₅ is A or G, X₃₆ is I or V, X₃₇ is F, I, S, or Y, X₃₈ is A, N, or R, and X₃₉ is A, E, G, or T, or Y; and FR4 contains the following amino acid sequence:
X₄₀GQGTX₄₁VTVX₄₂X₄₃ (SEQ ID NO: 22), where X₄₀ is M or W, X₄₁ is L or S, X₄₂ is null or S, and X₄₃ is null or S. The method further involves measuring the level of binding of the binding protein to mesothelin in the sample relative to a control to detect or identify the presence of mesothelin in the sample.

In any aspect of the disclosure, or embodiments thereof, i) FR1 contains the following amino acid sequence: X₁X₂QLVESGGGX₃VQX₄GX₅X₆LRLX₇CX₈ASGX₉X₁₀X₁₁X₁₂ (SEQ ID NO: 67), where X₁ is E, D, or Q, X₂ is V or L, X₃ is L or S, X₄ is A or P, X₅ is E or G, X₆ is S or T, X₇ is S or T, X₈ is A, T, or V, X₉ is F, R, or S, X₁₀ is I, M, P, or T, X₁₁ is F, L, or Y, and X₁₂ is D, F, or S, and X₁₅ is D, F, N, or S; ii) FR2 contains the following amino acid sequence: X₁₃X₁₄RQX₁₅X₁₆X₁₇X₁₈X₁₉X₂₀X₂₁X₂₂VX₂₃, where X₁₃ is W or Y, X₁₄ is F, H, or Y, X₁₅ is A, I, or X, X₁₆ is P or S, X₁₇ is G or T, X₁₈ is D, K, or E, X₁₉ is D, E, or Q, X₂₀ is C or R, X₂₁ is E or N, X₂₂ is F, G, L, or P, and X₂₃ is S or A, and X₂₆ is S or A; iii) FR3 contains the following amino acid sequence:
RFTX₂₄SX₂₅X₂₆X₂₇AX₂₈X₂₉X₃₀X₃₁X₃₂LQMX₃₃SLKX₃₄EDTX₃₅X₃₆YX₃₇CX₃₈X₃₉ (SEQ ID NO: 68), where X₂₄ is I or V, X₂₅ is G, K, R, Q, or W, X₂₆ is D or G, X₂₇ is N or T, X₂₈ is K, N, or R, X₂₉ is N, H, or Q, X₃₀ is T or S, X₃₁ is V or L, X₃₂ is F or Y, X₃₃ is N, S, or T, X₃₄ is P or S, X₃₅ is A or G, X₃₆ is I or V, X₃₇ is F, I, S, or Y, X₃₈ is A, N, or R, and X₃₉ is A, E, G, or T, or Y; and FR4 contains the following amino acid sequence:
X₄₀GQGTX₄₁VTVX₄₂X₄₃ (SEQ ID NO: 22), where X₄₀ is M or W, X₄₁ is L or S, X₄₂ is null or S, and X₄₃ is null or S.

In any aspect of the disclosure, or embodiments thereof, the CAR contains in order from N-terminus to C-terminus a CD28 signal peptide, the mesothelin-binding polypeptide (e.g., a VHH polypeptide) of any aspect of the disclosure, or embodiments thereof, a CD8 hinge domain, a CD28 transmembrane domain, a CD28 cytoplasmic domain, and a CD3ζ domain.

In any aspect of the disclosure, or embodiments thereof, the polynucleotide contains a sequence that has at least 85% amino acid sequence identity to a sequence selected from one or more of:

(SEQ ID NO: 69)
CAAGTTCAGCTTGTGGAGTCAGGCGGTGGACTCGTTCAAGCCGGAGGCTCACTGCGGTTGTC
TTGTGTCGCATCTGGTCGAACCTTCTCAAGTTATGCTATGGGGTGGTTCCGCCAGTCTCCTG
GTAAAGAGCGGGAATTTGTCGCAGCAATTTGTTGGTCTGGTGGTAACACTTACTATGCTGAT
TCAGTAAAAGGTCGATTCACCATATCAGGGGATAATGCAAAAAATTCTGTCTATCTCCAAAT
GAACTCTCTGAAGCCTGAAGATACTGCTGTGTATATCTGTAATGAGAGGACTACGGTACTAC
CTACGGGTGCTATGGACTACTGGGGTCAAGGGACCTCAGTCACCGTCTCCTCG;
(SEQ ID NO: 70)
CAAGTTCAGCTTGTCGAGAGTGGTGGAGGATTGGTGCAGCCAGGCGGATCTCTCAGACTTTC
CTGCGCCGCCAGTGGTTCTATGTTTTCCATAAATGCAGTGGCCTGGTACAGGCAAGCACCAG
GCGAGCAAAGGGAACCGGTAGCCGCAATGACCTCTGGAGGTAGTACAATTTACGCTGATAGC
GTCAAGGGACGATTCACAATATCACGAGACAATGCCAAGAACACTGTCTACCTTCAAATGAG
CAGTTTGAAACCTGAGGATACAGCAGTATACTACTGCAATGGAGGATGGTCACTGGCTATGG
ACTACTGGGGTCAAGGAACCTCAGTCACCGTCTCC;
(SEQ ID NO: 71)
CAGCTTCAACTGGTAGAGAGTGGTGGAGGACTTGTTCAGCCTGGAGGGTCCCTTAGACTTAG
TTGCGCTGCCAGTGGGTTCACCCTGGACTACTACGCCATTGGCTGGTTTCGACAGGCCCCAG
GGAAAGAGCGAGAGGGGGTCTCTTACATCAGTAGTTCCGGTGGTTCCACTAACTACGCTGAT
AGTGTGAAGGGGAGGTTTACTATTTCTCGGGATAATGCAAAAAATACCGTGTACCTGCAAAT
GAATAGCCTCAAACCAGAAGACACAGGCGTTTATTCTTGCGCAGCAGCTGGGGGCTATGGTA
ACTCCTCTCGCTACTGGGGTCAAGGAACCTCAGTCACCGTCTCC;
(SEQ ID NO: 72)
CAGGTTCAGCTGGTGGAGTCCGGCGGAGGTCTTGTTCAGCCCGGAGGTTCCCTGCGCCTCTC
TTGTGCAGCTTCTGGTTCCATTTTCTCAGTCAATGCAATGGGCTACTACAGACAAATTTCCA
CAAAGCAGCGCAACTTGGTCGCCGCAATAAATACTGGTGGTGGCTCGACTTATTACGCCGAT
AGTGTCAAAGGACGATTTACTATAAGTTGGGACAACGCAAACAATACTTTGTTCCTCCAAAT
GAATAGCCTCAAATCAGAAGACACTGCCGTCTACTACTGTGCAGCAGAGGACTTTGGTAACT
CGAAAACTTACTGGGGCCAAGGGACTCTGGTCACTGTCTCT;
(SEQ ID NO: 73)
GACGTACAACTTGTGGAATCAGGTGGGGGTCTCGTTCAAGCTGGTGGGTCACTGCGGCTTTC
TTGCGCTGCAAGTGGGTTCCCTTATTTTTCCTGCTGTATGAGCTGGCATCGCCAAGCTCCTG
GAAAAGATCGGGAACTGGTCTCCAGTATTTCATCTGACGGGTCAACACATTACGCTGATAGC
GTCAGGGGGAGGTTTACTATTTCTAAAGGAAATGCAAGAAACACACTCTACCTCCAGATGAA
CAGTCTGAAGCCCGAAGACACCGCCGTGTATTTCTGTGCAGCTTCCCCTAGACAGCTCGGGC
CTTACTATGCTATGGACTACTGGGGTCAAGGAACCTCAGTCACCGTCTCC;
(SEQ ID NO: 74)
CAGCTTCAACTGGTAGAGAGTGGTGGAGGACTTGTTCAGCCTGGAGGGTCCCTTAGACTTAG
TTGCGCTGCCAGTGGGTTCACCCTGGACAACTACGCCATTGGCTGGTTTCGACAGGCCCCAG
GGAAAGAGCGAGAGGGGGTCTCTAGCATCAGTAGTTCCGGTGGTTCCACCAACTATGCTGAT
AGTGTGAAGGGGAGGTTTACTGTTTCTCGGGATAATGCAAAACATACCGTGTACCTGCAAAT
GAATAGCCTCAAACCAGAAGACACAGCCGTCTATTATTGCGCAGGAGTTGGGGACTACGGTA
GAAGGGCGATGGGTCAAGGCACCTCAGTCACCGTCTCC;
(SEQ ID NO: 75)
GAAGTTCAGCTTGTTGAATCAGGCGGCGGAAGTGTCCAAGCTGGCGAAACCCTTCGGCTCTC
CTGCACCGCATCCGGCTTCACATTCGATGATTCAGATATGGGCTGGTACAGGCAGGCTCCTG
GTGACGAATGTGAGCTGGTATCCACAATCTCCAGCGATGGCAGCACAAATTATGCTGACTCT
GTTAAAGGGCGATTTACCATCTCTCAAGATAACGCAAAGAACACCGTGTATCTGCAGATGAC
CTCATTGAAGCCAGAGGATACTGCCATATATTCATGCAGAACAATGGCCTCGAGGATTGGTT
GCTGGGGCCAAGGGACTCTGGTCACTGTCTCT;
(SEQ ID NO: 76)
CAGCTTCAACTGGTAGAGAGTGGTGGAGGACTTGTTCAGCCTGGAGGGTCCCTTAGACTTAG
TTGCGCTGCCAGTGGATTCACCCTGGACAACTACGCCATTGGCTGGTTTCGACAGGCCCCAG
GGAAAGAGCGAGAGGGGGTCTCTAGTATCAGTAGTTCCGGTGGTTCCACCAACTATGCCGAT
AGTGTGAAGGGGAGGTTTACTGTTTCTCGGGATAATGCAAAACAAACCGTGTACCTGCAAAT
GAATAGCCTCAAACCAGAAGACACAGCCGTCTATTATTGCGCAGGAGTTGGGGACTACGGTA
GAAGGGCGATGGGTCAAGGCACCTCAGTCACCGTCTCC;
(SEQ ID NO: 77)
CAGCTTCAACTGGTAGAGAGTGGTGGAGGACTTGTTCAGCCTGGAGGGTCCCTTAGACTTAG
TTGCGCTGCCAGTGGGTTCACCCTGGACTACTTCGCCATTGGCTGGTTTCGACAGGCCCCAG
GGAAAGAGCGAGAGGGGGTCTCTTACATCAGTAGTTCCGGTGATTCCACTAACTACGCTGAT
ACTGTGAAGGGGAGGTTTACTATTTCTCGGGATAATGCAAAAAATACCGTGTACCTGCAAAT
GAATAGCCTCAAACCAGAAGACACAGGCGTTTATTCTTGCGCAGCAGCTGGGGGCTATGGTA
ACTCCTCTCGCTACTGGGGTCAAGGAACCTCAGTCACCGTCTCC;
(SEQ ID NO: 78)
CAGCTTCAACTGGTAGAGAGTGGTGGAGGACTTGTTCAGCCTGGAGGGTCCCTTAGACTTAC
TTGCGCTGCCAGTGGGTTCACCCTGGACAACTACGCCATTGGCTGGTTTCGACAGGCCCCAG
GGAAAGAGCGAGAGGGGGTCTCTAGCATCAGTAGTTCCGGTGGTTCCACCAACTATGCTGAT
AGTGTGAAGGGGAGGTTTACTGTTTCTCGGGATAATGCAAAACATACCGTGTACCTGCAAAT
GAATAGCCTCAAACCAGAAGACACAGCCGTCTATTATTGCGCAGGAGTTGGGGACTACGGTA
GAAGGGCGATGGGTCAAGGCACCTCAGTCACCGTCTCC;
(SEQ ID NO: 75)
GAAGTTCAGCTTGTTGAATCAGGCGGCGGAAGTGTCCAAGCTGGCGAAACCCTTCGGCTCTC
CTGCACCGCATCCGGCTTCACATTCGATGATTCAGATATGGGCTGGTACAGGCAGGCTCCTG
GTGACGAATGTGAGCTGGTATCCACAATCTCCAGCGATGGCAGCACAAATTATGCTGACTCT
GTTAAAGGGCGATTTACCATCTCTCAAGATAACGCAAAGAACACCGTGTATCTGCAGATGAC
CTCATTGAAGCCAGAGGATACTGCCATATATTCATGCAGAACAATGGCCTCGAGGATTGGTT
GCTGGGGCCAAGGGACTCTGGTCACTGTCTCT;
and
(SEQ ID NO: 79)
CAAGTCCAGCTCGTCGAGAGTGGTGGAGGATTGGTGCAGCCAGGCGGATCTCTCAGACTTTC
CTGCGCCGCCAGTGGTTCTATGTTTTCCATAAATGCAGTGGCCTGGTACAGGCAAGCACCAG
GCGAGCAAAGGGAACCGGTAGCCGCAATGACCTCTGGAGGTAGTACAATTTACGCTGATAGC
GTCAAGGGACGATTCACAATATCACGAGACAATGCCAAGAACACTGTCTACCTTCAAATGAA
CAGTTTGAAACCTGAGGATACAGCAGTATACTACTGCAATGGAGGATGGTCACTGGCTATGG
ACTACTGGGGTCAAGGAACCTCAGTCACCGTCTCC.

In any aspect of the disclosure, or embodiments thereof, the vector is an expression vector.

In any aspect of the disclosure, or embodiments thereof, the cell in an immune cell. In embodiments, the immune cell is a T cell. In embodiments, the cell is in vitro, ex vivo, or in vivo.

In any aspect of the disclosure, or embodiments thereof, the subject is a mammal. In embodiments, the mammal is a human.

In any aspect of the disclosure, or embodiments thereof, the sample is selected from biopsy, blood, peripheral blood, serum, plasma, cerebrospinal fluid, urine, saliva, sputum, tears, stool, or synovial fluid.

Compositions and articles of the disclosure were isolated or otherwise manufactured in connection with the examples provided below. Other features and advantages of the various aspects and embodiments of the disclosure will be apparent from the detailed description, and from the claims.

In any aspect of the disclosure, or embodiments thereof, i) FR1 contains the following amino acid sequence: X₁X₂QLVESGGGX₃VQX₄GX₅X₆LRLX₇CX₈AS (SEQ ID NO: 20), where X₁ is E, D, or Q; X₂ is V or L; X₃ is L or S; X₄ is A or P; X₅ is E or G; X₆ is S or T; X₇ is S or T; and X₈ is A, T, or V; ii) FR2 contains the following amino acid sequence:
XₐX_{b}X₁₃X₁₄RQX₁₅X₁₆X₁₇X₁₈X₁₉X₂₀X₂₁X₂₂VX₂₃X_{c}, where Xₐ is I, M, or V; X_{b} is A, G, or S;
X₁₃ is W or Y; X₁₄ is F, H, or Y; X₁₅ is A, I, or X; X₁₆ is P or S; X₁₇ is G or T; X₁₈ is D, K, or E; X₁₉ is D, E, or Q; X₂₀ is C or R; X₂₁ is E or N; X₂₂ is F, G, L, or P; X₂₃ is S or A; and X_{c} is A, S, T, or Y; iii) FR3 contains the following amino acid sequence:
X_{d}YAXₑX_{f}VX_{g}GRFTX₂₄SX₂₅X₂₆X₂₇AX₂₈X₂₉X₃₀X₃₁X₃₂LQMX₃₃SLKX₃₄EDTX₃₅X₃₆YX₃₇C (SEQ ID NO: 21), where X_{d} is F, H, I, N, or Y; Xₑ is A or D; X_{f} is S or T; X_{g} is K or R; X₂₄ is I or V; X₂₅ is G, K, R, Q, or W; X₂₆ is D or G; X₂₇ is N or T; X₂₈ is K, N, or R; X₂₉ is N, H, or Q; X₃₀ is T or S; X₃₁ is V or L; X₃₂ is F or Y; X₃₃ is N, S, or T; X₃₄ is P or S; X₃₅ is A or G; X₃₆ is I or V; and X₃₇ is F, I, S, or Y; and iv) FR4 contains the following amino acid sequence: X₄₀GQGTX₄₁VTVX₄₂X₄₃ (SEQ ID NO: 22), where X₄₀ is M or W; X₄₁ is L or S;
X₄₂ is null or S; and X₄₃ is null or S.

In any aspect of the disclosure, or embodiments thereof, the polypeptide binds a mesothelin antigen with a K_{d} of less than 12 nM.

In any aspect of the disclosure, or embodiments thereof, the polypeptide contains an amino acid sequence with at least about 85% identity to one of the following sequences:

(SEQ ID NO: 23)
MLRLLLALNLFPSIQVTGGQVQLVESGGGLVQPGGSLRLSCAASGSMFSINAVAWYRQAPGE
QREPVAAMTSGGSTIYADSVKGRFTISRDNAKNTVYLQMSSLKPEDTAVYYCNGGWSLAMDY
WGQGTSVTVSPTPAPTIASQPLSLRPEACRPAAGGAVHTRGLDFACDPSKPFWVLVVVGGVL
ACYSLLVTVAFIIFWVRKRGRKKLLYIFKQPFMRPVQTTQEEDGCSCRFPEEEEGGCELASL
RVKFSRSADAPAYQQGQNQLYNELNLGRREEYDVLDKRRGRDPEMGGKPQRRKNPQEGLYNE
LQKDKMAEAYSEIGMKGERRRGKGHDGLYQGLSTATKDTYDALHMQALPPRAS;
(SEQ ID NO: 24)
MLRLLLALNLFPSIQVTGGQVQLVESGGGLVQPGGSLRLSCAASGSIFSVNAMGYYRQISTK
QRNLVAAINTGGGSTYYADSVKGRFTISWDNANNTLFLQMNSLKSEDTAVYYCAAEDFGNSK
TYWGQGTLVTVSPTPAPTIASQPLSLRPEACRPAAGGAVHTRGLDFACDPSKPFWVLVVVGG
VLACYSLLVTVAFIIFWVRKRGRKKLLYIFKQPFMRPVQTTQEEDGCSCRFPEEEEGGCELA
SLRVKFSRSADAPAYQQGQNQLYNELNLGRREEYDVLDKRRGRDPEMGGKPQRRKNPQEGLY
NELQKDKMAEAYSEIGMKGERRRGKGHDGLYQGLSTATKDTYDALHMQALPPRAS;
(SEQ ID NO: 25)
MLRLLLALNLFPSIQVTGGDVQLVESGGGLVQAGGSLRLSCAASGFPYFSCCMSWHRQAPGK
DRELVSSISSDGSTHYADSVRGRFTISKGNARNTLYLQMNSLKPEDTAVYFCAASPRQLGPY
YAMDYWGQGTSVTVSPTPAPTIASQPLSLRPEACRPAAGGAVHTRGLDFACDPSKPFWVLVV
VGGVLACYSLLVTVAFIIFWVRKRGRKKLLYIFKQPFMRPVQTTQEEDGCSCRFPEEEEGGC
ELASLRVKFSRSADAPAYQQGQNQLYNELNLGRREEYDVLDKRRGRDPEMGGKPQRRKNPQE
GLYNELQKDKMAEAYSEIGMKGERRRGKGHDGLYQGLSTATKDTYDALHMQALPPRAS;
(SEQ ID NO: 26)
MLRLLLALNLFPSIQVTGGQLQLVESGGGLVQPGGSLRLSCAASGFTLDNYAIGWFRQAPGK
EREGVSSISSSGGSTNYADSVKGRFTVSRDNAKHTVYLQMNSLKPEDTAVYYCAGVGDYGRR
AMGQGTSVTVSPTPAPTIASQPLSLRPEACRPAAGGAVHTRGLDFACDPSKPFWVLVVVGGV
LACYSLLVTVAFIIFWVRKRGRKKLLYIFKQPFMRPVQTTQEEDGCSCRFPEEEEGGCELAS
LRVKFSRSADAPAYQQGQNQLYNELNLGRREEYDVLDKRRGRDPEMGGKPQRRKNPQEGLYN
ELQKDKMAEAYSEIGMKGERRRGKGHDGLYQGLSTATKDTYDALHMQALPPRAS;
(SEQ ID NO: 27)
MLRLLLALNLFPSIQVTGGQLQLVESGGGLVQPGGSLRLSCAASGFTLDYYAIGWFRQAPGK
EREGVSYISSSGGSTNYADSVKGRFTISRDNAKNTVYLQMNSLKPEDTGVYSCAAAGGYGNS
SRYWGQGTSVTVSPTPAPTIASQPLSLRPEACRPAAGGAVHTRGLDFACDPSKPFWVLVVVG
GVLACYSLLVTVAFIIFWVRKRGRKKLLYIFKQPFMRPVQTTQEEDGCSCRFPEEEEGGCEL
ASLRVKFSRSADAPAYQQGQNQLYNELNLGRREEYDVLDKRRGRDPEMGGKPQRRKNPQEGL
YNELQKDKMAEAYSEIGMKGERRRGKGHDGLYQGLSTATKDTYDALHMQALPPRAS;
(SEQ ID NO: 28)
MLRLLLALNLFPSIQVTGGEVQLVESGGGSVQAGETLRLSCTASGFTFDDSDMGWYRQAPGD
ECELVSTISSDGSTNYADSVKGRFTISQDNAKNTVYLQMTSLKPEDTAIYSCRTMASRIGCW
GQGTLVTVSPTPAPTIASQPLSLRPEACRPAAGGAVHTRGLDFACDPSKPFWVLVVVGGVLA
CYSLLVTVAFIIFWVRKRGRKKLLYIFKQPFMRPVQTTQEEDGCSCRFPEEEEGGCELASLR
VKFSRSADAPAYQQGQNQLYNELNLGRREEYDVLDKRRGRDPEMGGKPQRRKNPQEGLYNEL
QKDKMAEAYSEIGMKGERRRGKGHDGLYQGLSTATKDTYDALHMQALPPRAS;
and
(SEQ ID NO: 29)
MLRLLLALNLFPSIQVTGGQVQLVESGGGLVQAGGSLRLSCVASGRTFSSYAMGWFRQSPGK
EREFVAAICWSGGNTYYADSVKGRFTISGDNAKNSVYLQMNSLKPEDTAVYICNERTTVLPT
GAMDYWGQGTSVTVSSPTPAPTIASQPLSLRPEACRPAAGGAVHTRGLDFACDPSKPFWVLV
VVGGVLACYSLLVTVAFIIFWVRKRGRKKLLYIFKQPFMRPVQTTQEEDGCSCRFPEEEEGG
CELASLRVKFSRSADAPAYQQGQNQLYNELNLGRREEYDVLDKRRGRDPEMGGKPQRRKNPQ
EGLYNELQKDKMAEAYSEIGMKGERRRGKGHDGLYQGLSTATKDTYDALHMQALPPRAS.

In any aspect of the disclosure, or embodiments thereof, polynucleotide contains a sequence that has at least 85% identity to a sequence selected from one or more of:

(SEQ ID NO: 80)
ATGCTCAGGCTGCTCTTGGCTCTCAACTTATTCCCTTCAATTCAAGTAACAGGAGGGCAAGT
TCAGCTTGTCGAGAGTGGTGGAGGATTGGTGCAGCCAGGCGGATCTCTCAGACTTTCCTGCG
CCGCCAGTGGTTCTATGTTTTCCATAAATGCAGTGGCCTGGTACAGGCAAGCACCAGGCGAG
CAAAGGGAACCGGTAGCCGCAATGACCTCTGGAGGTAGTACAATTTACGCTGATAGCGTCAA
GGGACGATTCACAATATCACGAGACAATGCCAAGAACACTGTCTACCTTCAAATGAGCAGTT
TGAAACCTGAGGATACAGCAGTATACTACTGCAATGGAGGATGGTCACTGGCTATGGACTAC
TGGGGTCAAGGAACCTCAGTCACCGTCTCCCCGACGCCAGCGCCAACTATTGCGAGTCAGCC
TCTCAGTCTGCGACCTGAGGCTTGTCGACCAGCAGCCGGAGGCGCAGTGCACACGAGGGGGC
TGGACTTCGCCTGTGATCCTTCTAAGCCCTTTTGGGTGCTGGTGGTGGTTGGTGGAGTCCTG
GCTTGCTATAGCTTGCTAGTAACAGTGGCCTTTATTATTTTCTGGGTGAGGAAACGGGGCAG
AAAGAAACTCCTGTATATATTCAAACAACCATTTATGAGACCAGTACAAACTACTCAAGAGG
AAGATGGCTGTAGCTGCCGATTTCCAGAAGAAGAAGAAGGAGGATGTGAACTGGCTAGCCTG
AGAGTGAAGTTCAGCAGGAGCGCAGACGCCCCCGCGTACCAGCAGGGCCAGAACCAGCTCTA
TAACGAGCTCAATCTAGGACGAAGAGAGGAGTACGATGTTTTGGACAAGAGACGTGGCCGGG
ACCCTGAGATGGGGGGAAAGCCGCAGAGAAGGAAGAACCCTCAGGAAGGCCTGTACAATGAA
CTGCAGAAAGATAAGATGGCGGAGGCCTACAGTGAGATTGGGATGAAAGGCGAGCGCCGGAG
GGGCAAGGGGCACGATGGCCTTTACCAGGGTCTCAGTACAGCCACCAAGGACACCTACGACG
CCCTTCACATGCAGGCCCTGCCCCCTCGCGCTAGC;
(SEQ ID NO: 81)
ATGCTCAGGCTGCTCTTGGCTCTCAACTTATTCCCTTCAATTCAAGTAACAGGAGGGCAGGT
TCAGCTGGTGGAGTCCGGCGGAGGTCTTGTTCAGCCCGGAGGTTCCCTGCGCCTCTCTTGTG
CAGCTTCTGGTTCCATTTTCTCAGTCAATGCAATGGGCTACTACAGACAAATTTCCACAAAG
CAGCGCAACTTGGTCGCCGCAATAAATACTGGTGGTGGCTCGACTTATTACGCCGATAGTGT
CAAAGGACGATTTACTATAAGTTGGGACAACGCAAACAATACTTTGTTCCTCCAAATGAATA
GCCTCAAATCAGAAGACACTGCCGTCTACTACTGTGCAGCAGAGGACTTTGGTAACTCGAAA
ACTTACTGGGGCCAAGGGACTCTGGTCACTGTCTCTCCGACGCCAGCGCCAACTATTGCGAG
TCAGCCTCTCAGTCTGCGACCTGAGGCTTGTCGACCAGCAGCCGGAGGCGCAGTGCACACGA
GGGGGCTGGACTTCGCCTGTGATCCTTCTAAGCCCTTTTGGGTGCTGGTGGTGGTTGGTGGA
GTCCTGGCTTGCTATAGCTTGCTAGTAACAGTGGCCTTTATTATTTTCTGGGTGAGGAAACG
GGGCAGAAAGAAACTCCTGTATATATTCAAACAACCATTTATGAGACCAGTACAAACTACTC
AAGAGGAAGATGGCTGTAGCTGCCGATTTCCAGAAGAAGAAGAAGGAGGATGTGAACTGGCT
AGCCTGAGAGTGAAGTTCAGCAGGAGCGCAGACGCCCCCGCGTACCAGCAGGGCCAGAACCA
GCTCTATAACGAGCTCAATCTAGGACGAAGAGAGGAGTACGATGTTTTGGACAAGAGACGTG
GCCGGGACCCTGAGATGGGGGGAAAGCCGCAGAGAAGGAAGAACCCTCAGGAAGGCCTGTAC
AATGAACTGCAGAAAGATAAGATGGCGGAGGCCTACAGTGAGATTGGGATGAAAGGCGAGCG
CCGGAGGGGCAAGGGGCACGATGGCCTTTACCAGGGTCTCAGTACAGCCACCAAGGACACCT
ACGACGCCCTTCACATGCAGGCCCTGCCCCCTCGCGCTAGC;
(SEQ ID NO: 82)
ATGCTCAGGCTGCTCTTGGCTCTCAACTTATTCCCTTCAATTCAAGTAACAGGAGGGGACGT
ACAACTTGTGGAATCAGGTGGGGGTCTCGTTCAAGCTGGTGGGTCACTGCGGCTTTCTTGCG
CTGCAAGTGGGTTCCCTTATTTTTCCTGCTGTATGAGCTGGCATCGCCAAGCTCCTGGAAAA
GATCGGGAACTGGTCTCCAGTATTTCATCTGACGGGTCAACACATTACGCTGATAGCGTCAG
GGGGAGGTTTACTATTTCTAAAGGAAATGCAAGAAACACACTCTACCTCCAGATGAACAGTC
TGAAGCCCGAAGACACCGCCGTGTATTTCTGTGCAGCTTCCCCTAGACAGCTCGGGCCTTAC
TATGCTATGGACTACTGGGGTCAAGGAACCTCAGTCACCGTCTCCCCGACGCCAGCGCCAAC
TATTGCGAGTCAGCCTCTCAGTCTGCGACCTGAGGCTTGTCGACCAGCAGCCGGAGGCGCAG
TGCACACGAGGGGGCTGGACTTCGCCTGTGATCCTTCTAAGCCCTTTTGGGTGCTGGTGGTG
GTTGGTGGAGTCCTGGCTTGCTATAGCTTGCTAGTAACAGTGGCCTTTATTATTTTCTGGGT
GAGGAAACGGGGCAGAAAGAAACTCCTGTATATATTCAAACAACCATTTATGAGACCAGTAC
AAACTACTCAAGAGGAAGATGGCTGTAGCTGCCGATTTCCAGAAGAAGAAGAAGGAGGATGT
GAACTGGCTAGCCTGAGAGTGAAGTTCAGCAGGAGCGCAGACGCCCCCGCGTACCAGCAGGG
CCAGAACCAGCTCTATAACGAGCTCAATCTAGGACGAAGAGAGGAGTACGATGTTTTGGACA
AGAGACGTGGCCGGGACCCTGAGATGGGGGGAAAGCCGCAGAGAAGGAAGAACCCTCAGGAA
GGCCTGTACAATGAACTGCAGAAAGATAAGATGGCGGAGGCCTACAGTGAGATTGGGATGAA
AGGCGAGCGCCGGAGGGGCAAGGGGCACGATGGCCTTTACCAGGGTCTCAGTACAGCCACCA
AGGACACCTACGACGCCCTTCACATGCAGGCCCTGCCCCCTCGCGCTAGC;
(SEQ ID NO: 83)
ATGCTCAGGCTGCTCTTGGCTCTCAACTTATTCCCTTCAATTCAAGTAACAGGAGGGCAGCT
TCAACTGGTAGAGAGTGGTGGAGGACTTGTTCAGCCTGGAGGGTCCCTTAGACTTAGTTGCG
CTGCCAGTGGGTTCACCCTGGACAACTACGCCATTGGCTGGTTTCGACAGGCCCCAGGGAAA
GAGCGAGAGGGGGTCTCTAGCATCAGTAGTTCCGGTGGTTCCACCAACTATGCTGATAGTGT
GAAGGGGAGGTTTACTGTTTCTCGGGATAATGCAAAACATACCGTGTACCTGCAAATGAATA
GCCTCAAACCAGAAGACACAGCCGTCTATTATTGCGCAGGAGTTGGGGACTACGGTAGAAGG
GCGATGGGTCAAGGCACCTCAGTCACCGTCTCCCCGACGCCAGCGCCAACTATTGCGAGTCA
GCCTCTCAGTCTGCGACCTGAGGCTTGTCGACCAGCAGCCGGAGGCGCAGTGCACACGAGGG
GGCTGGACTTCGCCTGTGATCCTTCTAAGCCCTTTTGGGTGCTGGTGGTGGTTGGTGGAGTC
CTGGCTTGCTATAGCTTGCTAGTAACAGTGGCCTTTATTATTTTCTGGGTGAGGAAACGGGG
CAGAAAGAAACTCCTGTATATATTCAAACAACCATTTATGAGACCAGTACAAACTACTCAAG
AGGAAGATGGCTGTAGCTGCCGATTTCCAGAAGAAGAAGAAGGAGGATGTGAACTGGCTAGC
CTGAGAGTGAAGTTCAGCAGGAGCGCAGACGCCCCCGCGTACCAGCAGGGCCAGAACCAGCT
CTATAACGAGCTCAATCTAGGACGAAGAGAGGAGTACGATGTTTTGGACAAGAGACGTGGCC
GGGACCCTGAGATGGGGGGAAAGCCGCAGAGAAGGAAGAACCCTCAGGAAGGCCTGTACAAT
GAACTGCAGAAAGATAAGATGGCGGAGGCCTACAGTGAGATTGGGATGAAAGGCGAGCGCCG
GAGGGGCAAGGGGCACGATGGCCTTTACCAGGGTCTCAGTACAGCCACCAAGGACACCTACG
ACGCCCTTCACATGCAGGCCCTGCCCCCTCGCGCTAGC;
(SEQ ID NO: 84)
ATGCTCAGGCTGCTCTTGGCTCTCAACTTATTCCCTTCAATTCAAGTAACAGGAGGGCAGCT
TCAACTGGTAGAGAGTGGTGGAGGACTTGTTCAGCCTGGAGGGTCCCTTAGACTTAGTTGCG
CTGCCAGTGGGTTCACCCTGGACTACTACGCCATTGGCTGGTTTCGACAGGCCCCAGGGAAA
GAGCGAGAGGGGGTCTCTTACATCAGTAGTTCCGGTGGTTCCACTAACTACGCTGATAGTGT
GAAGGGGAGGTTTACTATTTCTCGGGATAATGCAAAAAATACCGTGTACCTGCAAATGAATA
GCCTCAAACCAGAAGACACAGGCGTTTATTCTTGCGCAGCAGCTGGGGGCTATGGTAACTCC
TCTCGCTACTGGGGTCAAGGAACCTCAGTCACCGTCTCCCCGACGCCAGCGCCAACTATTGC
GAGTCAGCCTCTCAGTCTGCGACCTGAGGCTTGTCGACCAGCAGCCGGAGGCGCAGTGCACA
CGAGGGGGCTGGACTTCGCCTGTGATCCTTCTAAGCCCTTTTGGGTGCTGGTGGTGGTTGGT
GGAGTCCTGGCTTGCTATAGCTTGCTAGTAACAGTGGCCTTTATTATTTTCTGGGTGAGGAA
ACGGGGCAGAAAGAAACTCCTGTATATATTCAAACAACCATTTATGAGACCAGTACAAACTA
CTCAAGAGGAAGATGGCTGTAGCTGCCGATTTCCAGAAGAAGAAGAAGGAGGATGTGAACTG
GCTAGCCTGAGAGTGAAGTTCAGCAGGAGCGCAGACGCCCCCGCGTACCAGCAGGGCCAGAA
CCAGCTCTATAACGAGCTCAATCTAGGACGAAGAGAGGAGTACGATGTTTTGGACAAGAGAC
GTGGCCGGGACCCTGAGATGGGGGGAAAGCCGCAGAGAAGGAAGAACCCTCAGGAAGGCCTG
TACAATGAACTGCAGAAAGATAAGATGGCGGAGGCCTACAGTGAGATTGGGATGAAAGGCGA
GCGCCGGAGGGGCAAGGGGCACGATGGCCTTTACCAGGGTCTCAGTACAGCCACCAAGGACA
CCTACGACGCCCTTCACATGCAGGCCCTGCCCCCTCGCGCTAGC;
(SEQ ID NO: 85)
ATGCTCAGGCTGCTCTTGGCTCTCAACTTATTCCCTTCAATTCAAGTAACAGGAGGGGAAGT
TCAGCTTGTTGAATCAGGCGGCGGAAGTGTCCAAGCTGGCGAAACCCTTCGGCTCTCCTGCA
CCGCATCCGGCTTCACATTCGATGATTCAGATATGGGCTGGTACAGGCAGGCTCCTGGTGAC
GAATGTGAGCTGGTATCCACAATCTCCAGCGATGGCAGCACAAATTATGCTGACTCTGTTAA
AGGGCGATTTACCATCTCTCAAGATAACGCAAAGAACACCGTGTATCTGCAGATGACCTCAT
TGAAGCCAGAGGATACTGCCATATATTCATGCAGAACAATGGCCTCGAGGATTGGTTGCTGG
GGCCAAGGGACTCTGGTCACTGTCTCTCCGACGCCAGCGCCAACTATTGCGAGTCAGCCTCT
CAGTCTGCGACCTGAGGCTTGTCGACCAGCAGCCGGAGGCGCAGTGCACACGAGGGGGCTGG
ACTTCGCCTGTGATCCTTCTAAGCCCTTTTGGGTGCTGGTGGTGGTTGGTGGAGTCCTGGCT
TGCTATAGCTTGCTAGTAACAGTGGCCTTTATTATTTTCTGGGTGAGGAAACGGGGCAGAAA
GAAACTCCTGTATATATTCAAACAACCATTTATGAGACCAGTACAAACTACTCAAGAGGAAG
ATGGCTGTAGCTGCCGATTTCCAGAAGAAGAAGAAGGAGGATGTGAACTGGCTAGCCTGAGA
GTGAAGTTCAGCAGGAGCGCAGACGCCCCCGCGTACCAGCAGGGCCAGAACCAGCTCTATAA
CGAGCTCAATCTAGGACGAAGAGAGGAGTACGATGTTTTGGACAAGAGACGTGGCCGGGACC
CTGAGATGGGGGGAAAGCCGCAGAGAAGGAAGAACCCTCAGGAAGGCCTGTACAATGAACTG
CAGAAAGATAAGATGGCGGAGGCCTACAGTGAGATTGGGATGAAAGGCGAGCGCCGGAGGGG
CAAGGGGCACGATGGCCTTTACCAGGGTCTCAGTACAGCCACCAAGGACACCTACGACGCCC
TTCACATGCAGGCCCTGCCCCCTCGCGCTAGC;
and
(SEQ ID NO: 86)
ATGCTCAGGCTGCTCTTGGCTCTCAACTTATTCCCTTCAATTCAAGTAACAGGAGGGCAAGT
TCAGCTTGTGGAGTCAGGCGGTGGACTCGTTCAAGCCGGAGGCTCACTGCGGTTGTCTTGTG
TCGCATCTGGTCGAACCTTCTCAAGTTATGCTATGGGGTGGTTCCGCCAGTCTCCTGGTAAA
GAGCGGGAATTTGTCGCAGCAATTTGTTGGTCTGGTGGTAACACTTACTATGCTGATTCAGT
AAAAGGTCGATTCACCATATCAGGGGATAATGCAAAAAATTCTGTCTATCTCCAAATGAACT
CTCTGAAGCCTGAAGATACTGCTGTGTATATCTGTAATGAGAGGACTACGGTACTACCTACG
GGTGCTATGGACTACTGGGGTCAAGGGACCTCAGTCACCGTCTCCTCGCCGACGCCAGCGCC
AACTATTGCGAGTCAGCCTCTCAGTCTGCGACCTGAGGCTTGTCGACCAGCAGCCGGAGGCG
CAGTGCACACGAGGGGGCTGGACTTCGCCTGTGATCCTTCTAAGCCCTTTTGGGTGCTGGTG
GTGGTTGGTGGAGTCCTGGCTTGCTATAGCTTGCTAGTAACAGTGGCCTTTATTATTTTCTG
GGTGAGGAAACGGGGCAGAAAGAAACTCCTGTATATATTCAAACAACCATTTATGAGACCAG
TACAAACTACTCAAGAGGAAGATGGCTGTAGCTGCCGATTTCCAGAAGAAGAAGAAGGAGGA
TGTGAACTGGCTAGCCTGAGAGTGAAGTTCAGCAGGAGCGCAGACGCCCCCGCGTACCAGCA
GGGCCAGAACCAGCTCTATAACGAGCTCAATCTAGGACGAAGAGAGGAGTACGATGTTTTGG
ACAAGAGACGTGGCCGGGACCCTGAGATGGGGGGAAAGCCGCAGAGAAGGAAGAACCCTCAG
GAAGGCCTGTACAATGAACTGCAGAAAGATAAGATGGCGGAGGCCTACAGTGAGATTGGGAT
GAAAGGCGAGCGCCGGAGGGGCAAGGGGCACGATGGCCTTTACCAGGGTCTCAGTACAGCCA
CCAAGGACACCTACGACGCCCTTCACATGCAGGCCCTGCCCCCTCGCGCTAGC.

In any aspect of the disclosure, or embodiments thereof, the mesothelin or fragment thereof is on the surface of a cell. In any aspect of the disclosure, or embodiments thereof, the mesothelin or fragment thereof is endogenously expressed by the cell.

### Definitions

Unless defined otherwise, all technical and scientific terms used herein have the meaning commonly understood by a person skilled in the art to which the various aspects and embodiments of the disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant any small molecule chemical compound, nucleic acid molecule, or polypeptide, or fragments thereof. In various embodiments, the agent is a VHH polypeptide of the disclosure or a polynucleotide encoding the VHH polypeptide.

By “alteration” is meant a change in the structure, sequence, expression levels, or activity of an analyte, clinical indicator, polynucleotide or polypeptide as detected by standard art known methods, such as those described herein. The alteration can be an increase or a reduction.

By “analog” is meant a molecule that is not identical but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

As used herein, the term “antibody” or “antigen-binding domain” refers to an immunoglobulin molecule, a nanobody, or a fragment thereof that specifically binds to, or is immunologically reactive with, a particular antigen. Non-limiting examples of antibodies or antigen-binding domains include VHH antibodies, polyclonal, monoclonal, genetically engineered and otherwise modified forms of antibodies, including but not limited to chimeric antibodies, humanized antibodies, heteroconjugate antibodies (e.g., bi- tri- and quad-specific antibodies, diabodies, triabodies, and tetrabodies), and antigen-binding fragments of antibodies, including e.g., Fab′, F(ab′)₂, Fab, Fv, rlgG, and scFv fragments, as well as engineered antibodies, which include CrossMabs (e.g., CrossMab^{Fabs}, CrossMab^{CH1-CL} and CrossMab^{VH-VL} formats), or fragments thereof. Moreover, unless otherwise indicated, the term “monoclonal antibody” (mAb) is meant to include both intact molecules, as well as antibody fragments (such as, for example, Fab and F(ab′)₂ fragments) that are capable of specifically binding to a target protein. Fab and F(ab′)₂ fragments lack the Fc fragment of an intact antibody, clear more rapidly from the circulation of the animal, and may have less non-specific tissue binding than an intact antibody (see Wahl et al., J. Nucl. Med. 24:316, 1983; incorporated herein by reference).

Antibody structure is well known in the art. Briefly, the variable (V) regions or domains of antibody heavy (H) and light (L) chains contain Complementarity-Determining Regions (CDRs), which bind to specific antigens or immunogens (e.g., protein antigens or immunogens). CDRs are situated within framework (FR) sequences of the V regions of the heavy (V_{H}) and light chains (V_{L}) of an antibody. CDRs are the most variable parts of antibodies and are critical components in the diversity of antigen specificities of antibodies produced by B lymphocytes. In general, three CDRs (CDR1, CDR2 and CDR3) are arranged consecutively in a V domain of an antibody. Because a VHH, such as a camelid VHH, is essentially a single chain antibody polypeptide, it contains three CDRs that bind to an antigen or target protein such as human mesothelin in the context of four framework (FR) regions, as follows: FR1-CDR1-FR2-CDR2-FR3-CDR3-FR4. Because most of the sequence variability associated with immunoglobulins and antigen binding is found in the CDRs, these regions are sometimes referred to as hypervariable regions. Typically, CDR1, CDR2 and CDR3 of VHHs contribute to and/or do not interfere with antigen binding. The CDRs of a number of anti-mesothelin VHHs described herein are shown, for example, in Tables 1, 3A, and 10A to 10M.

By “antigen” is meant an agent to which an antibody or other polypeptide capture molecule specifically binds. In an embodiment, the antigen is a tumor antigen. Exemplary antigens include small molecules, carbohydrates, proteins, and polynucleotides.

A “camelid VHH framework region (FR)” refers to the structural FR portions or components of a camelid VHH antibody or binding molecule, namely, FR1, FR2, FR4 and FR4, that positionally and structurally support the three CDR components, namely, CDR1, CDR2 and CDR3 of a VHH polypeptide, as described above. Similar to the FRs in conventional antibody polypeptides, the respective FR regions (FR1, FR2, FR3 and FR4) of the anti-mesothelin VHH polypeptides described herein are highly similar in sequence not only among different mesothelin binding VHHs but also among camelid VHH polypeptides that bind to other antigens, e.g., unrelated VHH polypeptides. (See, e.g., L. S. Mitchell and L. J. Colwell, 2018, Proteins, 86(7): 697-706 and A. M. Vattekatte et al., March, 2020, Peer J., 6(8):e8408. DOI: 10.7717/peerj.8408). Accordingly, the FR regions FR1, FR2, FR3 and FR4 of different VHHs do not vary significantly in sequence. By way of example, the below FR sequences of the VHH in the above-mentioned publication of Mitchell and Colwell are similar to the FR sequences of other VHHs, including the anti-mesothelin VHH polypeptides described herein.

FR1 (SEQ ID NO: 87):

| Position # | 1 | 2 | 3 | 4 | 5 | 6 | 7 | 8 | 9 | 10 | 11 | 12 | 13 |
|---|---|---|---|---|---|---|---|---|---|---|---|---|---|
| AA | Q | V | Q | L | Q | E | S | G | G | G | L | V | Q |
| | | | | | or | | | | | | or | | |
| | | | | | V | | | | | | S | | |

| Position # | 14 | 15 | 16 | 17 | 18 | 19 | 20 | 21 | 22 | 23 | 24 | 25 |
|---|---|---|---|---|---|---|---|---|---|---|---|---|
| AA | A | G | G | S | L | R | L | S | C | A | A | S |
| | or | | | | | | | | | | | |
| | P | | | | | | | | | | | |

FR2 (SEQ ID NO: 88):

| Position # | 36 | 37 | 38 | 39 | 40 | 41 | 42 | 43 | 44 | 45 | 46 | 47 | 48 | 49 |
|---|---|---|---|---|---|---|---|---|---|---|---|---|---|---|
| AA | W | F, | R | Q | A | P | G | K | E, | R | E | F, | V | A, |
| | | Y, | | | | | | | C, | or | | G, | | S, |
| | | or | | | | | | | or | L | | L, | | or |
| | | V | | | | | | | G | | | or | | T |
| | | | | | | | | | | | | W | | |

FR3 (SEQ ID NO: 89):

| Position # | 60 | 61 | 62 | 63 | 64 | 65 | 66 | 67 | 68 | 69 | 70 | 71 | 72 | 73 | 74 | 75 | 76 | 77 | 78 |
|---|---|---|---|---|---|---|---|---|---|---|---|---|---|---|---|---|---|---|---|
| AA | Y | A, | D | S | V | K | G | R | F | T | I | S | R | D | N | A | K | N | T |
| | | Q, | or | | or | | | | | or | or | | or | | or | | or | | |
| | | T, | E | | A | | | | | A | V | | Q | | K | | A | | |
| | | or | | | | | | | | | | | | | | | | | |
| | | V | | | | | | | | | | | | | | | | | |

| Position # | 79 | 80 | 81 | 82 | 83 | 84 | 85 | 86 | 87 | 88 | 89 | 90 | 91 | 92 | 93 | 94 | 95 | 96 |
|---|---|---|---|---|---|---|---|---|---|---|---|---|---|---|---|---|---|---|
| AA | V, | Y | L | Q | M | N | S | L | K | P | E | D | T | A | V, | Y | Y | C |
| | L, | | | | | or | or | | or | | or | | | or | I, | | | |
| | or | | | | | D | N | | R | | D | | | G | T, | | | |
| | M | | | | | | | | | | | | | | or | | | |
| | | | | | | | | | | | | | | | M | | | |

FR4 (SEQ ID NO: 90):

| Position # | 117 | 118 | 119 | 120 | 121 | 122 | 123 | 124 | 125 | 126 | 127 |
|---|---|---|---|---|---|---|---|---|---|---|---|
| AA | W | G | Q | G | T | Q | V | T | V | S | S |

It will be appreciated that the amino acid position numbers of the VHH FRs shown above are approximate and may vary to some degree in length or amino acid sequence depending on VHH length and on the start and termination amino acid positions of the VHH CDRs. Thus, substantial similarities exist among the structural FRs of camelid VHHs, independent of antigen binding specificity.

A “chimeric antibody” refers to an antibody in which a region of an antibody of one species (e.g., rodent, mouse or rat) is replaced with that from another species (e.g., a human) to achieve a more human-like antibody. Chimeric antibodies may be recombinantly generated by combining the variable light and heavy chain regions obtained from antibody producing cells of one species with the constant light and heavy chain regions from another. In general, chimeric antibodies utilize rodent (or other species, such as rabbit or camelid) variable regions and human constant regions in order to produce an antibody with predominantly human constant domains. The production of chimeric antibodies is well known in the art, and may be achieved by standard means, for example, as described in U.S. Pat. No. 5,624,659, incorporated fully herein by reference.

By “binding to” a molecule is meant having a physicochemical affinity for a molecule (e.g., a protein or protein antigen) or a region of the molecule, e.g., an epitope or antigenic determinant. Binding may be measured by any of the methods practiced in the art, e.g., using an antibody binding assay (e.g., ELISA) or an in vitro translation binding assay.

By “Chimeric Antigen Receptor” or alternatively a “CAR” is meant a polypeptide capable of providing an immune effector cell with specificity for a target cell. In embodiments, the target cell is a cancer cell. In some embodiments, a CAR comprises at least an extracellular antigen binding domain, a transmembrane domain and a cytoplasmic signaling domain comprising a functional signaling domain derived from a stimulatory molecule and/or costimulatory molecule. In embodiments, the stimulatory molecule is the zeta chain associated with the T cell receptor complex. In one embodiment, the cytoplasmic signaling domain further comprises one or more functional signaling domains derived from at least one costimulatory molecule. In one embodiment, the CAR comprises a chimeric fusion protein comprising an extracellular antigen binding domain, a transmembrane domain and an intracellular signaling domain comprising a functional signaling domain derived from a stimulatory molecule. In one embodiment, the CAR comprises a chimeric fusion protein comprising an extracellular antigen binding domain, a transmembrane domain and an intracellular signaling domain comprising a functional signaling domain derived from a costimulatory molecule and a functional signaling domain derived from a stimulatory molecule. In one embodiment, the CAR comprises a chimeric fusion protein comprising an extracellular antigen binding domain, a transmembrane domain and an intracellular signaling domain comprising two functional signaling domains derived from one or more costimulatory molecule(s) and a functional signaling domain derived from a stimulatory molecule. In one embodiment, the CAR comprises a chimeric fusion protein comprising an extracellular antigen binding domain, a transmembrane domain and an intracellular signaling domain comprising at least two functional signaling domains derived from one or more costimulatory molecule(s) and a functional signaling domain derived from a stimulatory molecule. In one embodiment the CAR comprises an optional leader sequence at the amino-terminus (N-ter) of the CAR fusion protein. In one embodiment, the CAR further comprises a leader sequence at the N-terminus of the extracellular antigen binding domain, wherein the leader sequence is optionally cleaved from the antigen binding domain (e.g., a VHH domain) during cellular processing and localization of the CAR to the cellular membrane.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

The term “costimulatory molecule” refers to a cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that contribute to an efficient immune response. Costimulatory molecules include but are not limited to an MHC class I molecule, BTLA and a Toll ligand receptor, as well as OX40, CD27, CD28, CDS, ICAM-1, LFA-1 (CD11a/CD18), ICOS (CD278), and 4-1BB (CD137). Further examples of such costimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11 b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAMI, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include neoplasias, such as tumors and cancers. In various instances, the cancer is a lung cancer, ovarian cancer, mesothelioma, pancreatic adenocarcinoma, lung squamous cell carcinoma, head and neck squamous cell carcinoma, cervical cancer, endometrial cancer, colorectal cancer, gastric cancer, esophageal cancer, or synovial sarcoma.

By “effective amount” is meant the amount of an agent required to ameliorate the symptoms of a disease relative to an untreated patient. The effective amount of active compound(s) used to practice the various aspects and embodiments of the present disclosure for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In some embodiments, an effective amount induces killing of a cancer cell, reduces cancer cell survival, stabilizes tumor growth or cancer cell proliferation, or reduces tumor growth or cancer cell proliferation.

An “epitope tag” refers to a peptide or amino acid sequence (e.g., an epitope) that is fused, linked, or coupled to a protein, such as a recombinant protein produced by recombinant techniques, and that can be specifically bound by an antibody, e.g., an anti-tag monoclonal antibody or binding molecule that is directed to or generated against the tag peptide or amino acid sequence. Epitope tags are typically short peptide sequences (e.g., from about 5-30 amino acids, or sometimes up to 40 amino acids, that are selected because high-affinity antibodies can be reliably produced in many different species. Such anti-epitope tag antibodies are optimally not cross-reactive with other human peptides or polypeptides and typically do not generate an antibody response, e.g., an anti-tag antibody response, when administered or delivered to a subject. An epitope tag sequence that is fused to a protein provides for the detection and/or purification of the protein using an antibody, e.g., a monoclonal antibody, that specifically binds to the epitope tag. In an embodiment, the protein to which an epitope tag is fused, linked, or coupled is an antibody or VHH protein, e.g., a recombinantly produced antibody or VHH protein. In an embodiment, the VHH is an anti-mesothelin VHH antibody. In an embodiment, the protein, may include one or more epitope tags. In an embodiment, an epitope tag is coupled to the amino (NH) terminus of the protein, e.g., a VHH antibody as described herein. In an embodiment, an epitope tag is coupled to the carboxy (COOH) terminus of the protein, e.g., a VHH antibody as described herein. In an embodiment, an epitope tag is coupled to the NH and the COOH termini of the protein, e.g., a VHH antibody as described herein. An epitope tag sequence such as those described herein may be bound by anti-epitope tag antibodies, forming complexes which may facilitate clearance of the protein containing the tags from the body or system. (See, also, B. Brizzard and R. Chubet, 2001, Curr Protoc Neurosci., Chapter 5, Unit 5.8; DOI: 10.1002/0471142301.ns0508s00; R. Hernan et al., 2000, Biotechniques, 28(4):789-793; C. E. Fritze et al., 2000, Meths Enzymol., 327:3-16; doi: 10.1016/s0076-6879(00)27263-7; A. Einhauer et al., 2001, J Biochem Biophys Methods, 49(1-3):455-65, doi: 10.1016/s0165-022x(01)00213-5)).

Other molecules may serve as protein, amino acid sequence, or polynucleotide tags that are fused, linked, or coupled to a protein, such as a recombinant protein produced by recombinant techniques, e.g., an anti-mesothelin VHH antibody described herein. In an embodiment, the tag can be specifically bound by an antibody, e.g., an anti-tag monoclonal antibody or binding molecule that is directed to or generated against the tag peptide or amino acid sequence. Examples of tags include, without limitation, FLAG tags (peptide sequence DYKDDDDK (SEQ ID NO: 91) recognized by an anti-FLAG antibody), polyHistidine (His) tags (5-10 histidine residues (SEQ ID NO: 92) (HHHHHH (SEQ ID NO: 93)) bound by a nickel or cobalt chelate), E-tag, a peptide comprising amino acid sequence GAPVPYPDPLEPR (SEQ ID NO: 94) recognized by an antibody; an immunoglobulin Fc region or portion thereof, e.g., having effector or modulator function (Fc tag). In particular, Fc tags comprise a domain (effector domain) of an immunoglobulin molecule, e.g., IgG, which can be genetically linked to a peptide or protein. Ec fusion proteins (also known as Fe chimeric fusion proteins, Fc-Igs, Ig-based chimeric fusion proteins, and Fc-tag proteins) are composed of an Ig Fc domain that is fused, linked, or coupled (e.g., by recombinant techniques) to a peptide or protein, such as an anti-mesothelin VHH antibody described herein. The Fc domain portion of the fusion protein confers an advantageous characteristic to the protein, particularly in vivo, by greatly prolonging the half-life of the protein in plasma following administration to a subject. In an embodiment, an anti-mesothelin VHH antibody fused to an Fc region or Fc tag provides improved therapeutic efficacy as a biotherapeutic agent or drug. Fe fusion proteins also have uses in in vitro methods, including, e.g., immunohistochemistry (IHC), flow cytometry (FC), protein binding assays and use as microarray baits. In these applications, the Fc domain serves as a support to which proteins can be attached while retaining their native biological activity. In addition, the Fc domain can improve the in vivo and in vitro solubility and stability of the protein or peptide molecule to which it is coupled, fused, linked, or attached.

By “fragment” is meant a portion of a polypeptide or nucleic acid molecule. In embodiments, portion contains, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, 100, 110, 120, 130, 140, 150, 200, 300, 400, 500, 600, 700, 800, 900, or 1000 nucleotides or amino acids.

A “framework (FR) region” or “FR region” includes amino acid residues that are adjacent to the CDRs in V_{H}, and V_{L} regions, and in VHHs. For example, FR region residues may be present in VHHs as described herein, camelid antibodies (VHHs), human antibodies, rodent-derived antibodies (e.g., murine and rat antibodies), humanized antibodies, primatized antibodies, chimeric antibodies, antibody fragments (e.g., Fab fragments), VHHs, single-chain antibody fragments (e.g., scFv fragments), antibody domains, and bispecific antibodies, among others.

The term “humanized” antibodies refers to forms of non-human (e.g., murine) antibodies, camelid-derived single domain antibody (sdAb) binding molecules, which are comprised of the heavy chain variable (V_{H}) region of heavy-chain-only antibodies (Abs) or VHHs. Humanized antibodies include chimeric immunoglobulins, immunoglobulin chains or fragments thereof (such as Fv, Fab, Fab′, F(ab′)₂ or other target-binding subdomains of antibodies) which contain minimal sequences derived from non-human immunoglobulin. In general, a humanized antibody or VHH may comprise substantially all of at least one variable domain (or two variable domains in the case of non-VHH antibodies), in which all or substantially all of the CDR regions correspond to those of a non-human immunoglobulin. All or substantially all of the FR regions of a humanized antibody may also be derived from a human immunoglobulin sequence. In the case of non-VHH antibodies, a VHH or a humanized antibody can also comprise at least a portion of an immunoglobulin constant region (Fc), which may be that of a human immunoglobulin consensus sequence. Techniques and protocols for humanizing antibodies (as well as VHHs) are known and practiced in the art, as described, for examples, in Riechmann et al., Nature, 332:323-7, 1988; Kasmiri et al., Methods, 36(1):25-34, 2005; U.S. Pat. Nos. 5,530,101; 5,585,089; 5,693,761; 5,693,762; and U.S. Pat. No. 6,180,370 to Queen et al; EP239400; WO 1991/09967; U.S. Pat. No. 5,225,539; EP592106; and EP519596, the contents of which are incorporated herein by reference. Humanized antibodies or VHHs are molecularly engineered to contain even more human-like immunoglobulin domains, and incorporate only the CDRs of the VHH or animal-derived monoclonal antibody by carefully examining the sequence of the hyper-variable loops of the V regions of the monoclonal antibody or VHH, and fitting them to the structure of the human antibody chains. This process is routinely and commonly carried out by one having skill in the art. See, e.g., U.S. Pat. No. 6,187,287, the contents of which are incorporated by reference herein.

By “increase” is meant to alter positively by at least 5% relative to a reference. An increase may be by 5%, 10%, 25%, 30%, 50%, 75%, or even by 100%.

An “intracellular signaling domain,” refers to portion of a molecule that transduces a signal from the surface of the cell to the interior of the cell. The intracellular signaling domain generates a signal that promotes an immune effector function of a CAR containing cell, e.g., a CART cell. Examples of immune effector function, e.g., in a CART cell, include cytolytic activity and helper activity, including the secretion of cytokines.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid molecule that is free of the genes which, in the naturally-occurring genome of the organism from which the nucleic acid molecule of the disclosure is derived, flank the gene. The term therefore includes, for example, a recombinant DNA that is incorporated into a vector; into an autonomously replicating plasmid (e.g., a pLX311 plasmid or a pLX307 plasmid) or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the disclosure that has been separated from components that naturally accompany it. Typically, the polypeptide is isolated when it is at least 60%, by weight, free from the proteins and naturally-occurring organic molecules with which it is naturally associated. In embodiments, the preparation is at least 75%, at least 90%, and or at least 99%, by weight, a polypeptide of the disclosure. An isolated polypeptide of the disclosure may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any clinical indicator, analyte, protein or polynucleotide having an alteration in expression level or activity that is associated with a condition, disease, or disorder. A non-limiting example of a marker is a mesothelin polypeptide.

By “mesothelin polypeptide (MSLN)” is meant a protein with at least about 85% amino acid sequence identity to Genbank Accession No. AAH03512.1, or a fragment thereof that is immunogenic in a subject. An exemplary mesothelin amino acid sequence from Homo sapiens is provided below (GenBank: AAH03512.1):

(SEQ ID NO: 95)
>AAH03512.1 Mesothelin [Homo sapiens]
MALPTARPLLGSCGTPALGSLLFLLFSLGWVQPSRTLAGETGQAAPLDG
VLANPPNISSLSPRQLLGFPCAEVSGLSTERVRELAVALAQKNVKLSTE
QLRCLAHRLSEPPEDLDALPLDLLLFLNPDAFSGPQACTRFFSRITKAN
VDLLPRGAPERQRLLPAALACWGVRGSLLSEADVRALGGLACDLPGRFV
AESAEVLLPRLVSCPGPLDQDQQEAARAALQGGGPPYGPPSTWSVSTMD
ALRGLLPVLGQPIIRSIPQGIVAAWRQRSSRDPSWRQPERTILRPRFRR
EVEKTACPSGKKAREIDESLIFYKKWELEACVDAALLATQMDRVNAIPF
TYEQLDVLKHKLDELYPQGYPESVIQHLGYLFLKMSPEDIRKWNVTSLE
TLKALLEVNKGHEMSPQVATLIDRFVKGRGQLDKDTLDTLTAFYPGYLC
SLSPEELSSVPPSSIWAVRPQDLDTCDPRQLDVLYPKARLAFQNMNGSE
YFVKIQSFLGGAPTEDLKALSQQNVSMDLATFMKLRTDAVLPLTVAEVQ
KLLGPHVEGLKAEERHRPVRDWILRQRQDDLDTLGLGLQGGIPNGYLVL
DLSVQEALSGTPCLLGPGPVLTVLALLLASTLA.

By “mesothelin polynucleotide” is meant a nucleic acid molecule encoding a mesothelin polypeptide, as well as the introns, exons, 3′ untranslated regions, 5′ untranslated regions, and regulatory sequences associated with its expression, or fragments thereof. In embodiments, a mesothelin polynucleotide is the genomic sequence, cDNA, mRNA, or gene associated with and/or required for mesothelin expression. An exemplary mesothelin nucleotide sequence from Homo sapiens is provided below (GenBank: BC003512.1:418-2283):

>BC003512.1:418-2283 Homo sapiens mesothelin, mRNA (cDNA clone
MGC: 10686 IMAGE: 3611296), complete cds
(SEQ ID NO: 96)
ATGGCCTTGCCAACGGCTCGACCCCTGTTGGGGTCCTGTGGGACCCCCGCCCTCGGCAGCCT
CCTGTTCCTGCTCTTCAGCCTCGGATGGGTGCAGCCCTCGAGGACCCTGGCTGGAGAGACAG
GGCAGGCTGCACCCCTGGACGGAGTCCTGGCCAACCCACCTAACATTTCCAGCCTCTCCCCT
CGCCAACTCCTTGGCTTCCCGTGTGCGGAGGTGTCCGGCCTGAGCACGGAGCGTGTCCGGGA
GCTGGCTGTGGCCTTGGCACAGAAGAATGTCAAGCTCTCAACAGAGCAGCTGCGCTGTCTGG
CTCACCGGCTCTCTGAGCCCCCCGAGGACCTGGACGCCCTCCCATTGGACCTGCTGCTATTC
CTCAACCCAGATGCGTTCTCGGGGCCCCAGGCCTGCACCCGTTTCTTCTCCCGCATCACGAA
GGCCAATGTGGACCTGCTCCCGAGGGGGGCTCCCGAGCGACAGCGGCTGCTGCCTGCGGCTC
TGGCCTGCTGGGGTGTGCGGGGGTCTCTGCTGAGCGAGGCTGATGTGCGGGCTCTGGGAGGC
CTGGCTTGCGACCTGCCTGGGCGCTTTGTGGCCGAGTCGGCCGAAGTGCTGCTACCCCGGCT
GGTGAGCTGCCCGGGACCCCTGGACCAGGACCAGCAGGAGGCAGCCAGGGCGGCTCTGCAGG
GCGGGGGACCCCCCTACGGCCCCCCGTCGACATGGTCTGTCTCCACGATGGACGCTCTGCGG
GGCCTGCTGCCCGTGCTGGGCCAGCCCATCATCCGCAGCATCCCGCAGGGCATCGTGGCCGC
GTGGCGGCAACGCTCCTCTCGGGACCCATCCTGGCGGCAGCCTGAACGGACCATCCTCCGGC
CGCGGTTCCGGCGGGAAGTGGAGAAGACAGCCTGTCCTTCAGGCAAGAAGGCCCGCGAGATA
GACGAGAGCCTCATCTTCTACAAGAAGTGGGAGCTGGAAGCCTGCGTGGATGCGGCCCTGCT
GGCCACCCAGATGGACCGCGTGAACGCCATCCCCTTCACCTACGAGCAGCTGGACGTCCTAA
AGCATAAACTGGATGAGCTCTACCCACAAGGTTACCCCGAGTCTGTGATCCAGCACCTGGGC
TACCTCTTCCTCAAGATGAGCCCTGAGGACATTCGCAAGTGGAATGTGACGTCCCTGGAGAC
CCTGAAGGCTTTGCTTGAAGTCAACAAAGGGCACGAAATGAGTCCTCAGGTGGCCACCCTGA
TCGACCGCTTTGTGAAGGGAAGGGGCCAGCTAGACAAAGACACCCTAGACACCCTGACCGCC
TTCTACCCTGGGTACCTGTGCTCCCTCAGCCCCGAGGAGCTGAGCTCCGTGCCCCCCAGCAG
CATCTGGGCGGTCAGGCCCCAGGACCTGGACACGTGTGACCCAAGGCAGCTGGACGTCCTCT
ATCCCAAGGCCCGCCTTGCTTTCCAGAACATGAACGGGTCCGAATACTTCGTGAAGATCCAG
TCCTTCCTGGGTGGGGCCCCCACGGAGGATTTGAAGGCGCTCAGTCAGCAGAATGTGAGCAT
GGACTTGGCCACGTTCATGAAGCTGCGGACGGATGCGGTGCTGCCGTTGACTGTGGCTGAGG
TGCAGAAACTTCTGGGACCCCACGTGGAGGGCCTGAAGGCGGAGGAGCGGCACCGCCCGGTG
CGGGACTGGATCCTACGGCAGCGGCAGGACGACCTGGACACGCTGGGGCTGGGGCTACAGGG
CGGCATCCCCAACGGCTACCTGGTCCTAGACCTCAGCGTGCAAGAGGCCCTCTCGGGGACGC
CCTGCCTCCTAGGACCTGGACCTGTTCTCACCGTCCTGGCACTGCTCCTAGCCTCCACCCTG
GCCTGA.

A “nanobody” as used herein refers to a single-domain antibody or VHH. For example, a nanobody refers to an antibody fragment or portion which contains a single monomeric variable domain (V_{H}) naturally occurring in the Camelidae family or synthetically derived from the heavy chain of an antibody. Such single-domain binding molecules combine high antigen affinity in the absence of complement-dependent or cell-mediated cytotoxicity due to the lack of a constant (Fc) region in these molecules.

By “neoplasia” is meant a disease or disorder characterized by excess proliferation or reduced apoptosis. In embodiments, a neoplasia is a cancer or tumor. Illustrative neoplasms include breast cancer, esophageal cancer, head-and-neck cancer, pancreatic cancer, skin cancer, colorectal cancer, hepatocellular cancer, bladder cancer, bile duct cancer, luminal and non-luminal bladder cancer, basal bladder cancer, muscle-invasive bladder cancer, and non-muscle-invasive bladder cancer, pancreatic cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, liver cancer, cervical cancer, uterine cancer, testicular cancer, lung carcinoma, small cell lung carcinoma, bladder carcinoma, epithelial carcinoma, glioma, glioblastoma multiforme, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). In embodiments, the neoplasia may be colon adenocarcinoma (COAD), stomach adenocarcinoma (STAD), stomach cancer, and uterine corpus endometrial carcinoma (UCEC). In embodiments, the neoplasia may be a liquid tumor such as, for example, leukemia or lymphoma. In embodiments, the cancer is a colon, kidney, lung, pancreatic, renal (e.g., renal cell carcinoma or clear renal cell carcinoma), or skin cancer (e.g., a melanoma). In embodiments, the neoplasia is a mesothelioma, pancreatic adenocarcinoma, lung squamous cell carcinoma, head and neck squamous cell carcinoma, cervical cancer, endometrial cancer, colorectal cancer, gastric cancer, esophageal cancer, or synovial sarcoma.

As used herein, “obtaining” as in “obtaining an agent” includes synthesizing, purchasing, or otherwise acquiring the agent.

By “operably linked” is meant the connection between regulatory elements and one or more polynucleotides (genes) or a coding region. That is, gene expression is typically placed under the control of certain regulatory elements, including constitutive or inducible promoters, tissue-specific regulatory elements, and enhancers. A polynucleotide (gene or genes) or coding region is said to be “operably linked to” or “operatively linked to” or “operably associated with” the regulatory elements, meaning that the polynucleotide (gene or genes) or coding region is controlled or influenced by the regulatory elements. The one or more polynucleotides may be separated by spacers or linkers.

“Percentage of sequence identity” means the value determined by comparing two optimally aligned sequences over a comparison window, wherein the portion of the polynucleotide sequence in the comparison window may comprise additions, substitutions, or deletions (i.e., gaps) as compared to the reference sequence (which does not comprise additions, substitutions, or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

By “polypeptide” or “amino acid sequence” is meant any chain of amino acids, regardless of length or post-translational modification. In various embodiments, the post-translational modification is glycosylation or phosphorylation. In various embodiments, conservative amino acid substitutions may be made to a polypeptide to provide functionally equivalent variants, or homologs of the polypeptide. In some cases, the various aspects of the disclosure embrace sequence alterations that result in conservative amino acid substitutions. In some embodiments, a “conservative amino acid substitution” refers to an amino acid substitution that does not alter the relative charge or size characteristics of the protein in which the conservative amino acid substitution is made. Variants can be prepared according to methods for altering polypeptide sequence known to one of ordinary skill in the art such as are found in references that compile such methods, e.g., Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Non-limiting examples of conservative substitutions of amino acids include substitutions made among amino acids within the following groups: (a) M, I, L, V; (b) F, Y, W; (c) K, R, H; (d) A, G; (e) S, T; (f) Q, N; and (g) E, D. In various embodiments, conservative amino acid substitutions can be made to the amino acid sequence of the proteins and polypeptides disclosed herein.

By “reduce” is meant to alter negatively by at least 5% relative to a reference. A reduction may be by 5%, 10%, 25%, 30%, 50%, 75%, or even by 100%.

By “reference” is meant a standard or control condition. In embodiments, a reference cell is a cell that does not express one of the VHH polypeptides presented herein. In some embodiments, a reference is a non-VHH anti-mesothelin antibody that does not bind mesothelin.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 10 amino acids, at least about 20 amino acids, at least about 25 amino acids, at least about 35 amino acids, at least about 50 amino acids, or at least about 100 amino acids. For nucleic acids, the length of the reference nucleic acid sequence will generally be at least about 50 nucleotides, at least about 60 nucleotides, at least about 75 nucleotides, at least about 100 nucleotides, or at least about 300 nucleotides, or any integer thereabout or therebetween. In embodiments, a reference sequence is a VHH antibody or a fragment thereof that does not bind a target antigen. In some cases, a reference sequence is a VHH antibody or a fragment thereof that binds a target antigen.

By “specifically binds” is meant recognizes and binds a polypeptide of the disclosure, but which does not substantially recognize and bind other molecules in a sample. In embodiments, a capture molecule is a VHH domain or a fragment thereof. A VHH domain or fragment thereof that specifically binds to an antigen will bind to the antigen with a K_{D} of less than 100 nM. For example, a VHH domain or fragment thereof that specifically binds to an antigen will bind to the antigen with a K_{D} of up to 100 nM (e.g., between 1 pM and 100 nM). A VHH domain or fragment thereof that does not exhibit specific binding to a particular antigen or epitope thereof will exhibit a K_{D} of greater than 100 nM (e.g., greater than 500 nm, 1 uM, 100 uM, 500 uM, or 1 mM) for that particular antigen or epitope thereof. A variety of immunoassay formats may be used to select a VHH domain or fragment thereof that specifically immunoreactive with a particular protein or carbohydrate. For example, solid-phase ELISA immunoassays are routinely used to select VHH domains or fragments thereof specifically immunoreactive with a protein or carbohydrate. See, Harlow & Lane, Antibodies, A Laboratory Manual, Cold Spring Harbor Press, New York (1988) and Harlow & Lane, Using Antibodies, A Laboratory Manual, Cold Spring Harbor Press, New York (1999), for a description of immunoassay formats and conditions that can be used to determine specific immunoreactivity.

Nucleic acid molecules useful in the methods of the disclosure include any nucleic acid molecule that encodes a polypeptide of the disclosure or a fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the disclosure include any nucleic acid molecule that encodes a polypeptide of the disclosure or a fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, about less than about 500 mM NaCl and 50 mM trisodium citrate, or about less than about 250 mM NaCl and 25 mM trisodium citrate. Low stringency hybridization can be obtained in the absence of organic solvent, e.g., formamide, while high stringency hybridization can be obtained in the presence of at least about 35% formamide, or at least about 50% formamide. Stringent temperature conditions will ordinarily include temperatures of at least about 30° C., of at least about 37° C., or of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In one embodiment, hybridization will occur at 30° C. in 750 mM NaCl, 75 mM trisodium citrate, and 1% SDS. In another embodiment, hybridization will occur at 37° C. in 500 mM NaCl, 50 mM trisodium citrate, 1% SDS, 35% formamide, and 100 μg/ml denatured salmon sperm DNA (ssDNA). In another embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will be less than about 30 mM NaCl and 3 mM trisodium citrate, or less than about 15 mM NaCl and 1.5 mM trisodium citrate. Stringent temperature conditions for the wash steps will ordinarily include a temperature of at least about 25° C., of at least about 42° C., or of at least about 68° C. In one embodiment, wash steps will occur at 25° C. in 30 mM NaCl, 3 mM trisodium citrate, and 0.1% SDS. In another embodiment, wash steps will occur at 42 C in 15 mM NaCl, 1.5 mM trisodium citrate, and 0.1% SDS. In another embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In embodiments, such a sequence is at least 60%, at least 80% or 85%, or at least about 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant an animal. The animal can be a mammal. The mammal can be a human or non-human mammal, such as a bovine, equine, canine, ovine, rodent, or feline.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

“Transduction” refers to a process by which a polynucleotide is introduced or transferred into a cell. In an embodiment, the DNA or polynucleotide transduced into a cell is stably expressed in the cell. In some cases, the virus or virus vector is said to infect a cell.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the term “vector” refers to a polynucleotide suitable for delivery of a gene sequence to a cell, or to a virus particle. Non-limiting examples of vectors include plasmids (e.g., a pLX311 plasmid or a pLX307 plasmid) and cosmids. A “vector” further refers to a nucleic acid (polynucleotide) molecule into which foreign nucleic acid can be inserted without disrupting the ability of the vector to be expressed in, replicate in, and/or integrate into a host cell. A variety of vectors have been developed for the delivery of polynucleotides encoding exogenous proteins into a prokaryotic or eukaryotic cell. A vector may contain a polynucleotide sequence that includes gene of interest (e.g., a heterologous gene, such as a therapeutic gene, or a reporter gene) as well as, for example, additional sequence elements capable of regulating transcription, translation, and/or the integration of these polynucleotide sequences into the genome of a cell. A vector may contain regulatory sequences, such as a promoter, e.g., a subgenomic promoter, region, and an enhancer region, which direct gene transcription. A vector may contain polynucleotide sequences (enhancer sequences) that enhance the rate of translation of these genes or improve the stability or nuclear export of the mRNA that results from gene transcription. These sequence elements may include, e.g., 5′ and 3′ untranslated regions, an internal ribosomal entry site (IRES), and/or a polyadenylation signal site to direct efficient transcription of a gene carried on the expression vector. Vectors, such as the viral particles described herein, may also be referred to as expression vectors.

As used herein, the term “vehicle” refers to a solvent, diluent, or carrier component of a pharmaceutical composition.

By “viral particle” is meant an agent capable of infecting a cell and that exists as an independent particle containing a core viral genome or polynucleotide, a capsid, which surrounds the genetic material and protects it, and an envelope of lipids surrounding the capsid. A viral particle may refer to the form of a virus before it infects a cell and becomes intracellular, or to the form of the virus that infects a cell.

By “VHH domain” is meant an antigen binding domain of a heavy chain only antibody or an antigen binding fragment thereof.

A “VHH binding molecule” or “VHH antibody,” or simply “VHH,” as referred to herein is, in general, a single domain immunoglobulin molecule (antibody). A VHH (or VHH antibody) corresponds to the heavy chain of a VHH antibody having a single variable domain (or single variable region), e.g., a camelid-derived single variable H (V_{H}) domain antibody. A VHH typically has a molecular weight (MW) of about 12-15 kDa. VHH antibodies lack light chains. These heavy-chain antibody molecules contain a single variable domain (VHH) and, typically, two constant domains (CH₂ and CH₃). See, e.g., Methods in Molecular Biology, “Single Domain Antibodies—Methods and Protocols,” Eds. D. Saerens and S. Muyldermans, Humana Press (Springer), 2012. A cloned (recombinantly produced) and isolated VHH domain is a stable polypeptide harboring the antigen-binding capacity of the original heavy-chain antibody. See, e.g., U.S. Pat. Nos. 5,840,526 and 6,015,695, each of which is incorporated by reference herein in its entirety.

VHHs are efficiently expressed in E. coli, coupled to detection markers, such as a fluorescent marker, or conjugated with enzymes. The small size of VHHs permits their binding to epitopes (antigenic determinants in antigen proteins), e.g., “hidden epitopes” that are not accessible to whole antibodies of much larger size. As a therapeutic, a VHH is capable of efficient penetration and rapid clearance. Its single domain nature allows a VHH to be expressed in a cell without a requirement for supramolecular assembly, as is needed for whole antibodies which are typically tetrameric (two heavy chains and two light chains, having a MW of about 150 kDa). VHHs are also exhibit stability over time and have a longer half-life versus non-VHH antibody molecules, which comprise disulfide bonds that are susceptible to chemical reduction or enzymatic cleavage. Similar to immunoglobulins, VHHs may be modified post-translationally, e.g., to add chemical linkers, detectable moieties, such as fluorescent dyes, enzymes, substrates, chemiluminescent moieties, etc., or specific binding moieties, such as streptavidin, avidin, or biotin, etc., for use in the compositions and methods described herein.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 a multiple sequence alignment of anti-mesothelin VHH antibodies of the disclosure. Figure discloses SEQ ID NOS 61, 59, 58, 64, 60, 62, 57, 63, 55, 65, 56, and 66, respectively, in order of appearance.

FIG. 2 provides a schematic diagram relating to whole-cell vaccination using a DC2.4 cell line. Antigens of interest can be introduced to the DC2.4 cells through lentiviral transduction using a lentiviral vector containing a polynucleotide encoding an antigen of interest. Antigens expressed in the DC2.4 cells are presented by MHC class I and/or class II molecules on the surface of the cells. FIG. 2 provides a schematic diagram showing a strategy for immunizing animals using DC2.4 cells expressing a target antigen. The DC2.4 cells are stimulated with CpG oligodeoxynucleotides for about 4 hours prior to vaccinating an animal (e.g., a mouse) with about 5 million cells by intraperitoneal injection (IP) and subcutaneous injection. After about two weeks, the animals are administered a boost vaccination. After about two additional weeks, cells (e.g., antibody-producing B cells) are harvested from the animals, or the animals are administered further boost vaccinations.

FIG. 3 provides a schematic diagram showing a protocol for preparation and characterization of chimeric antigen receptors containing anti-mesothelin VHH domains that were produced by nanomice vaccinated using a live vaccine surface-presenting mesothelin.

FIG. 4 presents flow cytometry plots demonstrating that Jurkat cells surface-expressing chimeric antigen receptors (CARs) containing anti-mesothelin VHH domains provided herein as antigen binding domains were activated when cultured in the presence of target cells surface-expressing mesothelin (MSLN).

FIG. 5 provides plots showing activation of Jurkat cells surface-expressing chimeric antigen receptors (CARs) containing anti-mesothelin VHH domains provided herein as antigen binding domains were activated when cultured in the presence of target cells surface-expressing mesothelin (MSLN). In FIG. 5, “CAR-Ts only target_line” indicates Jurkat cells cultured in the absence of any target cells, “MSLN-target_line” indicates Jurkat cells co-cultured with cells that did not surface-present mesothelin, and “MSLN+target_line” indicates Jurkat cells co-cultured with target cells that surface-presented mesothelin. In FIG. 5 the top portion of each plot indicates the identity of the “MSLN+” target cells, namely either A549 cells, DC2.4 cells surface-expressing mesothelin, or K562 cells.

FIGS. 6A and 6B provide maps for a plasmid used in the Examples to express chimeric antigen receptors containing a VHH domain of the disclosure. FIG. 6A provides a map for the full plasmid and FIG. 6B provides a map for the portion of the plasmid encoding a chimeric antigen receptor linked to enhanced green fluorescent protein (eGFP) by way of a P2A self-cleaving peptide.

FIGS. 7A and 7B provide maps for a plasmid used in the Examples to detect activation of chimeric antigen receptor (CAR) T cells. FIG. 7A provides a map for the full plasmid and FIG. 7B provides a map of the portion of the plasmid corresponding to a reporter cassette.

FIGS. 8A to 8G provide flow cytometry contour plots, plots, and a flow cytometry histogram validating the ability of T cells expressing chimeric antigen receptors (CARs) containing anti-mesothelin (anti-MSLN) VHH domains of the disclosure (anti-MSLN CAR-T cells) as antigen binding domains to kill target tumor cells in mice. FIGS. 8A and 8B provide flow cytometry contour plots and a plot demonstrating expansion of anti-MSLN CAR-T cells expressing CARs containing the indicated antigen binding domains (i.e., Ab_113415, Ab_119702, Ab_119704, Ab_119706, and SS1). Expression of enhanced green fluorescent protein (eGFP) directly correlated with expression of the CARs in the cells. FIG. 8C provides a flow cytometry histogram demonstrating that >99% of K562_pLX311-MSLN target cancer cells (target K562 cells) administered to the mice surface expressed mesothelin. FIG. 8D provides a plot showing results from an in vitro cytotoxicity assay demonstrating the ability of the CAR-T cells to kill the target K562 cells. In FIG. 8D, “negative” indicates K562 cells that did not surface express mesothelin, and “positive” indicates K562 cells that surface expressed mesothelin. FIGS. 8E and 8F provide plots demonstrating the ability of the CAR-T cells to kill target K562 cells and control tumor growth in mice. The mice were subcutaneously administered 1.5e6 of target K562 cells prior to being administered 5e6 of the CAR-T cells via tail vein injection three days following administration of the target K562 cells. FIG. 8G provides a plot showing improved survival of mice administered the CAR-T cells. In FIGS. 8A to 8G the term “unmodified” refers to T cells that did not express a chimeric antigen receptor, the term “tumor only” refers to mice that were only administered the target K562 cells and no CAR-T cells, and the term “6-Well GREX” refers to G-Rex®6 Well Plates. The CARs were cloned and expressed in the cells as described in Example 1. The anti-MSLN antigen binding domain SS1 was used as a positive control. In FIG. 8G, the y-axis represents percent surviving mice and the x-axis represents time in days post tumor challenge.

FIGS. 9A and 9B provide plots and flow cytometry contour plots characterizing the ability of VHH antibodies of the disclosure to bind mesothelin (MSLN). FIG. 9A provides a plot demonstrating the ability of the indicated single domain antibody binding molecules to bind K562_pLX311-MSLN cells surface-expressing mesothelin. In FIG. 9A the term “Secondary Only” indicates cells contacted only with a secondary antibody and not with a single domain antibody binding molecule, “MSLN_pos” indicates cells surface expressing mesothelin, “MSLN_neg” indicates cells that did not surface express mesothelin, “w/MSLN” indicates cells contacted with media containing soluble mesothelin, and “w/o MSLN” indicates cells contacted with media that did not contain soluble mesothelin. FIG. 9B provides flow cytometry contour plots showing the ability of the CAR-T cells of FIGS. 8A to 8G to bind soluble mesothelin.

FIG. 10 provides overlaid flow cytometry histograms demonstrating the ability of VHH antibodies of the disclosure to bind mesothelin (MSLN) endogenously expressed on the surface of the AsPC-1 human pancreatic cell line. In FIG. 10, “Control” indicates cells that were not contacted with any VHH antibody of the disclosure, and “Commercial” indicates cells that were contacted with the commercially available antibody FAB32652A.

FIG. 11 provides a plot showing clustering of VHH antibody sequences of the disclosure. The starred data points indicate sequences corresponding to the indicated VHH antibody.

FIG. 12 provides a plot showing clustering of VHH antibody sequences of the disclosure. The starred data points indicate sequences corresponding to the indicated VHH antibody.

### DETAILED DESCRIPTION

Described herein are single domain antibody (sdAb) binding molecules, which are comprised of the heavy chain variable (V_{H}) region of heavy-chain-only antibodies (Abs), that specifically bind to a mesothelin polypeptide, in particular, a human mesothelin polypeptide expressed by a neoplastic cell. In some embodiments, the present disclosure provided chimeric antigen receptors (CAR) comprising a VHH polypeptide of the disclosure as an antigen binding domain, and cells (e.g., CAR-T cells) expressing the same.

Single-domain antibodies (camelid single-domain antibodies or nanobodies) are called VHHs as they derive from the V_{H} region of a class of heavy-chain-only antibodies. The anti-mesothelin VHHs were produced from immunized camelid animals (alpacas) and were selected for their ability to specifically bind to mesothelin. The anti-mesothelin VHHs as described herein are mesothelin-binding polypeptides comprising hypervariable variable regions (CDRs) within framework (FR) regions. In general, the FRs of the anti-mesothelin VHHs are typically highly similar in amino acid sequence or differ by conservative amino acid substitutions at certain positions of the FR sequences, among different anti-mesothelin VHHs or families of anti-mesothelin VHHs.

The anti-mesothelin VHHs as described herein provide advantageous properties, particularly for therapeutic use. By way of nonlimiting example, these sdAb molecules are small proteins (e.g., about 14 Kda), thus facilitating the cloning of their encoding polynucleotides. The anti-mesothelin VHHs can be functionally expressed at high levels, are stable to extreme pH and high temperatures over time, and function well in multimeric forms, e.g., dimers and other multimers, to provide improved binding and neutralization properties and therapeutic efficacy.

Chimeric antigen receptor (CAR) T cells expressing chimeric antigen receptors containing VHHs of the disclosure may be employed as therapeutic agents for the treatment and prevention of mesothelin-mediated and associated disorders, conditions, or diseases as described herein. In embodiments, a VHH of the disclosure is conjugated to a cytotoxic agent capable of killing a mesothelin-expressing neoplastic cell. It will be understood that the terms “anti-mesothelin VHH antibody,” “anti-mesothelin VHH polypeptide,” “anti-mesothelin VHH antibody polypeptide,” “anti-mesothelin VHH,” and “mesothelin VHH” are used interchangeably herein.

### Mesothelin

Mesothelin, also known as MSLN, is a protein that in humans is encoded by the MSLN gene. Mesothelin is a 40 kDa protein that is expressed in mesothelial cells. The protein was first identified by its reactivity with monoclonal antibody K1. Subsequent cloning studies showed that the mesothelin gene encodes a precursor protein that is processed to yield mesothelin which is attached to the cell membrane by a glycophosphatidylinositol linkage and a 31-kDa shed fragment named megakaryocyte-potentiating factor (MPF). Although it has been proposed that mesothelin may be involved in cell adhesion, its biological function is not known. A knockout mouse line that lacks mesothelin reproduces and develops normally.

Mesothelin is over expressed in several human tumors, including mesothelioma, ovarian cancer, pancreatic adenocarcinoma, lung adenocarcinoma, and cholangiocarcinoma. Mesothelin binds MUC16 (also known as CA125), indicating that the interaction of mesothelin and MUC16 may contribute to the implantation and peritoneal spread of tumors by cell adhesion. The region (residues 296-359) consisting of 64 amino acids at the N-terminus of cell surface mesothelin has been identified as the functional binding domain (named IAB) for MUC16/CA125, suggesting the mechanism of mesothelin acting as a MUC16/CA125 functional partner in cancer development.

Mesothelin is a tumor differentiation antigen that is normally present on the mesothelial cells lining the pleura, peritoneum and pericardium. Since mesothelin is overexpressed in several cancers, the protein could be exploited as tumor marker that may be detected using polypeptides of the present disclosure. For example, elevations of serum mesothelin specific to ovarian and other cancer patients may be measured using ELISA assays. Soluble mesothelin in the blood of a subject may correspond to mesothelin shed from tumor cells.

The present disclosure provides assays for blood-borne mesothelin for tumor diagnosis (e.g., as applied to asbestos-related mesothelioma). Elevated serum mesothelin was found in most patients with mesothelioma (71%) and ovarian cancer (67%). Blood mesothelin levels were correlated, with accuracy for malignant pleural mesothelioma and lung cancer (sensitivity 74% and 59%, specificity 90% and 86%, respectively for MPF and mesothelin assays). Circulating mesothelin is reported in nearly all pancreatic cancers. Increase of mesothelin-specific antibodies were also detected in the sera of about 40% of patients with mesothelioma and 42% with ovarian cancer, indicating an antibody response to mesothelin was correlated with high expression of mesothelin on tumor cells. https://en.wikipedia.org/wiki/Mesothelin-cite note-27

Further, the present disclosure provides methods for treating a subject having a neoplasia associated with over-expression of mesothelin. The methods may involve administering a polypeptide of the disclosure or antigen-binding fragment thereof, or a polynucleotide encoding the same, or a cell expressing a chimeric antigen receptor (CAR) containing the polypeptide or antigen-binding fragment thereof to the subject. In some instances, the polypeptide administered to the subject is an antibody-drug conjugate.

### VHH Antibodies

In various aspects, the disclosure provides VHH antibodies, also known as “nanobodies,” capable of binding a mesothelin antigen, as well as polypeptides containing VHH domains or polynucleotides encoding the same. In embodiments, the mesothelin bound by the VHH antibodies is associated with a disease or disorder, such as a neoplasia. In embodiments, the VHH binds an antigen associated with a target cell. In embodiments, the target cell is a neoplastic cell.

VHH domains are derived from nanobodies. Nanobodies are antibody-derived therapeutic proteins that contain the unique structural and functional properties of naturally-occurring heavy-chain antibodies. These heavy-chain antibodies contain a single variable domain (VHH) and two constant domains (CH2 and CH3). Importantly, a cloned and isolated VHH domain is a stable polypeptide harboring the full antigen-binding capacity of the original heavy-chain antibody. Nanobodies have a high homology with the VH domains of human antibodies and can be further humanized without any loss of activity. Importantly, Nanobodies have a low immunogenic potential, which has been confirmed in primate studies with Nanobody lead compounds.

Nanobodies combine the advantages of conventional antibodies with important features of small molecule drugs. Like conventional antibodies, nanobodies show high target specificity, high affinity for their target, and low inherent toxicity. However, like small molecule drugs they can inhibit enzymes and readily access receptor clefts. Furthermore, Nanobodies are stable, can be administered by means other than injection (see, e.g., WO2004041867A2, which is herein incorporated by reference in its entirety) and are easy to manufacture. Other advantages of Nanobodies include recognizing uncommon or hidden epitopes as a result of their small size, binding into cavities or active sites of protein targets with high affinity and selectivity due to their unique 3-dimensional, drug format flexibility, tailoring of half-life and ease and speed of drug discovery.

Nanobodies are encoded by single genes and are efficiently produced in almost all prokaryotic and eukaryotic hosts, e.g., E. coli (see, e.g., U.S. Pat. No. 6,765,087, which is herein incorporated by reference in its entirety), molds (for example Aspergillus or Trichoderma) and yeast (for example Saccharomyces, Kluyveromyces, Hansenula, or Pichia) (see, e.g., U.S. Pat. No. 6,838,254, which is herein incorporated by reference in its entirety).

VHHs, such as the anti-mesothelin VHHs described herein, have a number of advantages over conventional antibodies and recombinant antibody domains, including (i) they are small monomeric proteins (14 kDa) that express and fold efficiently in recombinant hosts; (ii) they are more stable to extremes of pH and temperature compared with conventional antibodies; (iii) they typically bind conformational epitopes; and (iv) they are amenable to designed multimerization which often leads to higher potencies; and (v) they offer more therapeutic versatility, such as multispecificity, thus supporting their beneficial utility in treating diseases caused by or associated with mesothelin.

The amino acid sequences of representative anti-mesothelin VHH antibodies described herein are provided in Table 1 below, and the corresponding polynucleotide sequences encoding each of the representative anti-mesothelin VHH antibodies are provided in Table 2 below. Representative embodiments of the binding regions of the anti-mesothelin VHHs include CDRs (CDR1, CDR2 and CDR3) as set forth in the sequences of representative anti-mesothelin VHHs are presented in Table 3A below. The CDR binding regions are positioned within framework (FR) regions of the VHH polypeptide (see Table 3B), which do not vary substantially in sequence between discrete anti-mesothelin VHHs and which provide a “structural scaffold” for the CDRs, which bind to mesothelin. By way of non-limiting example, the binding of CDRs within FRs to a target protein (antigen), e.g., mesothelin, may be via conformational binding or interaction, electrostatic binding interaction, hydrogen bonding, Van der Waals forces, or hydrophobic bonding, or combinations thereof, as would be appreciated by those having skill in the art.

TABLE 1
Anti-mesothelin VHH antibody amino acid sequences.

| VHH Antibody Name | Full-Length Amino Acid Sequence |
|---|---|
| M3_C7 | QVQLVESGGGLVQAGGSLRLSCVASGRTFSSYAMGWFRQSPGKER |
| | EFVAAICWSGGNTYYADSVKGRFTISGDNAKNSVYLQMNSLKPED |
| | TAVYICNERTTVLPTGAMDYWGQGTSVTVSS |
| | (SEQ ID NO: 55) |
| Ab_113415 | QVQLVESGGGLVQPGGSLRLSCAASGSMFSINAVAWYRQAPGEQR |
| | EPVAAMTSGGSTIYADSVKGRFTISRDNAKNTVYLQMSSLKPEDT |
| | AVYYCNGGWSLAMDYWGQGTSVTVS |
| | (SEQ ID NO: 56) |
| Ab_119706 | QLQLVESGGGLVQPGGSLRLSCAASGFTLDYYAIGWFRQAPGKER |
| | EGVSYISSSGGSTNYADSVKGRFTISRDNAKNTVYLQMNSLKPED |
| | TGVYSCAAAGGYGNSSRYWGQGTSVTVS |
| | (SEQ ID NO: 57) |
| Ab_119699 | QVQLVESGGGLVQPGGSLRLSCAASGSIFSVNAMGYYRQISTKQR |
| | NLVAAINTGGGSTYYADSVKGRFTISWDNANNTLFLQMNSLKSED |
| | TAVYYCAAEDFGNSKTYWGQGTLVTVS |
| | (SEQ ID NO: 58) |
| Ab_119702 | DVQLVESGGGLVQAGGSLRLSCAASGFPYFSCCMSWHRQAPGKDR |
| | ELVSSISSDGSTHYADSVRGRFTISKGNARNTLYLQMNSLKPEDT |
| | AVYFCAASPRQLGPYYAMDYWGQGTSVTVS |
| | (SEQ ID NO: 59) |
| Ab_119704 | QLQLVESGGGLVQPGGSLRLSCAASGFTLDNYAIGWFRQAPGKER |
| | EGVSSISSSGGSTNYADSVKGRFTVSRDNAKHTVYLQMNSLKPED |
| | TAVYYCAGVGDYGRRAMGQGTSVTVS |
| | (SEQ ID NO: 60) |
| Ab_119707 | EVQLVESGGGSVQAGETLRLSCTASGFTFDDSDMGWYRQAPGDEC |
| | ELVSTISSDGSTNYADSVKGRFTISQDNAKNTVYLQMTSLKPEDT |
| | AIYSCRTMASRIGCWGQGTLVTVS (SEQ ID NO: 61) |
| M1_C8 | QLQLVESGGGLVQPGGSLRLSCAASGFTLDNYAIGWFRQAPGKER |
| | EGVSSISSSGGSTNYADSVKGRFTVSRDNAKQTVYLQMNSLKPED |
| | TAVYYCAGVGDYGRRAMGQGTSVTVS (SEQ ID NO: 62) |
| M3_C3 | QLQLVESGGGLVQPGGSLRLSCAASGFTLDYFAIGWFRQAPGKER |
| | EGVSYISSSGDSTNYADTVKGRFTISRDNAKNTVYLQMNSLKPED |
| | TGVYSCAAAGGYGNSSRYWGQGTSVTVS (SEQ ID NO: 63) |
| Ab_119700 | QLQLVESGGGLVQPGGSLRLTCAASGFTLDNYAIGWFRQAPGKER |
| | EGVSSISSSGGSTNYADSVKGRFTVSRDNAKHTVYLQMNSLKPED |
| | TAVYYCAGVGDYGRRAMGQGTSVTVS (SEQ ID NO: 64) |
| M3_C6 | QVQLVESGGGLVQAGGSLRLSCAASGRNFSSYPMGWFRQAPGKER |
| | EFVAAISWSGGRTFYAASVKGRFTISRDTAKNSVYLQMNSLKPED |
| | TAVYICNERTTVLPTGAMDYWGQGTSVTVS (SEQ ID NO: 65) |
| Ab_119696 | QVQLVESGGGLVQPGGSLRLSCAASGSMFSINAVAWYRQAPGEQR |
| | EPVAAMTSGGSTIYADSVKGRFTISRDNAKNTVYLQMNSLKPEDT |
| | AVYYCNGGWSLAMDYWGQGTSVTVS (SEQ ID NO: 66) |

TABLE 2
Anti-mesothelin VHH antibody nucleotide sequences.

| VHH Antibody | |
|---|---|
| Name | Nucleotide Sequence |
| M3_C7 | CAAGTTCAGCTTGTGGAGTCAGGCGGTGGACTCGTTCAAGCCGGAGGCT |
| | CACTGCGGTTGTCTTGTGTCGCATCTGGTCGAACCTTCTCAAGTTATGC |
| | TATGGGGTGGTTCCGCCAGTCTCCTGGTAAAGAGCGGGAATTTGTCGCA |
| | GCAATTTGTTGGTCTGGTGGTAACACTTACTATGCTGATTCAGTAAAAG |
| | GTCGATTCACCATATCAGGGGATAATGCAAAAAATTCTGTCTATCTCCA |
| | AATGAACTCTCTGAAGCCTGAAGATACTGCTGTGTATATCTGTAATGAG |
| | AGGACTACGGTACTACCTACGGGTGCTATGGACTACTGGGGTCAAGGGA |
| | CCTCAGTCACCGTCTCCTCG |
| | (SEQ ID NO: 69) |
| Ab_113415 | CAAGTTCAGCTTGTCGAGAGTGGTGGAGGATTGGTGCAGCCAGGCGGAT |
| | CTCTCAGACTTTCCTGCGCCGCCAGTGGTTCTATGTTTTCCATAAATGC |
| | AGTGGCCTGGTACAGGCAAGCACCAGGCGAGCAAAGGGAACCGGTAGCC |
| | GCAATGACCTCTGGAGGTAGTACAATTTACGCTGATAGCGTCAAGGGAC |
| | GATTCACAATATCACGAGACAATGCCAAGAACACTGTCTACCTTCAAAT |
| | GAGCAGTTTGAAACCTGAGGATACAGCAGTATACTACTGCAATGGAGGA |
| | TGGTCACTGGCTATGGACTACTGGGGTCAAGGAACCTCAGTCACCGTCT |
| | CC |
| | (SEQ ID NO: 70) |
| Ab_119706 | CAGCTTCAACTGGTAGAGAGTGGTGGAGGACTTGTTCAGCCTGGAGGGT |
| | CCCTTAGACTTAGTTGCGCTGCCAGTGGGTTCACCCTGGACTACTACGC |
| | CATTGGCTGGTTTCGACAGGCCCCAGGGAAAGAGCGAGAGGGGGTCTCT |
| | TACATCAGTAGTTCCGGTGGTTCCACTAACTACGCTGATAGTGTGAAGG |
| | GGAGGTTTACTATTTCTCGGGATAATGCAAAAAATACCGTGTACCTGCA |
| | AATGAATAGCCTCAAACCAGAAGACACAGGCGTTTATTCTTGCGCAGCA |
| | GCTGGGGGCTATGGTAACTCCTCTCGCTACTGGGGTCAAGGAACCTCAG |
| | TCACCGTCTCC |
| | (SEQ ID NO: 71) |
| Ab_119699 | CAGGTTCAGCTGGTGGAGTCCGGCGGAGGTCTTGTTCAGCCCGGAGGTT |
| | CCCTGCGCCTCTCTTGTGCAGCTTCTGGTTCCATTTTCTCAGTCAATGC |
| | AATGGGCTACTACAGACAAATTTCCACAAAGCAGCGCAACTTGGTCGCC |
| | GCAATAAATACTGGTGGTGGCTCGACTTATTACGCCGATAGTGTCAAAG |
| | GACGATTTACTATAAGTTGGGACAACGCAAACAATACTTTGTTCCTCCA |
| | AATGAATAGCCTCAAATCAGAAGACACTGCCGTCTACTACTGTGCAGCA |
| | GAGGACTTTGGTAACTCGAAAACTTACTGGGGCCAAGGGACTCTGGTCA |
| | CTGTCTCT |
| | (SEQ ID NO: 72) |
| Ab_119702 | GACGTACAACTTGTGGAATCAGGTGGGGGTCTCGTTCAAGCTGGTGGGT |
| | CACTGCGGCTTTCTTGCGCTGCAAGTGGGTTCCCTTATTTTTCCTGCTG |
| | TATGAGCTGGCATCGCCAAGCTCCTGGAAAAGATCGGGAACTGGTCTCC |
| | AGTATTTCATCTGACGGGTCAACACATTACGCTGATAGCGTCAGGGGGA |
| | GGTTTACTATTTCTAAAGGAAATGCAAGAAACACACTCTACCTCCAGAT |
| | GAACAGTCTGAAGCCCGAAGACACCGCCGTGTATTTCTGTGCAGCTTCC |
| | CCTAGACAGCTCGGGCCTTACTATGCTATGGACTACTGGGGTCAAGGAA |
| | CCTCAGTCACCGTCTCC |
| | (SEQ ID NO: 73) |
| Ab_119704 | CAGCTTCAACTGGTAGAGAGTGGTGGAGGACTTGTTCAGCCTGGAGGGT |
| | CCCTTAGACTTAGTTGCGCTGCCAGTGGGTTCACCCTGGACAACTACGC |
| | CATTGGCTGGTTTCGACAGGCCCCAGGGAAAGAGCGAGAGGGGGTCTCT |
| | AGCATCAGTAGTTCCGGTGGTTCCACCAACTATGCTGATAGTGTGAAGG |
| | GGAGGTTTACTGTTTCTCGGGATAATGCAAAACATACCGTGTACCTGCA |
| | AATGAATAGCCTCAAACCAGAAGACACAGCCGTCTATTATTGCGCAGGA |
| | GTTGGGGACTACGGTAGAAGGGCGATGGGTCAAGGCACCTCAGTCACCG |
| | TCTCC |
| | (SEQ ID NO: 74) |
| Ab_119707 | GAAGTTCAGCTTGTTGAATCAGGGGGGGGAAGTGTCCAAGCTGGCGAAA |
| | CCCTTCGGCTCTCCTGCACCGCATCCGGCTTCACATTCGATGATTCAGA |
| | TATGGGCTGGTACAGGCAGGCTCCTGGTGACGAATGTGAGCTGGTATCC |
| | ACAATCTCCAGCGATGGCAGCACAAATTATGCTGACTCTGTTAAAGGGC |
| | GATTTACCATCTCTCAAGATAACGCAAAGAACACCGTGTATCTGCAGAT |
| | GACCTCATTGAAGCCAGAGGATACTGCCATATATTCATGCAGAACAATG |
| | GCCTCGAGGATTGGTTGCTGGGGCCAAGGGACTCTGGTCACTGTCTCT |
| | (SEQ ID NO: 75) |
| M1_C8 | CAGCTTCAACTGGTAGAGAGTGGTGGAGGACTTGTTCAGCCTGGAGGGT |
| | CCCTTAGACTTAGTTGCGCTGCCAGTGGATTCACCCTGGACAACTACGC |
| | CATTGGCTGGTTTCGACAGGCCCCAGGGAAAGAGCGAGAGGGGGTCTCT |
| | AGTATCAGTAGTTCCGGTGGTTCCACCAACTATGCCGATAGTGTGAAGG |
| | GGAGGTTTACTGTTTCTCGGGATAATGCAAAACAAACCGTGTACCTGCA |
| | AATGAATAGCCTCAAACCAGAAGACACAGCCGTCTATTATTGCGCAGGA |
| | GTTGGGGACTACGGTAGAAGGGCGATGGGTCAAGGCACCTCAGTCACCG |
| | TCTCC (SEQ ID NO: 76) |
| M3_C3 | CAGCTTCAACTGGTAGAGAGTGGTGGAGGACTTGTTCAGCCTGGAGGGT |
| | CCCTTAGACTTAGTTGCGCTGCCAGTGGGTTCACCCTGGACTACTTCGC |
| | CATTGGCTGGTTTCGACAGGCCCCAGGGAAAGAGCGAGAGGGGGTCTCT |
| | TACATCAGTAGTTCCGGTGATTCCACTAACTACGCTGATACTGTGAAGG |
| | GGAGGTTTACTATTTCTCGGGATAATGCAAAAAATACCGTGTACCTGCA |
| | AATGAATAGCCTCAAACCAGAAGACACAGGCGTTTATTCTTGCGCAGCA |
| | GCTGGGGGCTATGGTAACTCCTCTCGCTACTGGGGTCAAGGAACCTCAG |
| | TCACCGTCTCC (SEQ ID NO: 77) |
| Ab_119700 | CAGCTTCAACTGGTAGAGAGTGGTGGAGGACTTGTTCAGCCTGGAGGGT |
| | CCCTTAGACTTACTTGCGCTGCCAGTGGGTTCACCCTGGACAACTACGC |
| | CATTGGCTGGTTTCGACAGGCCCCAGGGAAAGAGCGAGAGGGGGTCTCT |
| | AGCATCAGTAGTTCCGGTGGTTCCACCAACTATGCTGATAGTGTGAAGG |
| | GGAGGTTTACTGTTTCTCGGGATAATGCAAAACATACCGTGTACCTGCA |
| | AATGAATAGCCTCAAACCAGAAGACACAGCCGTCTATTATTGCGCAGGA |
| | GTTGGGGACTACGGTAGAAGGGCGATGGGTCAAGGCACCTCAGTCACCG |
| | TCTCC (SEQ ID NO: 78) |
| M3_C6 | GAAGTTCAGCTTGTTGAATCAGGGGGGGGAAGTGTCCAAGCTGGCGAAA |
| | CCCTTCGGCTCTCCTGCACCGCATCCGGCTTCACATTCGATGATTCAGA |
| | TATGGGCTGGTACAGGCAGGCTCCTGGTGACGAATGTGAGCTGGTATCC |
| | ACAATCTCCAGCGATGGCAGCACAAATTATGCTGACTCTGTTAAAGGGC |
| | GATTTACCATCTCTCAAGATAACGCAAAGAACACCGTGTATCTGCAGAT |
| | GACCTCATTGAAGCCAGAGGATACTGCCATATATTCATGCAGAACAATG |
| | GCCTCGAGGATTGGTTGCTGGGGCCAAGGGACTCTGGTCACTGTCTCT |
| | (SEQ ID NO: 75) |
| Ab_119696 | CAAGTCCAGCTCGTCGAGAGTGGTGGAGGATTGGTGCAGCCAGGCGGAT |
| | CTCTCAGACTTTCCTGCGCCGCCAGTGGTTCTATGTTTTCCATAAATGC |
| | AGTGGCCTGGTACAGGCAAGCACCAGGCGAGCAAAGGGAACCGGTAGCC |
| | GCAATGACCTCTGGAGGTAGTACAATTTACGCTGATAGCGTCAAGGGAC |
| | GATTCACAATATCACGAGACAATGCCAAGAACACTGTCTACCTTCAAAT |
| | GAACAGTTTGAAACCTGAGGATACAGCAGTATACTACTGCAATGGAGGA |
| | TGGTCACTGGCTATGGACTACTGGGGTCAAGGAACCTCAGTCACCGTCT |
| | CC (SEQ ID NO: 79) |
| M1_C8 | CAGCTTCAACTGGTAGAGAGTGGTGGAGGACTTGTTCAGCCTGGAGGGT |
| | CCCTTAGACTTAGTTGCGCTGCCAGTGGATTCACCCTGGACAACTACGC |
| | CATTGGCTGGTTTCGACAGGCCCCAGGGAAAGAGCGAGAGGGGGTCTCT |
| | AGTATCAGTAGTTCCGGTGGTTCCACCAACTATGCCGATAGTGTGAAGG |
| | GGAGGTTTACTGTTTCTCGGGATAATGCAAAACAAACCGTGTACCTGCA |
| | AATGAATAGCCTCAAACCAGAAGACACAGCCGTCTATTATTGCGCAGGA |
| | GTTGGGGACTACGGTAGAAGGGCGATGGGTCAAGGCACCTCAGTCACCG |
| | TCTCC (SEQ ID NO: 76) |

TABLE 3A
Representative embodiments of complementarity determining regions of anti-
mesothelin VHH antibodies of the disclosure.

| VHH Antibody | | | |
|---|---|---|---|
| Name | CDR1 | CDR2 | CDR3 |
| M3_C7 | GRTFSSYA (SEQ | ICWSGGNT (SEQ ID | NERTTVLPTGAMDY |
| | ID NO: 1) | NO: 2) | (SEQ ID NO: 3) |
| Ab_113415 | GSMFSINA (SEQ | MTSGGST (SEQ ID | NGGWSLAMDY (SEQ ID |
| | ID NO: 4) | NO: 5) | NO: 6) |
| Ab_119706 | GFTLDYYA (SEQ | ISSSGGST (SEQ ID | AAAGGYGNSSRY (SEQ |
| | ID NO: 7) | NO: 8) | ID NO: 9) |
| Ab_119699 | GSIFSVNA (SEQ | INTGGGST (SEQ ID | AAEDFGNSKTY (SEQ |
| | ID NO: 10) | NO: 11) | ID NO: 12) |
| Ab_119702 | GFPYFSCC (SEQ | ISSDGST (SEQ ID | AASPRQLGPYYAMDY |
| | ID NO: 13) | NO: 14) | (SEQ ID NO: 15) |
| Ab_119704 | GFTLDNYA (SEQ | ISSSGGST (SEQ ID | AGVGDYGRRA (SEQ ID |
| | ID NO: 16) | NO: 8) | NO: 17) |
| Ab_119707 | GFTFDDSD (SEQ | ISSDGST (SEQ ID | RTMASRIGC (SEQ ID |
| | ID NO: 18) | NO: 14) | NO: 19) |
| M1_C8 | GFTLDNYA (SEQ | ISSSGGST (SEQ ID | AGVGDYGRRA (SEQ ID |
| | ID NO: 16) | NO: 8) | NO: 17) |
| M3_C3 | GFTLDYFA (SEQ | ISSSGDST (SEQ ID | AAAGGYGNSSRY (SEQ |
| | ID NO: 97) | NO: 99) | ID NO: 9) |
| Ab_119700 | GFTLDNYA (SEQ | ISSSGGST (SEQ ID | AGVGDYGRRA (SEQ ID |
| | ID NO: 16) | NO: 8) | NO: 17) |
| M3_C6 | GRNFSSYP (SEQ | ISWSGGRT (SEQ ID | NERTTVLPTGAMDY |
| | ID NO: 98) | NO: 100) | (SEQ ID NO: 3) |
| Ab_119696 | GSMFSINA (SEQ | MTSGGST (SEQ ID | NGGWSLAMDY (SEQ ID |
| | ID NO: 4) | NO: 5) | NO: 6) |

TABLE 3B
Framework regions of anti-mesothelin VHH antibodies of the disclosure.

| VHH Antibody | | |
|---|---|---|
| Name | FR1 | FR2 |
| M3_C7 | QVQLVESGGGLVQAGGSLRLSCVAS (SEQ | MGWFRQSPGKEREFVA |
| | ID NO: 101) | A (SEQ ID NO: |
| | | 108) |
| Ab_113415 | QVQLVESGGGLVQPGGSLRLSCAAS (SEQ | VAWYRQAPGEQREPVA |
| | ID NO: 102) | A (SEQ ID NO: |
| | | 109) |
| Ab_119706 | QLQLVESGGGLVQPGGSLRLSCAAS (SEQ | IGWFRQAPGKEREGVS |
| | ID NO: 103) | Y (SEQ ID NO: |
| | | 110) |
| Ab_119699 | QVQLVESGGGLVQPGGSLRLSCAAS (SEQ | MGYYRQISTKORNLVA |
| | ID NO: 102) | A (SEQ ID NO: |
| | | 111) |
| Ab_119702 | DVQLVESGGGLVQAGGSLRLSCAAS (SEQ | MSWHRQAPGKDRELVS |
| | ID NO: 104) | S (SEQ ID NO: |
| | | 112) |
| Ab_119704 | QLQLVESGGGLVQPGGSLRLSCAAS (SEQ | IGWFRQAPGKEREGVS |
| | ID NO: 103) | S (SEQ ID NO: |
| | | 113) |
| Ab_119707 | EVOLVESGGGSVQAGETLRLSCTAS (SEQ | MGWYRQAPGDECELVS |
| | ID NO: 105) | T (SEQ ID NO: |
| | | 114) |
| M1_C8 | QLQLVESGGGLVQPGGSLRLSCAAS (SEQ | IGWFRQAPGKEREGVS |
| | ID NO: 103) | S (SEQ ID NO: |
| | | 113) |
| M3_C3 | QLQLVESGGGLVQPGGSLRLSCAAS (SEQ | IGWFRQAPGKEREGVS |
| | ID NO: 103) | Y (SEQ ID NO: |
| | | 110) |
| Ab_119700 | QLQLVESGGGLVQPGGSLRLTCAAS (SEQ | IGWFRQAPGKEREGVS |
| | ID NO: 106) | S (SEQ ID NO: |
| | | 113) |
| M3_C6 | QVQLVESGGGLVQAGGSLRLSCAAS (SEQ | MGWFRQAPGKEREFVA |
| | ID NO: 107) | A (SEQ ID NO: |
| | | 115) |
| Ab_119696 | QVQLVESGGGLVQPGGSLRLSCAAS (SEQ | VAWYRQAPGEQREPVA |
| | ID NO: 102) | A (SEQ ID NO: |
| | | 109) |
| VHH Antibody | | |
| Name | FR3 | FR4 |
| M3_C7 | YYADSVKGRFTISGDNAKNSVYLQMNSLKPEDTA | WGQGTSVTVSS |
| | VYIC (SEQ ID NO: 116) | (SEQ ID NO: |
| | | 127) |
| Ab_113415 | IYADSVKGRFTISRDNAKNTVYLQMSSLKPEDTA | WGQGTSVTVS (SEQ |
| | VYYC (SEQ ID NO: 117) | ID NO: 128) |
| Ab_119706 | NYADSVKGRFTISRDNAKNTVYLQMNSLKPEDTG | WGQGTSVTVS (SEQ |
| | VYSC (SEQ ID NO: 118) | ID NO: 128) |
| Ab_119699 | YYADSVKGRFTISWDNANNTLFLQMNSLKSEDTA | WGQGTLVTVS (SEQ |
| | VYYC (SEQ ID NO: 119) | ID NO: 129) |
| Ab_119702 | HYADSVRGRFTISKGNARNTLYLQMNSLKPEDTA | WGQGTSVTVS (SEQ |
| | VYFC (SEQ ID NO: 120) | ID NO: 128) |
| Ab_119704 | NYADSVKGRFTVSRDNAKHTVYLQMNSLKPEDTA | MGQGTSVTVS (SEQ |
| | VYYC (SEQ ID NO: 121) | ID NO: 130) |
| Ab_119707 | NYADSVKGRFTISQDNAKNTVYLQMTSLKPEDTA | WGQGTLVTVS (SEQ |
| | IYSC (SEQ ID NO: 122) | ID NO: 129) |
| M1_C8 | NYADSVKGRFTVSRDNAKQTVYLQMNSLKPEDTA | MGQGTSVTVS (SEQ |
| | VYYC (SEQ ID NO: 123) | ID NO: 130) |
| M3_C3 | NYADTVKGRFTISRDNAKNTVYLQMNSLKPEDTG | WGQGTSVTVS (SEQ |
| | VYSC (SEQ ID NO: 124) | ID NO: 128) |
| Ab_119700 | NYADSVKGRFTVSRDNAKHTVYLQMNSLKPEDTA | MGQGTSVTVS (SEQ |
| | VYYC (SEQ ID NO: 121) | ID NO: 130) |
| M3_C6 | FYAASVKGRFTISRDTAKNSVYLQMNSLKPEDTA | WGQGTSVTVS (SEQ |
| | VYIC (SEQ ID NO: 125) | ID NO: 128) |
| Ab_119696 | IYADSVKGRFTISRDNAKNTVYLQMNSLKPEDTA | WGQGTSVTVS (SEQ |
| | VYYC (SEQ ID NO: 126) | ID NO: 128) |

In various embodiments, the FR1 of a VHH antibody of the disclosure contains the following amino acid sequence:
X₁X₂QLVESGGGX₃VQX₄GX₅X₆LRLX₇CX₈AS (SEQ ID NO: 20), wherein
   X₁ is E, D, or Q,
   X₂ is V or L,
   X₃ is L or S,
   X₄ is A or P,
   X₅ is E or G,
   X₆ is S or T,
   X₇ is S or T, and
   X₈ is A, T, or V.

In various embodiments, the FR2 of a VHH antibody of the disclosure contains the following amino acid sequence:
XₐX_{b}X₁₃X₁₄RQX₁₅X₁₆X₁₇X₁₈X₁₉X₂₀X₂₁X₂₂VX₂₃X_{c}, wherein
   Xₐ is I, M, or V,
   X_{b} is A, G, or S,
   X₁₃ is W or Y,
   X₁₄ is F, H, or Y,
   X₁₅ is A, I, or X,
   X₁₆ is P or S,
   X₁₇ is G or T,
   X₁₈ is D, K, or E,
   X₁₉ is D, E, or Q,
   X₂₀ is C or R,
   X₂₁ is E or N,
   X₂₂ is F, G, L, or P,
   X₂₃ is S or A, and
   Xₑ is A, S, T, or Y.

In various embodiments, the FR3 of a VHH antibody of the disclosure contains the following amino acid sequence:
X_{d}YAXₑX_{f}VX_{g}GRFTX₂₄SX₂₅X₂₆X₂₇AX₂₈X₂₉X₃₀X₃₁X₃₂LQMX₃₃SLKX₃₄EDT X₃₅X₃₆YX₃₇C (SEQ ID NO: 21), wherein
   X_{d} is F, H, I, N, or Y,
   Xₑ is A or D,
   X_{f} is S or T,
   X_{g} is K or R,
   X₂₄ is I or V,
   X₂₅ is G, K, R, Q, or W,
   X₂₆ is D or G,
   X₂₇ is N or T,
   X₂₈ is K, N, or R,
   X₂₉ is N, H, or Q,
   X₃₀ is T or S,
   X₃₁ is V or L,
   X₃₂ is F or Y,
   X₃₃ is N, S, or T,
   X₃₄ is P or S,
   X₃₅ is A or G,
   X₃₆ is I or V, and
   X₃₇ is F, I, S, or Y.

In various embodiments, the FR4 of a VHH antibody of the disclosure contains the following amino acid sequence:
X₄₀GQGTX₄₁VTVX₄₂X₄₃ (SEQ ID NO: 22), wherein
   X₄₀ is M or W,
   X₄₁ is L or S,
   X₄₂ is null or S, and
   X₄₃ is null or S.

The CDRs of the anti-mesothelin VHH polypeptides described herein may vary in amino acid sequence length. By way of nonlimiting example, CDR1 of the anti-mesothelin VHH polypeptides as described herein may comprise from about 5 to about 12 (e.g., 5 or 8) amino acid residues; CDR2 may comprise from about 7 to about 20 (e.g., 16, 17, or 8) amino acid residues; and CDR3 may comprise from about 5 to about 25 (e.g., 7-13, 15-24, or 16) amino acid residues. It will be appreciated by one skilled in the art that number of amino acids that constitute a CDR is not necessarily precise. In some cases, an amino acid residue, or 2 or 3 amino acid residues, at one end or both ends of a given CDR may be considered as part of the CDR or as part of the neighboring FR region. The CDR regions of representative anti-mesothelin VHH antibody polypeptides described herein are presented in Table 3A. The anti-mesothelin VHH antibodies described herein demonstrate the CDR diversity that is selected during affinity maturation of mesothelin binding polypeptides in the same animal. Despite such CDR diversity, the mesothelin binding VHHs generated as described herein show detectable binding to mesothelin. The anti-mesothelin VHH polypeptides demonstrate significant binding to the mesothelin antigen, despite some variation among the CDR sequences in the context of their framework regions.

In view of the representative anti-mesothelin VHH amino acid sequences listed in Table 1, it will be appreciated by one skilled in the art that individual VHH polypeptides, (e.g., of from about 105 to about 140 amino acids in length and comprising 3 CDRs and 4 FR regions), which comprise at least about or equal to 85%, or 88%, or greater identity in amino acid sequence bind to mesothelin antigen. In an embodiment, at least about or equal to 85%, 86%, 87%, 88%, 89%, 90%, 91%, 92%, 93%, 94%, 95%, 96%, 97%, 98%, 99%, or 100% amino acid sequence identity is tolerated among the anti-mesothelin VHHs without adversely affecting or eliminating binding of the VHH polypeptides to the mesothelin antigen. In an embodiment, such amino acid sequence variation among the anti-mesothelin VHH polypeptides is tolerated in the CDRs of the VHH polypeptides without adversely affecting binding of the VHHs to mesothelin. In a particular embodiment, the amino acid sequence variations between or among anti-mesothelin VHHs encompass one or more conservative amino acid substitutions or changes in a VHH amino acid sequence. In an embodiment, the one or more conservative amino acid substitutions or changes in a VHH amino acid sequence occur in one or more CDR sequences of the VHH, in one or more FR sequences of the VHH, or in CDR and FR sequences of the VHH.

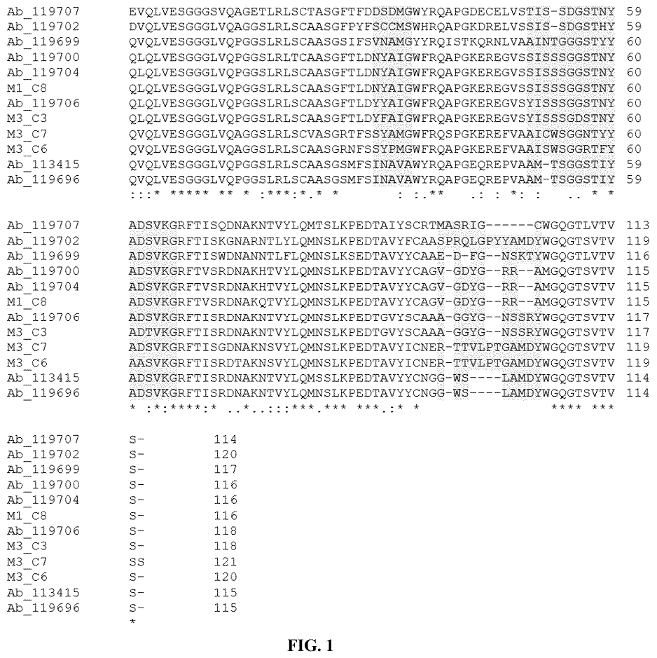

The three CDRs of the anti-mesothelin VHH polypeptides are arranged or positioned in the context of four FR regions as follows: FR1-CDR1-FR2-CDR2-FR3-CDR3-FR4, in which FR1 to FR4 refer to the framework regions 1-4, respectively, and in which CDR1 to CDR3 refer to the complementarity determining regions 1-3, respectively. An alignment of anti-mesothelin VHHs, all of which specifically bind to mesothelin protein antigen, demonstrates the extensive similarities among the sequences of each of the FRs (FR1, FR2, FR3 and FR4) found in the different mesothelin-binding VHH polypeptides (FIG. 1). Similar to the FRs in conventional antibody polypeptides, the respective FRs (FR1, FR2, FR3 and FR4) of the anti-mesothelin VHH polypeptides described herein are highly similar in sequence among different mesothelin-binding VHHs that were generated. Accordingly, provided are anti-mesothelin VHH polypeptides comprising CDR1-3, in the structural context of FR1-4, that bind to mesothelin protein, or to suitable fragments of the mesothelin protein, as well as polypeptides that comprise or consist essentially of one or more of the anti-mesothelin VHHs and/or mesothelin binding fragments thereof (e.g., a chimeric antigen receptor (CAR) polypeptide).

In addition, the FRs of the mesothelin-binding VHHs described herein are highly or essentially similar in sequence to the FRs of VHHs produced in camelid animals, such as alpacas, camels, llamas, and the like. As they provide structural and conformational support for the CDRs of VHH polypeptides, the FRs and the FR1, FR2, FR3 and FR4 regions among camelid VHH polypeptides generally share significant sequence identity. See, e.g., A. M. Vattekatte et al., March, 2020, Peer J., 6(8):e8408. DOI: 10.7717/peerj.8408 and L. S. Mitchell and L. J. Colwell, 2018, Proteins, 86(7): 697-706).

Table 3B presents the amino acid sequences of the four framework regions, i.e., FR1, FR2, FR3 and FR4, respectively, of representative anti-mesothelin VHH polypeptides described herein; and FIG. 1 presents a multiple sequence alignment of the amino acid sequences of the VHH polypeptides with CDR regions highlighted in grey. The alignment of the sequences supports substantial similarity among the structural FRs of the anti-mesothelin camelid VHH antibodies described herein.

In embodiments, in cases in which a FR (or CDR) amino acid residue in a VHH polypeptide may be one of several alternative amino acid residues, the alternative amino acid residues will frequently share similar characteristics or properties, e.g., hydrophobicity, polarity, and/or charge. A conservative replacement (also called a conservative substitution) is an amino acid replacement or substitution in a polypeptide or region thereof that changes a given amino acid residue to a different amino acid residue with similar biochemical properties, such as charge, hydrophobicity, and/or size. By way of non-limiting example, the below Table 4 presents amino acids and their 1-letter codes categorized into six main classes based on their structure and the general chemical characteristics of their side chains (R groups).

TABLE 4
Classes of amino acids based on structural and
chemical characteristics of their side chains.

| Amino Acids | Class |
|---|---|
| Glycine (G), Alanine (A), Valine (V), Leucine | Aliphatic |
| (L), Isoleucine (I) | |
| Serine (S), Cysteine I, Selenocysteine (U), | Hydroxyl or sulfur/selenium |
| Threonine (T), Methionine (M) | containing |
| Proline (P) | Cyclic |
| Phenylalanine (F), Tyrosine (Y), Tryptophan | Aromatic |
| (W) | |
| Histidine (H), Lysine (K), Arginine (R) | Basic |
| Aspartate (D), Glutamate (E), Asparagine (N), | Acidic and amides thereof |
| Glutamine (Q) | |

In embodiments, an amino acid within an FR or CDR of a VHH antibody of the disclosure is substituted with another amino acid from the same class indicated in Table 4.

In an embodiment, amino acid sequence substitutions or changes in an anti-mesothelin VHH polypeptide relative to another anti-mesothelin VHH polypeptide comprise conservative amino acid substitutions or changes such that a given amino acid residue is substituted with or replaced by a different amino acid residue with similar biochemical properties, such as charge, hydrophobicity, and/or size. In an embodiment, sequence variation between or among anti-mesothelin VHH polypeptides results from one or more conservative amino acid changes and account for the percent sequence variation, e.g., 85%, 86%, 87%, 88%, 90%, 91%, 92%, 93%, 94%, 95%, 96%, 97%, 98%, or 99% sequence variation.

In some embodiments, the VHHs as described herein are humanized using methods and techniques practiced by those having skill in the art. (See, e.g., U.S. Pat. Nos. 8,975,382 and 10,550,174, the contents of which are incorporated by reference herein).

The anti-mesothelin VHH antibodies described herein have widespread application (e.g., as antigen binding domains of chimeric antigen receptor polypeptides). In embodiments, the disclosure provides polynucleotides that encode operably linked modular components that constitute the described anti-mesothelin VHHs. In embodiments, the anti-mesothelin VHHs are recombinantly produced. In embodiments, the anti-mesothelin VHHs encompass the proteins (polypeptides) encoded by the polynucleotides. In embodiments, the polynucleotide is DNA, cDNA, RNA, mRNA, or the like. In an embodiment, the anti-mesothelin VHHs may be humanized or codon-optimized using methods practiced by those having skill in the art.

Suitable methods of producing or isolating antibody fragments having the requisite binding specificity and affinity for binding to an epitope tag include for example, methods which select recombinant antibody from a library or by PCR (e.g., U.S. Pat. No. 5,455,030 and U.S. Pat. No. 7,745,587 each of which is incorporated by reference herein in its entirety).

Functional fragments of antibodies, including fragments of chimeric, humanized, primatized, veneered, or single chain antibodies, can also be produced. Functional fragments or portions of the foregoing antibodies include those which are reactive with the mesothelin protein. For example, antibody fragments capable of binding to mesothelin or a portion thereof, include, but not limited to, scFvs, Fabs, VHHs, Fv, Fab, ‘ab’ and F‘ab’)₂. Such fragments can be produced by enzymatic cleavage or by recombinant techniques. For instance, papain or pepsin cleavage are used generate Fab or F‘ab’)₂ antibody fragments, respectively. Antibody fragments are produced in a variety of truncated forms using antibody-encoding genes in which one or more stop codons has been introduced upstream of the natural stop site. For example, a chimeric gene encoding a F‘ab’)₂ heavy chain peptide portion can be designed to include DNA sequences encoding the CH₁ peptide domain and hinge region of an immunoglobulin heavy chain.

### Epitope Tags

In certain embodiments, an anti-mesothelin VHH antibody includes a single epitope tag (single tag sequence) or multiple tags (multiple tag sequences), to which anti-tag antibodies specifically bind. Such epitope tags, which are specifically bindable by the anti-epitope tag antibodies, are useful in detecting VHHs bound to mesothelin protein antigen. The epitope tags may be placed at the amino terminus, carboxy terminus, or internally within a VHH molecule. Such tags and/or anti-tag antibodies are described for example, in (U.S. Pat. Nos. 8,349,326; 9,023,352, WO 2019/094095A1) and U.S. Pat. Nos. 7,943,345; 8,114,634 and 8,865,871), the contents of which are incorporated herein by reference in their entireties. By way of illustrative example, peroxidase labeled antibodies that bind the anti-O-tag antibody may be used to detect these anti-tag antibodies in assays in which samples are incubated with goat anti-O-tag-HRP conjugated antibody (Bethyl labs) diluted 1:5000 in blocking solution for 1 hour at RT with rocking and were washed as above before adding TMB microwell peroxidase substrate (KPL) to develop (incubated for 10-40 min). Development was stopped with 1M H₂SO₄ and the plates were read at 450 nm on an ELx808 Ultra Microplate Reader (Bio-Tek instruments), (Mukherjee, J. et al., 2012, PloS ONE 7:e29941).

Chimeric Antigen Receptors (CAR) and CAR-T Cells The disclosure provides immune cells that express chimeric antigen receptors (CARs) containing anti-mesothelin VHH domains (e.g., M3_C7, Ab_113415, Ab_119706, Ab 119699, Ab 119702, Ab 119704, Ab_119707, M1_C8, M3_C3, Ab_119700, M3_C6, or Ab_119696) as antigen binding domains. Non-limiting examples of CARs include those described in Larson and Maus, “Recent advances and discoveries in the mechanisms and functions of CAR-T cells,” Nature Reviews Cancer, 21:145-161 (2021), the disclosure of which is incorporated herein by reference in its entirety for all purposes. Modification of immune cells to express a chimeric antigen receptor can enhance an immune cell's immunoreactive activity. In embodiments, the chimeric antigen receptor has an affinity for an epitope on mesothelin. In some cases, the antigen is associated with an altered fitness of an organism. For example, the chimeric antigen receptor can have an affinity for an epitope mesothelin expressed in a neoplastic cell. Because the CAR-T cells can act independently of major histocompatibility complex (MHC), activated CAR-T cells can kill the neoplastic cell expressing the antigen.

Some embodiments of the methods provided herein involve autologous immune cell immunotherapy, wherein immune cells are obtained from a subject having a disease or altered fitness characterized by cancerous or otherwise altered cells expressing a surface marker. The obtained immune cells are genetically modified to express a chimeric antigen receptor and are effectively redirected against specific antigens. Thus, in some embodiments, immune cells are obtained from a subject in need of CAR-T immunotherapy. In some embodiments, these autologous immune cells are cultured and modified shortly after they are obtained from the subject. In other embodiments, the autologous cells are obtained and then stored for future use. This practice may be advisable for individuals who may be undergoing parallel treatment that will diminish immune cell counts in the future. In allogeneic immune cell immunotherapy, immune cells can be obtained from a donor other than the subject who will be receiving treatment. In some embodiments, immune cells are obtained from a healthy subject or donor and are genetically modified to express a chimeric antigen receptor and are effectively redirected against specific antigens. The immune cells, after modification to express a chimeric antigen receptor, are administered to a subject for treating a neoplasia (e.g., a cancer). In some embodiments, immune cells to be modified to express a chimeric antigen receptor can be obtained from pre-existing stock cultures of immune cells.

Immune cells and/or immune effector cells can be isolated or purified from a sample collected from a subject or a donor using standard techniques known in the art. For example, immune effector cells can be isolated or purified from a whole blood sample by lysing red blood cells and removing peripheral mononuclear blood cells by centrifugation. The immune effector cells can be further isolated or purified using a selective purification method that isolates the immune effector cells based on cell-specific markers such as CD25, CD3, CD4, CD8, CD28, CD45RA, or CD45RO. In one embodiment, CD4⁺ is used as a marker to select T cells. In one embodiment, CD8⁺ is used as a marker to select T cells. In one embodiment, CD4⁺ and CD8⁺ are used as a marker to select regulatory T cells.

One technique for isolating or purifying immune effector cells is flow cytometry. In fluorescence activated cell sorting a fluorescently labelled antibody with affinity for an immune effector cell marker is used to label immune effector cells in a sample. A gating strategy appropriate for the cells expressing the marker is used to segregate the cells. For example, T lymphocytes can be separated from other cells in a sample by using, for example, a fluorescently labeled antibody specific for an immune effector cell marker (e.g., CD4, CD8, CD28, CD45) and corresponding gating strategy. In one embodiment, a CD4 gating strategy is employed. In one embodiment, a CD8 gating strategy is employed. In one embodiment, a CD4 and CD8 gating strategy is employed. In some embodiments, a gating strategy for other markers specific to an immune effector cell is employed instead of, or in combination with, the CD4 and/or CD8 gating strategy.

The immune effector cells contemplated in the disclosure are effector T cells or NK cells. In some embodiments, the effector T cell is a naïve CD8⁺ T cell, a cytotoxic T cell, a natural killer T (NKT) cell, a natural killer cell, a gammadelta T cell (γδ cell), or a regulatory T (Treg) cell. In some embodiments, the effector T cells are thymocytes, immature T lymphocytes, mature T lymphocytes, resting T lymphocytes, or activated T lymphocytes. In some embodiments the immune effector cell is a CD4⁺ CD8⁺ T cell or a CD4⁻ CD8⁻ T cell. In some embodiments the immune effector cell is a T helper cell. In some embodiments the T helper cell is a T helper 1 (Th1), a T helper 2 (Th2) cell, or a helper T cell expressing CD4 (CD4+ T cell). In some embodiments, the immune effector cell is any other subset of T cells.

Chimeric antigen receptors (CARs) as contemplated in the present disclosure comprise an extracellular binding domain (e.g., a VHH domain), a transmembrane domain (e.g., a transmembrane domain derived from a CD28, CD8, or CD3 polypeptide), and an intracellular domain (e.g., an intracellular domain containing a CD3ζ signaling domain, and/or a co-stimulation domain derived from a CD28, 41BB, OX40, or CD27 polypeptide). Binding of an antigen to the extracellular binding domain can activate the CAR-T cell and generate an effector response, which includes CAR-T cell proliferation, cytokine production, and other processes that lead to the death of the antigen expressing cell. In some embodiments of the present disclosure, the chimeric antigen receptor further comprises a linker (e.g., a hinge domain derived from a CD8 CD28, CD4, or IgG polypeptide). In some cases, the CAR comprises a signal peptide, such as a signal peptide derived from a CD28 or GCSF signal peptide.

In various embodiments, the CAR specifically binds any antigen that can be targeted by a chimeric antigen receptor (CAR), such as mesothelin. Further non-limiting examples of antigens that can be bound by a CAR of the present disclosure include those described in Xu, et al. “The development of CAR design for tumor CAR-T cell therapy,” Oncotarget, 9(17) doi: 10.18632/oncotarget.24179, the disclosure of which is incorporated herein by reference in its entirety for all purposes.

Chimeric antigen receptors, or any polypeptide of the present disclosure, can be delivered to an immune cell using a polynucleotide encoding the chimeric antigen receptor or polypeptide. For example, immune cells obtained from a subject may be transduced with a nucleic acid vector encoding the chimeric antigen receptor. The vector may then be used to transduce recipient immune cells so that these cells will then express the chimeric antigen receptor. Efficient means of transducing immune cells include transfection and transduction. Such methods are well known in the art. For example, applicable methods for delivery the nucleic acid molecule encoding the chimeric antigen receptor (and the nucleic acid(s) encoding the base editor) can be found in International Application No. PCT/US2009/040040 and U.S. Pat. Nos. 8,450,112; 9,132,153; and 9,669,058, each of which is incorporated herein in its entirety. Additionally, those methods and vectors described herein for delivering the nucleic acid encoding the base editor are applicable to delivering the nucleic acid encoding the chimeric antigen receptor.

### Extracellular Binding Domain

The chimeric antigen receptors of the disclosure include an extracellular binding domain (e.g., an anti-mesothelin VHH domain, such as M3_C7, Ab_113415, Ab_119706, Ab 119699, Ab 119702, Ab 119704, Ab_119707, M1_C8, M3_C3, Ab_119700, M3_C6, and Ab_119696). The extracellular binding domain of a chimeric antigen receptor contemplated herein comprises an amino acid sequence of an antibody (e.g., a VHH antibody), or an antigen binding fragment thereof, that has an affinity for mesothelin.

In some embodiments the chimeric antigen receptor contains an amino acid sequence of an antibody (e.g., a VHH antibody such as M3_C7, Ab_113415, Ab_119706, Ab_119699, Ab 119702, Ab 119704, Ab_119707, M1_C8, M3_C3, Ab 119700, M3_C6, and Ab_119696). In some embodiments, the chimeric antigen receptor contains the amino acid sequence of an antigen binding fragment of an antibody. The antibody (or fragment thereof) portion of the extracellular binding domain recognizes and binds to an epitope of mesothelin. In some embodiments, the antibody fragment portion of a chimeric antigen receptor is a VHH domain. In other embodiments, the antibody fragment portion of a chimeric antigen receptor is a multichain variable fragment, which can comprise more than one extracellular binding domains and therefore bind to more than one antigen simultaneously. In a multiple chain variable fragment embodiment, a hinge region may separate the different variable fragments, providing necessary spatial arrangement and flexibility.

In embodiments, the antigen-binding portion of a chimeric antigen receptor comprises complementarity determining regions (e.g., CDR1, CDR2, and CDR3 regions) that are responsible for the antibody's affinity for a particular antigen. Thus, antibodies that recognize different antigens comprise different complementarity determining regions.

In some embodiments, the antigen recognized and bound by the extracellular domain is a protein or peptide, a nucleic acid, a lipid, or a polysaccharide. Antigens can be heterologous, such as those expressed in a pathogenic bacteria or virus. Antigens can also be synthetic; for example, some individuals have extreme allergies to synthetic latex and exposure to this antigen can result in an extreme immune reaction. In some embodiments, the antigen is autologous, and is expressed on a diseased or otherwise altered cell. For example, in some embodiments, the antigen is expressed in a neoplastic cell.

### Transmembrane Domain

The chimeric antigen receptors of the disclosure include a transmembrane domain. The transmembrane domain of the chimeric antigen receptors described herein spans the CAR-T cell's or CAR NT cell's lipid bilayer cellular membrane and separates the extracellular binding domain and the intracellular signaling domain. In some embodiments, this domain is derived from other receptors having a transmembrane domain, while in other embodiments, this domain is synthetic. In some embodiments, the transmembrane domain may be derived from a non-human transmembrane domain and, in some embodiments, humanized. By “humanized” is meant having the sequence of the nucleic acid encoding the transmembrane domain optimized such that it is more reliably or efficiently expressed in a human subject. In some embodiments, the transmembrane domain is derived from another transmembrane protein expressed in a human immune effector cell.

### Intracellular Signaling Domain

The chimeric antigen receptors of the disclosure include an intracellular signaling domain. The intracellular signaling domain is the intracellular portion of a protein expressed in a T cell or NK cell that transduces an effector function signal (e.g., an activation signal) and directs the effector cell to perform a specialized function. Effector cell activation can be induced by a number of factors, including binding of cognate antigen to the chimeric antigen receptor on the surface of effector cell and/or binding of cognate ligand to costimulatory molecules on the surface of the cell. An effector cell co-stimulatory molecule is a cognate binding partner on an immune effector cell that specifically binds with a co-stimulatory ligand, thereby mediating a co-stimulatory response by the effector cell, such as, but not limited to, proliferation. Co-stimulatory molecules include, but are not limited to, an MHC class I molecule. Activation of an effector cell leads to immune response, Such as effector cell proliferation and differentiation (see, e.g., Smith-Garvin et al., Annu. Rev. Immunol., 27:591-619, 2009). Exemplary effector cell (e.g., T cells) signaling domains are known in the art.

The intracellular signaling domain of the chimeric antigen receptor contemplated herein comprises a primary signaling domain. In some embodiments, the chimeric antigen receptor comprises the primary signaling domain and a secondary, or co-stimulatory, signaling domain.

### Polynucleotides and Vectors

In some cases, more than one anti-mesothelin-binding VHH antibody (i.e., anti-mesothelin VHH) is coupled or linked (e.g., covalently linked) to other sequences, e.g., a leader amino acid sequence, domains of a chimeric antigen receptor, one or more spacer or linker (flexible spacer or linker) amino acid sequences, or one or more epitope tag amino acid sequences. In an embodiment, a polynucleotide molecule, such as a recombinant or isolated polynucleotide molecule, encodes a polypeptide containing an anti-mesothelin VHH polypeptide (e.g., a VHH antibody or a chimeric antigen receptor). In an embodiment, the polynucleotide encodes a fragment or portion of the anti-mesothelin VHH, where the fragment or portion maintains mesothelin binding activity. The polynucleotide sequences encoding representative anti-mesothelin VHH antibodies as described herein are set forth in Table 2.

In an embodiment, an anti-mesothelin VHH can be humanized, i.e., modified to increase its similarity to antibodies or antibody variants produced naturally in humans, using techniques known and practiced in the art. Briefly and by way of nonlimiting example, a humanized antibody can be generated by inserting the appropriate CDR coding sequences (e.g‘, ‘do’ or’ sequences that are responsible for the desired binding properties) into a human antibo”y “scaff”ld” (e.g‘, ‘accep’ or’ sequences) comprising essentially invariant framework region (FR) sequences (FRs). In embodiments, the CDRs of the anti-mesothelin VHH antibodies described herein may be inserted into FRs, which provide the structural scaffold that allows the CDRs to bind to mesothelin. Recombinant DNA methods using an appropriate vector and expression in mammalian cells are employed and routinely practiced in the art to achieve the production of recombinant humanized antibodies.

In an embodiment, the polynucleotide encodes a mesothelin-binding VHH molecule having binding function, or a functional binding portion thereof. In embodiments, antibody fragments, microproteins, darpins, anticalins, peptide mimetic molecules, aptamers, synthetic molecules, etc. can be linked to the anti-mesothelin VHH binding molecule.

In an embodiment, an anti-mesothelin VHH can be modified, for example, by attachment (e.g., either directly or indirectly via a linker or spacer) to another agent (e.g., a detectable label, a cytotoxic drug, and/or another polypeptide). Accordingly, a polynucleotide (e.g., DNA) that encodes one anti mesothelin VHH is joined (in reading frame) with a polynucleotide encoding a second polypeptide, and so on. In certain embodiments, additional amino acids are encoded within the polynucleotide between the anti-mesothelin VHH and other polypeptides so as to produce an unstructured region (e.g., a flexible spacer) that separates the anti-mesothelin VHH from the other polypeptides to better promote independent folding of each polypeptide into its active or functional conformation or shape. Commercially available techniques for fusing proteins (or their encoding polynucleotides) may be employed to recombinantly join or couple polypeptide sequences to one another.

The compositions and methods described herein in various embodiments include an isolated polynucleotide sequence or an isolated polynucleotide molecule that encodes a polypeptide (e.g., anti-mesothelin VHH or a chimeric antigen receptor containing an anti-mesothelin VHH domain of the disclosure). Accordingly, in some embodiments, the isolated polynucleotide sequence or isolated polynucleotide molecule comprises or consists of a polynucleotide sequence that encodes a polypeptide molecule (anti-mesothelin VHH) having an amino acid sequence listed in Table 1, or a functional portion thereof, as described herein. In some embodiments, the isolated polynucleotide sequence or isolated polynucleotide molecule comprises or consists of a polynucleotide sequence listed in Table 2. In an embodiment, a composition comprises a combination of the isolated polynucleotide sequences or isolated polynucleotide molecules.

Also encompassed by the present disclosure are polynucleotide sequences, DNA or RNA, which are substantially complementary to the DNA sequences encoding the polypeptides described herein, and which specifically hybridize with these DNA sequences under conditions of stringency known to those of skill in the art. As referred to herein, substantially complementary means that the nucleotide sequence of the polynucleotide need not reflect the exact sequence of the original encoding sequences, but must be sufficiently similar in sequence to permit hybridization with a nucleic acid sequence under high stringency conditions. For example, non-complementary bases can be interspersed in a nucleotide sequence, or the sequences can be longer or shorter than the polynucleotide sequence, provided that the sequence has a sufficient number of bases complementary to the sequence to allow hybridization thereto. Conditions for stringency are described, e.g., in Ausubel, F. M., et al., Current Protocols in Molecular Biology, (Current Protocol, 1994), and Brown, et al., Nature, 366:575 (1993); and further defined in conjunction with certain assays.

Vectors and plasmids containing one or more of the polynucleotide molecules encoding the anti-mesothelin VHH amino acid sequences of Table 1, or a functional portion thereof, are provided. Vectors and plasmids containing one or more of the polynucleotide molecules of Table 2 are also provided. Suitable vectors for use in eukaryotic and prokaryotic cells are known in the art and are commercially available or readily prepared by the skilled practitioner in the art. Additional vectors can also be found, for example, in Ausubel, F. M., et al., Ibid. and in Sambrook et al”, “Molecular Cloning: A Laboratory Manu“1,” 2nd ED. (1989), and other editions.

Uses of plasmids, vectors or viruses (viral vectors) containing polynucleotides encoding the anti-mesothelin VHHs as described herein include generation of mRNA or protein in vitro or in vivo. In related embodiments, host cells transformed with the plasmids, vectors, or virus vectors are provided, as described above. Nucleic acid molecules can be inserted into a construct (such as a prokaryotic expression plasmid, a eukaryotic expression vector, or a viral vector construct, which can, optionally, replicate and/or integrate into a recombinant host cell by known methods. The host cell can be a eukaryote or prokaryote and can include, for example and without limitation, yeast (such as Pichia pastoris or Saccharomyces cerevisiae), bacteria (such as E. coli, or Bacillus subtilis), animal cells or tissue (CHO or COS cells), insect Sf9 cells (such as baculoviruses infected SF9 cells), or mammalian cells (somatic or embryonic cells, Human Embryonic Kidney (HEK) cells, Chinese hamster ovary (CHO) cells, HeLa cells, human 293 cells (Expi293F), and monkey COS-7 cells). Suitable host cells also include a mammalian cell, a bacterial cell, a yeast cell, an insect cell, a plant cell, or an algal cell.

In another aspect, an RNA polynucleotide, in particular, mRNA, encodes a polypeptide as described herein. mRNA encoding the polypeptides may contain a 5′ cap structure, a 5′ UTR, an open reading frame, a 3′ UTR and poly-A sequence followed by a C30 stretch and a histone stem loop sequence (Thess, A. et al., 2015, Mol Ther, 23(9):1456-1464; Thran, M. et al., 2017, EMBO Molecular Medicine, DOI: 10.15252/emmm.201707678). Sequences may be codon-optimized for human use using techniques and protocols known and used by those skilled in the art. In an embodiment, the mRNA sequences do not include chemically modified bases. mRNAs encoding the anti-mesothelin VHHs thereof as described herein may be capped enzymatically or further polyadenylated for in vivo studies/use. In an embodiment, a polypeptide of the disclosure is encoded by a mRNA molecule. In an embodiment, the mRNA may be delivered to or introduced into a cell.

Expression of proteins, which normally have a shortened serum half-life, by encoding mRNA, particularly sequence optimized, unmodified mRNA, advantageously prolongs the bioavailability of these proteins for in vivo activity. (see, e.g., K. Kariko et al, 2012, Mol. Ther., 20:948-953; Thess, A. et al., 2015, Mol Ther, 23(9):1456-1464;).

### Recombinant Polypeptide Expression

In general, polypeptides of the disclosure (e.g., VHH antibodies) may be produced by transformation of a suitable host cell with all or part of a polypeptide-encoding nucleic acid molecule or fragment thereof in a suitable expression vehicle.

Those skilled in the field of molecular biology will understand that any of a wide variety of systems may be used to express a recombinant protein. The precise host cell used is not critical to the various aspects and embodiments of the disclosure. A polypeptide of the disclosure may be produced in a prokaryotic host (e.g., E. coli) or in a eukaryotic host (e.g., an immune cell, such an immune effector cell (e.g., a T cell) Saccharomyces cerevisiae, insect cells, e.g., Sf21 cells, or mammalian cells, e.g., NIH 3T3, HeLa, or COS cells). Such cells are available from a wide range of sources (e.g., the American Type Culture Collection, Rockland, Md.; also, see, e.g., Ausubel et al., supra). The method of transformation or transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual (P. H. Pouwels et al., 1985, Supp. 1987).

A variety of expression systems exist for the production of the polypeptides (e.g., VHH antibodies or chimeric antigen receptors) of the disclosure. Expression vectors useful for producing such polypeptides include, without limitation, chromosomal, episomal, and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof.

One particular bacterial expression system for polypeptide production is the E. coli pET expression system (Novagen, Inc., Madison, Wis). According to this expression system, DNA encoding a polypeptide is inserted into a pET vector in an orientation designed to allow expression. Since the gene encoding such a polypeptide is under the control of the T7 regulatory signals, expression of the polypeptide is achieved by inducing the expression of T7 RNA polymerase in the host cell. This is typically achieved using host strains that express T7 RNA polymerase in response to IPTG induction. Once produced, recombinant polypeptide is then isolated according to standard methods known in the art, for example, those described herein.

Another bacterial expression system for polypeptide production is the pGEX expression system (Pharmacia). This system employs a GST gene fusion system that is designed for high-level expression of genes or gene fragments as fusion proteins with rapid purification and recovery of functional gene products. The protein of interest is fused to the carboxyl terminus of the glutathione S-transferase protein from Schistosoma japonicum and is readily purified from bacterial lysates by affinity chromatography using Glutathione Sepharose 4B. Fusion proteins can be recovered under mild conditions by elution with glutathione. Cleavage of the glutathione S-transferase domain from the fusion protein is facilitated by the presence of recognition sites for site-specific proteases upstream of this domain. For example, proteins expressed in pGEX-2T plasmids may be cleaved with thrombin; those expressed in pGEX-3X may be cleaved with factor Xa.

Once the recombinant polypeptide of the disclosure is expressed, it can be isolated, e.g., using affinity chromatography. In one example, an antibody (e.g., produced as described herein) raised against an antigen of the disclosure may be attached to a column and used to isolate the recombinant polypeptide. Lysis and fractionation of polypeptide-harboring cells prior to affinity chromatography may be performed by standard methods.

Once isolated, a recombinant protein can, if desired, be further purified, e.g., by high performance liquid chromatography (see, e.g., Fisher, Laboratory Techniques In Biochemistry and Molecular Biology, eds., Work and Burdon, Elsevier, 1980). Polypeptides of the disclosure, particularly short peptide fragments, can also be produced by chemical synthesis (e.g., by the methods described in Solid Phase Peptide Synthesis' 2nd ed., 1984 The Pierce Chemical Co., Rockford, Ill.). These general techniques of polypeptide expression and purification can also be used to produce and isolate useful peptide fragments or analogs (described herein).

### Compositions

Provided also are compositions (e.g., pharmaceutical compositions) for use in the methods of the disclosure. In embodiments, the composition is a pharmaceutical composition for use in treating a disease or disorder (e.g., a neoplasia). In some instances, a composition of the disclosure is used in a diagnostic method (e.g., to detect a marker associated with a disease). In some cases, a composition of the disclosure is used in one of the screening methods provided herein. In an embodiment, the compositions contain a cell, polynucleotide, vector, or polypeptide (e.g., a VHH antibody) provided herein. In some cases, the composition contains CAR-T cells or CAR NK cells, as described herein and an acceptable carrier, excipient, or diluent.

The agents of the disclosure (e.g., polynucleotides, polypeptides, vectors, and/or cells) may be contained in any appropriate amount in any suitable carrier substance and is/are present in some cases in an amount of 0.01-95% by weight of the total weight of the composition. A pharmaceutical composition may be provided in a form that is suitable for a parenteral (e.g., subcutaneous, intravenous, intramuscular, or intraperitoneal) administration route, such that the agent, such as a vector or cell described herein, is systemically delivered.

The compositions of the present disclosure can be prepared in accordance with known techniques. See, e.g., Remington, The Science And Practice of Pharmacy (21st ed. 2005). In some embodiments, an agent of the disclosure is present in a reconstitutable dry composition (e.g., a lyophilized composition or powder). In embodiments, an agent is admixed with a suitable carrier prior to administration or storage, and in some embodiments, the composition further comprises an acceptable carrier (e.g., a pharmaceutically acceptable carrier). Suitable pharmaceutically acceptable carriers generally comprise inert substances that aid in administering the pharmaceutical composition to a subject, aid in processing the pharmaceutical compositions into deliverable preparations, or aid in storing the pharmaceutical composition prior to administration. Carriers can include agents that can stabilize, optimize or otherwise alter the form, consistency, viscosity, pH, pharmacokinetics, or solubility of a composition. Such agents include buffering agents, wetting agents, emulsifying agents, diluents, encapsulating agents, and skin penetration enhancers. For example, carriers can include, but are not limited to, saline, buffered saline, dextrose, arginine, sucrose, water, glycerol, ethanol, sorbitol, dextran, sodium carboxymethyl cellulose, and combinations thereof.

Some nonlimiting examples of materials which can serve as carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Rin'er's solution; (19) ethyl alcohol; (20) pH buffered solutions; (21) polyesters, polycarbonates and/or polyanhydrides; (22) bulking agents, such as polypeptides and amino acids (23) serum alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation.

Compositions of the disclosure can contain one or more pH buffering compounds to maintain the pH of the formulation at a predetermined level that reflects physiological pH, such as in the range of about 5.0 to about 8.0. The pH buffering compound used in the aqueous liquid formulation can be an amino acid or mixture of amino acids, such as histidine or a mixture of amino acids such as histidine and glycine. Alternatively, the pH buffering compound is an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Compositions can also contain one or more osmotic modulating agents, i.e., a compound that modulates the osmotic properties (e.g., tonicity, osmolality, and/or osmotic pressure) of the formulation to a level that is acceptable, for example, to the blood stream and blood cells of recipient subjects. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to: salts, such as sodium chloride and sodium acetate; sugars, such as sucro69uspensiose, and mannitol; amino acids, such as glycine; and mixtures of one or more of these agents and/or types of agents. The osmotic modulating agent(s) may be present in any concentration sufficient to modulate the osmotic properties of the formulation.

The skilled artisan can readily determine the number of cells and amount of optional additives, vehicles, and/or carriers in compositions and to be administered in methods of the disclosure. Of course, for any composition to be administered to an animal or human, and for any particular method of administration, it is advantageous to determine therefore: toxicity, such as by determining the lethal dose (LD) and LD50 in a suitable animal model (e.g., a rodent such as a mouse); and, the dosage of the composition(s), concentration of components therein, and the timing of administering the composition(s), which elicit a suitable response. Such determinations do not require undue experimentation from the knowledge of the skilled artisan, this disclosure and the documents cited herein, and the time for sequential administrations can be ascertained without undue experimentation.

In some embodiments, the composition is formulated for delivery to a subject. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration. The pharmaceutical composition may be administered systemically.

The composition may be in the form of a solutio70uspensionsion, an emulsion, an infusion device, or a delivery device for implantation, or it may be presented as a dry powder to be reconstituted with water or another suitable vehicle before use. Apart from the agent (e.g., CAR-T cells, CAR NK cells, VHH antibodies, polynucleotides, or polypeptides provided herein), the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

In some embodiments, the composition is formulated for intravenous delivery. The compositions according to the described embodiments may be in a form suitable for sterile injection. To prepare such a composition, the suitable therapeutic(s) are dissolved or suspended in a parenterally acceptable liquid vehicle. Acceptable vehicles and solvents that may be employed include water, water adjusted to a suitable pH by addition of an appropriate amount of hydrochloric acid, sodium hydroxide or a suitable buffer, 1,3-butanediol, Rin'er's solution, isotonic sodium chloride solution and dextrose solution. The aqueous formulation may also contain one or more preservatives (e.g., methyl, ethyl, or n-propyl p-hydroxybenzoate). In cases where one of the agents is only sparingly or slightly soluble in water, a dissolution enhancing or solubilizing agent can be added, or the solvent may include 10-60% w/w of propylene glycol or the like.

Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, domesticated animals, pets, and commercially relevant mammals such as cattle, pigs, horses, sheep, cats, dogs, mice, and/or rats; and/or birds, including commercially relevant birds such as chickens, ducks, geese, and/or turkeys.

Except insofar as any conventional excipient medium is incompatible with a substance or its derivatives, such as by producing any undesirable biological effect or otherwise interacting in a deleterious manner with any other component(s) of the composition, its use is contemplated to be within the scope of this disclosure.

In some embodiments, compositions in accordance with the present disclosure can be used for treatment of any of a variety of diseases, disorders, and/or conditions.

### Treatments

The compositions, polynucleotides, cells (e.g., chimeric antigen receptor T or NK cells), and/or polypeptides (e.g., polypeptides containing a VHH antibody identified by the methods provided herein) provided herein can be used for treating a subject for a disease or disorder, such as a neoplasia (e.g., a lung cancer or an ovarian cancer). Generally, the methods provided herein include administering a therapeutically effective amount of an agent as provided herein, to a subject who is in need of, or who has been determined to be in need of, such treatment.

The methods provided herein include selecting a subject for and/or administering to a subject having or having a propensity to develop a neoplasia a treatment that includes a therapeutically effective amount of an immunotherapeutic agent, such as a CAR-T cell or CAR NK cell of the disclosure.

In some instances, the methods provided herein involve administering to a subject in need thereof a VHH domain provided herein that has been conjugated to an agent (e.g., a cytotoxic agent). Antibody-drug conjugates are described, e.g., in Zolog, et al. “Antibody-drug conjugates,” Nature Reviews Drug Discovery, 12:259-260 (2013), the disclosure of which is incorporated herein by reference in its entirety for all purposes.

An effective amount of an agent can be administered in one or more administrations, applications or dosages. A therapeutically effective amount of a therapeutic compound or agent (i.e., an effective dosage) depends on the therapeutic compounds or agents selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic agents provided herein can include a single treatment or a series of treatments.

Dosage, toxicity and therapeutic efficacy of the therapeutic agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Agents which exhibit high therapeutic indices may be used. While agents that exhibit toxic side effects may be used, care should be taken to design a delivery system that targets such agents to the site of affected tissue in order to minimize potential damage to uninfected cells and, thereby, reduce side effects.

The data obtained from cell culture assays and animal studies can be used in formulating a range of dosage for use in humans. The dosage of such agents lies in various embodiments within a range of circulating concentrations that include the ED50 with little or no toxicity. The dosage may vary within this range depending upon the dosage form employed and the route of administration utilized. For any agent used in the method of the disclosure, the therapeutically effective dose can be estimated initially from cell culture assays. A dose may be formulated in animal models to achieve a circulating plasma concentration range that includes the IC₅₀ (i.e., the concentration of the test agent which achieves a half-maximal inhibition of symptoms) as determined in cell culture. Such information can be used to determine useful doses more accurately in humans. Levels in plasma may be measured, for example, by high performance liquid chromatography.

Dosages and desired drug concentration of pharmaceutical compositions of the present disclosure may vary depending on the particular use envisioned. The determination of the appropriate dosage or route of administration (e.g., oral administration, intravenous administration as a bolus or by continuous infusion over a period of time, by intramuscular, intraperitoneal, intracerobrospinal, intracranial, intraspinal, subcutaneous, intraarticular, intrasynovial, intrathecal, topical, or inhalation routes) is well within the skill of an ordinary artisan. Animal experiments provide reliable guidance for the determination of effective doses for human therapy. Interspecies scaling of effective doses can be performed following the principles described in Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

For in vivo administration of any of the agents of the present disclosure, normal dosage amounts may vary from about 10 ng/kg up to about 100 mg/kg of an individ'al's and/or subj'ct's body weight or more per day, depending upon the route of administration. In some embodiments, the dose amount is about 1 mg/kg/day to 10 mg/kg/day. In some embodiments, the dose amount of a CAR-T cell is about, at least about, and/or no more than about 1e5 cells, 1e6 cells, 1e7 cells, 1e8 cells, 1e9 cells, 1e10 cells, 1e11 cells, 1e12 cells, 1e13 cells, 1e14 cells, 1e15 cells, or 1e16 cells. For repeated administrations over several days or longer, depending on the severity of the disease, disorder, or condition to be treated, the treatment is sustained until a desired suppression of symptoms is achieved.

An effective amount of an agent of the instant disclosure may vary, e.g., from about 0.001 mg/kg to about 1000 mg/kg or more in one or more dose administrations for one or several days (depending on the mode of administration). In certain embodiments, the effective amount per dose varies from about 0.001 mg/kg to about 1000 mg/kg, from about 0.01 mg/kg to about 750 mg/kg, from about 0.1 mg/kg to about 500 mg/kg, from about 1.0 mg/kg to about 250 mg/kg, and from about 10.0 mg/kg to about 150 mg/kg.

An exemplary dosing regimen may include administering an initial dose of an agent of the disclosure of about 200 μg/kg, followed by a weekly maintenance dose of about 100 μg/kg every other week. Other dosage regimens may be useful, depending on the pattern of pharmacokinetic decay that the physician wishes to achieve. For example, dosing an individual from one to twenty-one times a week is contemplated herein. In certain embodiments, dosing ranging from about 3 μg/kg to about 2 mg/kg (such as about 3 μg/kg, about 10 μg/kg, about 30 μg/kg. about 100 μg/kg, about 300 μg/kg, about 1 mg/kg. or about 2 mg/kg) may be used. In certain embodiments, dosing frequency is three times per day, twice per day, once per day. once every other day. once weekly, once every two weeks, once every four weeks, once every five weeks, once every six weeks, once every seven weeks, once every eight weeks, once every nine weeks, once every ten weeks, or once monthly, once every two months, once every three months, or longer. Progress of the therapy is easily monitored by conventional techniques and assays. The dosing regimen, including the agent(s) administered, can vary over time independently of the dose used.

Methods for characterizing the efficacy of a treatment for a neoplasia are well known in the art (e.g., computerized tomography (CT) scan, bone scan, magnetic resonance imaging (MRI), position emission tomography (PET) scan, ultrasound X-ray, biopsy, etc.).

### Substantially Identical Amino Acid and Nucleotide Sequences for VHHs

There is a large body of information in the literature supporting the fact that closely related antibody (Ab) sequences are capable of performing the same binding and therapeutic functions such that this is now generally accepted by those with ordinary skill in the art of immunological sciences. The creation of Abs with small numbers of amino acid sequence variations occurs naturally within mammals and some other animal species during the process of ‘affinity maturation’ in which Ab-producing cells that bind a newly encountered antigen (Ag) are expanded, and their progeny cells contain random mutations within portions of the Ab coding DNA that results in new, related Ab sequences. The cells expressing Abs that have gained improved binding properties for the new Ag are then selected and expanded, thereby increasing the amount of the improved antibody in the animal. This process continues through multiple generations of mutation and selection until Abs with greatly improved antigen binding properties result. The process of Ab affinity maturation demonstrates that related, yet not identical, Ab amino acid sequences can possess similar target binding properties and perform similar therapeutic functions in vivo.

The present disclosure provides anti-mesothelin VHH antibodies having related sequences that are capable of binding mesothelin. The Abs described herein are heavy-chain only, single domain VHH antibodies, which are generated in camelid alpacas, which have been reported to be convenient sources of camelid VHH antibodies (See, e.g., Maass, D. R. et al., 2007, J. Immunol. Methods, 324:13-25). Briefly, an animal capable of producing VHH antibodies in response to an antigen are immunized with a selected mesothelin antigen (mesothelin Ag) one or multiple times to permit the animal to undergo affinity maturation of the anti-mesothelin VHHs that are produced. Anti-mesothelin VHHs are then isolated and the encoding DNA selected for expression of soluble VHHs that bind mesothelin Ag and have potential therapeutic or diagnostic properties. During this process, many examples of closely related anti-mesothelin binding VHHs are isolated, which are distinctive, and which are presumably intermediates that result from the affinity maturation process which occurs during anti-mesothelin VHH production in alpaca lymphocytes. These related anti-mesothelin VHHs are screened for binding to mesothelin Ag, and the most promising members of homology groups of mesothelin-binding VHHs are identified and become lead candidates for further development.

Similar to all mammalian antibodies, VHHs consist of four, well-conserved ‘framework’ regions (FRs) which are important in forming the antibody structure. Between the FRs (FR1, FR2, FR3 and FR4) are three much less well-conserved CDRs or hypervariable regions (CDR1, CDR2 and CDR3) which principally interact with and bind to antigenic determinants or epitopes on antigens (Ags), such as mesothelin. The CDR sequences vary widely so as to interact and bind to epitopes of Ags. The third CDR, CDR3, is generally the longest in sequence and is most diverse of the CDRs within VHHs, both in size and sequence. By way of nonlimiting example, CDR3 in VHHs can range in size from about 5 to about 30 amino acid residues. Without intending to be bound by theory, VHHs and CDR3 regions that bind to the same mesothelin target Ag are considered to have resulted from affinity maturation of a common precursor VHH within the animal and are classified as a ‘homology group.’ Individual VHHs within a homology group are classified by their binding to the target Ag, and the members of the VHH homology group are able to ‘compete’ with each other for binding to the Ag, thus demonstrating that they bind to the same region on the target Ag. In VHH molecules, the CDRs (CDR1, CDR2 and CD3) play a role in the ability of a VHH to bind to the target Ag, e.g., mesothelin, in conjunction with CDR1 and CDR2.

Since the FRs maintain the structure of a VHH and the positioning of the CDRs for binding to the target Ag, the FRs of VHHs typically do not vary extensively in sequence (FIG. 1). However, some VHH FR amino acid sequence variation is permissible, particularly in cases in which an amino acid substitution involves the replacement or substitution of one amino acid with another amino acid having similar properties (e.g., similarity in being charged or uncharged), i.e., a conservative substitution. Such conservative changes in FRs can often be found naturally within VHHs that have undergone affinity maturation in an animal. Similar to the case with FRs, VHH CDRs also typically do not vary extensively in amino acid sequence or type so as not to compromise their ability to specifically bind to Ag. As would be appreciated by one skilled in the art, an estimation of the extent of amino acid sequence variation that can be tolerated within VHHs without compromising their Ag binding ability can be made by observing the variation that occurs naturally within affinity-matured homology groups of VHHs isolated from the same types of animals and which bind to the same Ag.

In an embodiment, sequence variation is particularly acceptable in the CDR regions, e.g., CDR1, CDR2, and/or CDR3, while the feature of VHH binding to antigen mesothelin is maintained. In an embodiment, amino acid sequence variation results from conservative amino acid substitutions in a VHH sequence. In an embodiment, the conservative amino acid substitutions are in one or more CDR sequences of the VHH polypeptide. In an embodiment, the conservative amino acid substitutions are in one or more FR sequences of the VHH polypeptide. In an embodiment, the conservative amino acid substitutions are in one or more CDR sequences and in one or more FR sequences of the VHH polypeptide.

An example evidencing that VHH sequence variation is acceptable within related VHHs having the same Ag binding characteristics is described in Tremblay et al., 2013, Infect Immun 81:4592-4603. In this report, 11 VHH sequences comprise a large homology group with closely related CDR3 sequences, and the unusual property of cross-specific binding to two different Shiga toxins, Stx1 and Stx2. Two of the more distantly related VHH members of this homology group are characterized as having common Ag binding characteristics. These two related VHHs were found to have 32 amino acid changes in the total VHH sequence of 120 or 121 residues. Thus, a 26% variation in amino acid sequence did not adversely affect the common Ag binding properties of the VHH proteins.

### Kits

The disclosure also provides kits for use in the methods of the disclosure. Kits of the instant disclosure may include one or more containers comprising an agent provided herein, such as a polypeptide (e.g., a VHH antibody) or a polynucleotide encoding the same or a cell (e.g., a CAR-T cell). In some embodiments, the kits further include instructions for use in accordance with the methods of this disclosure. In some embodiments, these instructions comprise a description of use of the agent to treat a disease or identify VHH antibodies that bind a target antigen. The kit may further comprise a description of how to analyze and/or interpret data.

Instructions supplied in the kits of the instant disclosure are typically written instructions on a label or package insert (e.g., a paper sheet included in the kit), but machine-readable instructions (e.g., instructions carried on a magnetic or optical storage disk) are also acceptable. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. Kits may optionally provide additional components such as buffers and interpretive information. Normally, the kit comprises a container and a label or package insert(s) on or associated with the container.

The practice of the various aspects and embodiments of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the disclosure, and, as such, may be considered in making and practicing the various aspects and embodiments of the disclosure. Particularly useful techniques for specific embodiments will be discussed in the sections that follow.

The following examples are put forth so as to provide those of ordinary skill in the art with a complete disclosure and description of how to make and use the assay, screening, and therapeutic methods of the disclosure, and are not intended to limit the scope of what the inventors regard as their invention.

### EXAMPLES

### Example 1: Discovery of Anti-Mesothelin VHH Antibodies

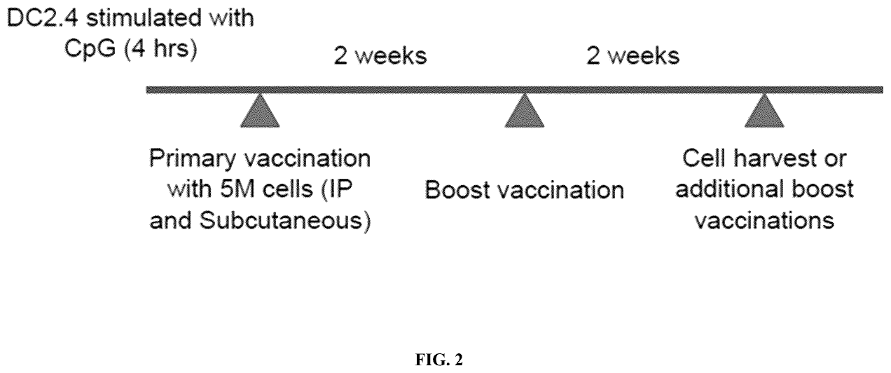

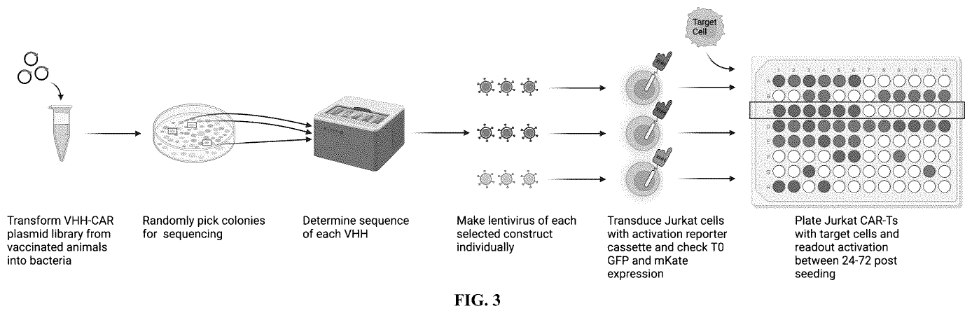

Experiments were undertaken to develop VHH antibodies capable of binding mesothelin. To develop the VHH antibodies, mice (i.e., nanomice) capable of producing VHH antibodies (i.e., nanobodies) in response to an antigen were vaccinated with a live vaccine containing antigen presenting cells expressing mesothelin (FIG. 2). The antigen presenting cells were DC2.4 cells transduced with expression constructs encoding mesothelin. The transduced DC2.4 cells surface-presented mesothelin antigens. Evaluation of serum samples from the vaccinated nanomice confirmed that the mice produced anti-mesothelin VHH antibodies.

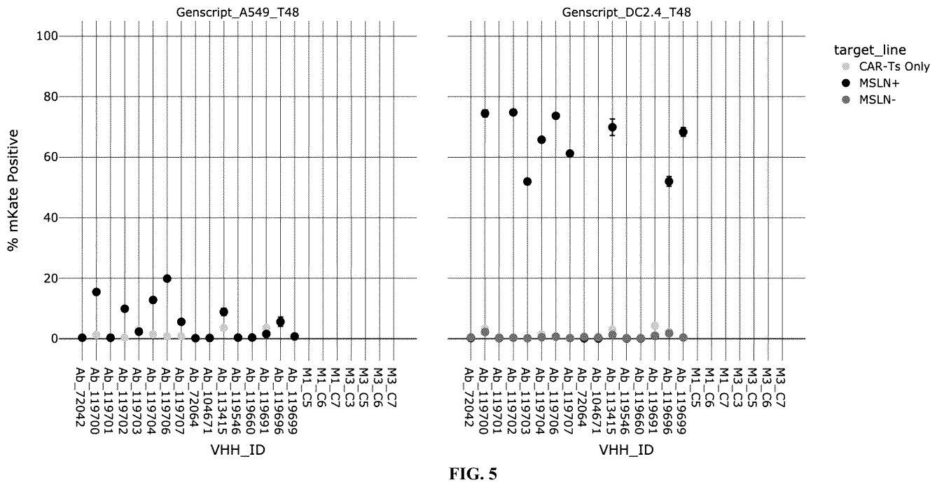

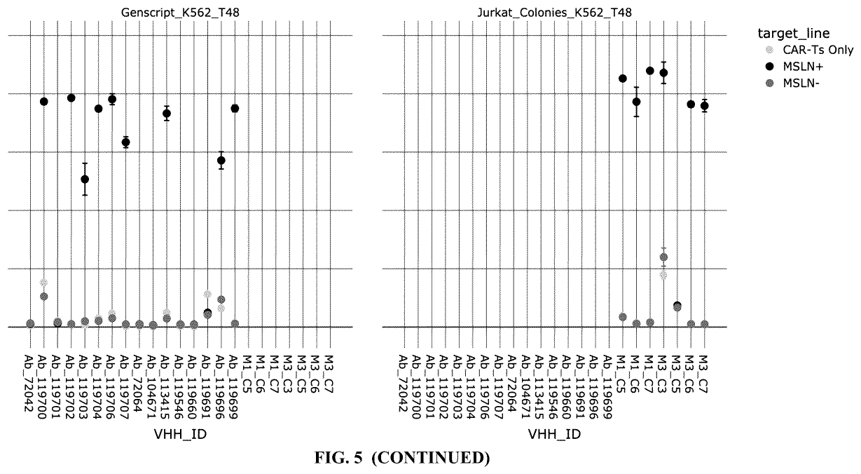

Having established that the vaccinated nanomice produced anti-mesothelin nanobodies, experiments were undertaken (e.g., as shown in FIG. 3) to clone and characterize anti-mesothelin VHH antibodies produced by the mice. First, cDNA was prepared using nanobody-encoding mRNA from the nanomice and the encoded nanobodies were cloned in-frame into an expression vector encoding a chimeric antigen receptor (CAR) such that the nanobodies would function as the antigen-binding domain of the encoded CAR. The chimeric antigen receptors contained from N-terminus to C-terminus a CD28 signal peptide, a cloned VHH domain as an antigen-binding domain (ABD), a CD8 hinge domain, a CD28 transmembrane domain, a CD28 cytoplasmic domain, and a CD3ζ domain. The expression construct further encoded an enhanced GFP (eGFP) protein linked to the C-terminus of the CAR by a P2A self-cleaving peptide. The expression vectors were cloned into bacteria and colonies containing the expression vectors were randomly selected and the VHH domains of the expression vectors corresponding to each colony were sequenced. Next, following sequencing, the five VHH domains M3_C7, Ab_119706, Ab_119699, Ab_119704, and Ab_119707 (sequences provided in Tables 1-3B) were selected for further characterization using flow cytometry. Lentiviral vectors containing polynucleotides encoding the selected sequenced chimeric antigen receptors (CAR) were prepared and transduced into Jurkat cells containing an activation reporter cassette that expressed the fluorescent protein mKate when the cells expressing the CARs were activated by the CARs binding mesothelin. The Jurkat cells expressing the CARs were co-cultured with target cells surface-presenting mesothelin (MSLN+) for about 48 hours and expression of GFP (to measure CAR expression) and mKate (to measure cell activation) was measured. As controls, the CAR-T Jurkat cells were also cultured in the presence of cells that did not express mesothelin (MSLN-) or were cultured in the absence of any other cells (i.e., CAR-Ts only). All five of the CAR-expressing Jurkat cells were activated when co-cultured with the target cells (e.g., FIG. 5).

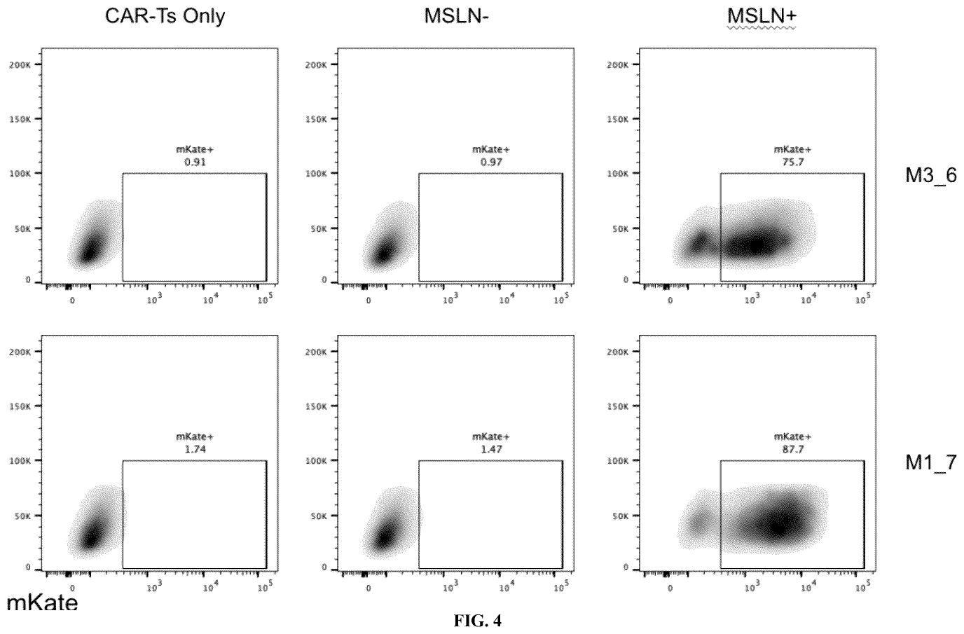

In addition to the characterization of the five anti-mesothelin nanobodies characterized as described above, 14 VHH antibodies and two negative controls (AB 72064, which was a non-VHH control, and ABE_72042, which was a nonsense sequence that did not encode any functional VHH) were selected for synthesis into pSLCAR vectors (see below sequence), which were then used to prepare Jurkat reporter CAR-T cells as described above. These cells were then co-cultured in the presence of MSLN+target cells and activation was measured using flow cytometry (FIGS. 4 and 5). As controls, the CAR-T Jurkat cells were also cultured in the presence of cells that did not express mesothelin (MSLN-) or were cultured in the absence of any other cells (i.e., CAR-Ts only). Many of these additional VHHs were also associated with activation of Jurkat cells expressing chimeric antigen receptors (CARs) containing the VHHs as antigen binding domains. The analysis of these 14 VHHs led to the discovery of the following VHH antibodies: M1_C8, M3_C3, Ab_119700, Ab_119701, M3_C6, Ab_119696, Ab_113415, and Ab_119702 (see sequences provided in sequences provided in Tables 1-3B). Sequences for further polypeptides evaluated are provided in Table 7.

### Example 2: In Vivo Validation of Anti-Mesothelin (MSLN) VHH Antibodies

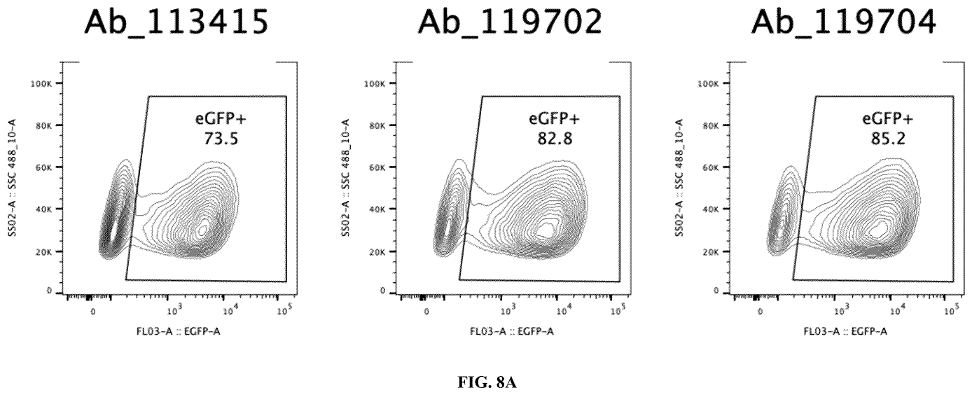

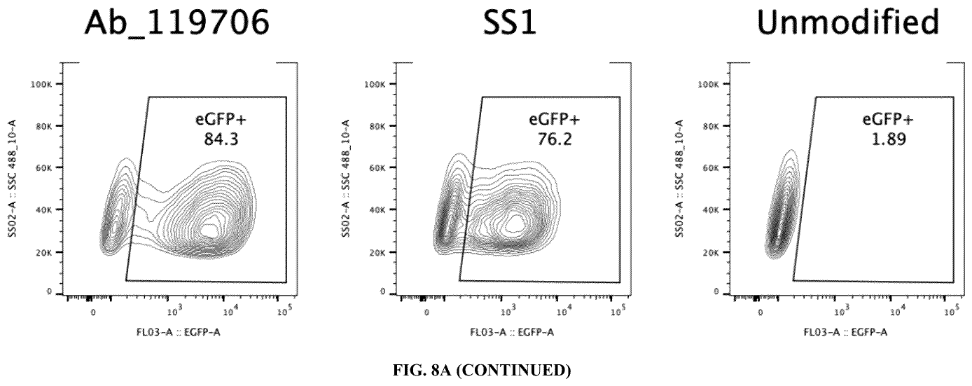

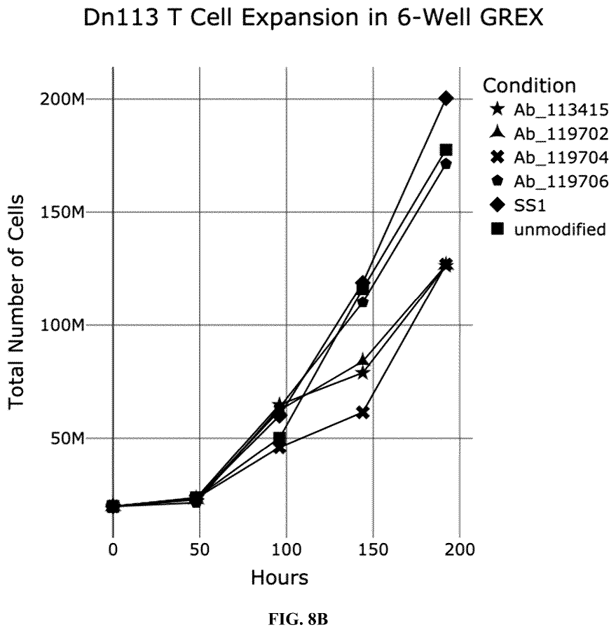

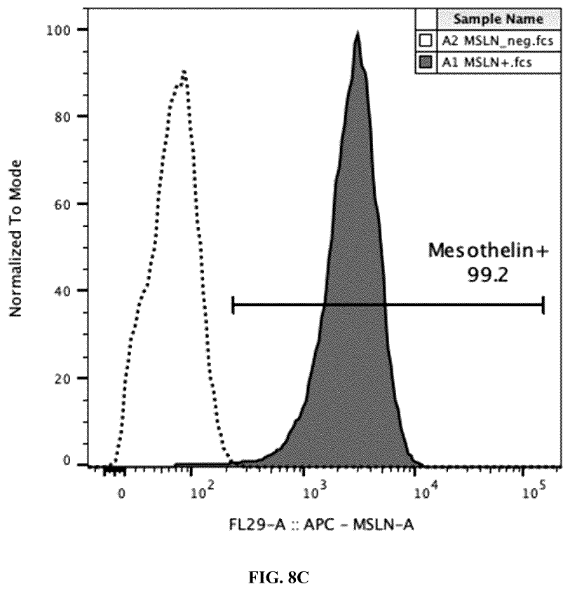

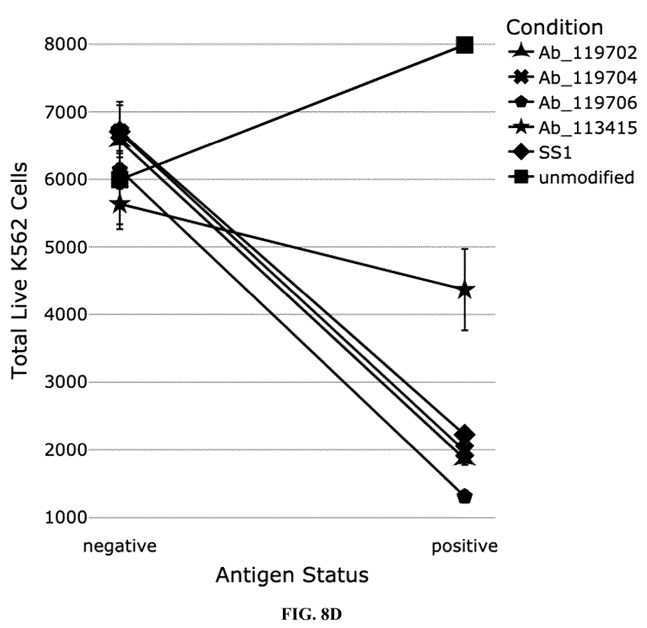

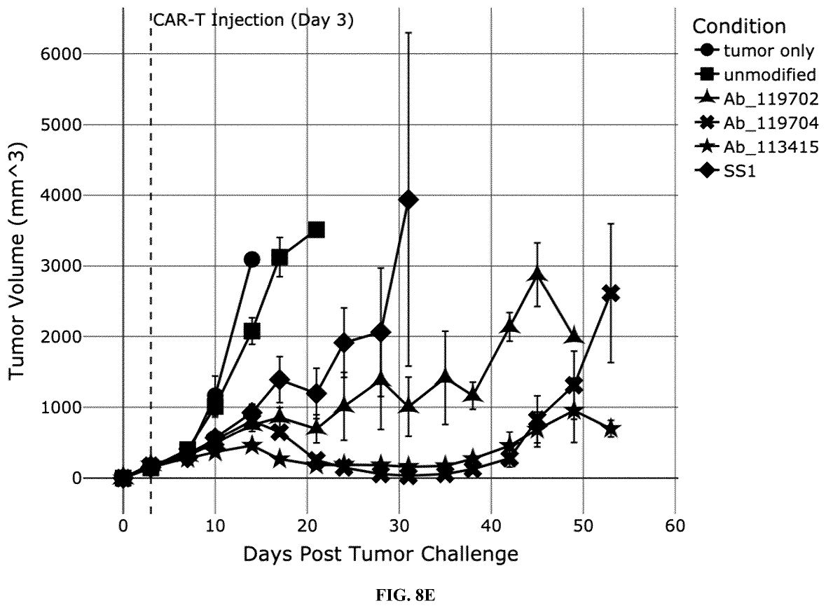

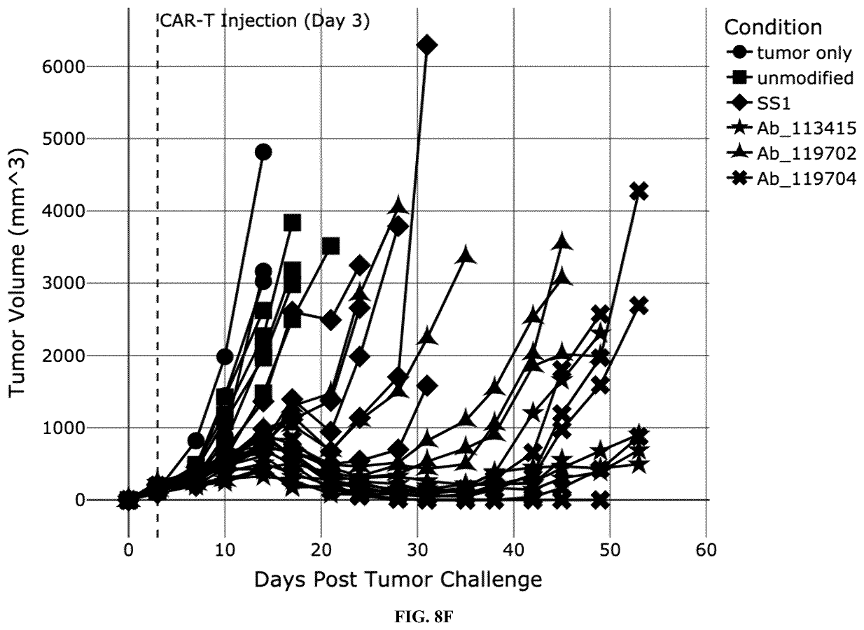

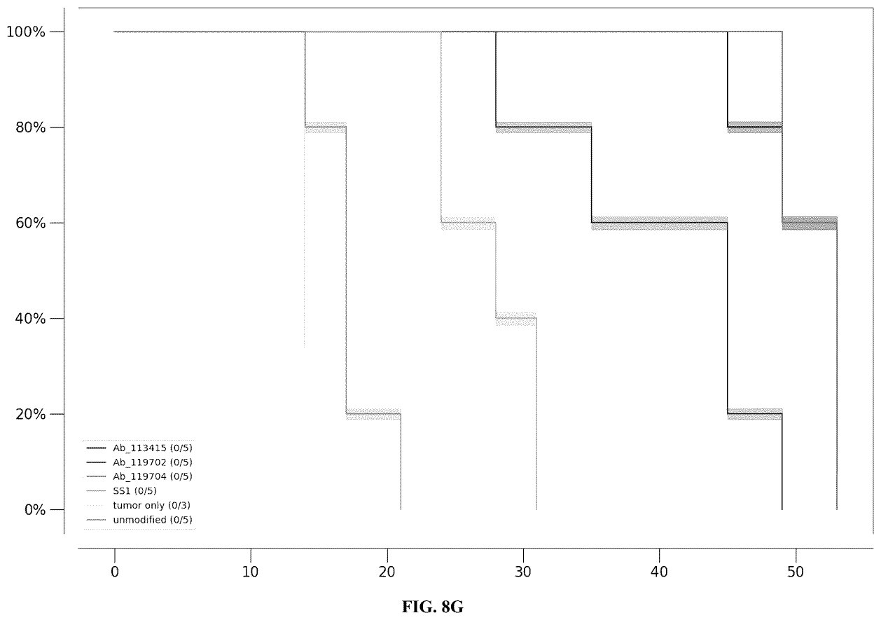

Experiments were undertaken to validate the ability of the anti-MSLN VHH antibodies to function as antigen-binding domains of chimeric antigen receptors expressed by T cells to enable the chimeric antigen receptor-expressing T cells (i.e., CAR-T cells) to control tumors in vivo. 1e9 peripheral blood mononuclear cells (PBMCs) were obtained from StemCell Technologies and CD3 positive T cells were isolated using negative bead-based selection. The isolated T cells were immediately activated using CD3/28 activation Dynabeads (Gibco, 11131D) 1:1. After 24 hours activation, lentivirus vectors were used to introduce expression constructs encoding the chimeric antigen receptor polypeptides to the T cells. The chimeric antigen receptor polypeptides contained one of the following antibodies as an antigen binding domain: Ab_113415, Ab_119702, Ab_119704, Ab_119706, and SS1. The CARs containing an SS1 antigen-binding domain (“SS1 CARs”) were used as a positive control (see, e.g., Castelletti, et al. Biomark Res 9, 11 (2021) doi.org/10.1186/s40364-021-00264-1, the disclosure of which is hereby incorporated by reference in its entirety for all purposes). Amino acid and nucleotide sequences for the chimeric antigen receptors containing the VHH antibodies as antigen binding domains are provided at Table 9. Lentivirus and activation beads were removed at 48 hours post-activation. The resulting CAR-T cells (“the effector cells”) were allowed to expand for 10 days (FIGS. 8A and 8B). During CAR-T expansion, K562_pLX311-MSLN target cells (i.e., K562 cells modified to surface-express mesothelin) were simultaneously expanded for use to be administered subcutaneously (SC) to mice. The target cells were checked to ensure purity of MSLN expression (>99%) prior to injection (FIG. 8C). 1.5e6 K562_pLX311-MSLN cells were resuspended in 50% HBSS, 50% growth factor reduced Matrigel (VWR International LLC, 47743-722) and injected SC into the right flank of NSG mice. An in vitro cytotoxicity assay was also performed using an effector (CAR-T cells) to target (K562_pLX311-MSLN cells) cell ratio (E:T) of 1:3 (FIG. 8D) confirming the ability of the CAR-T cells to kill the target cells. 3 days following the SC injection (Day 3) of the target cells, the mice were tail vein injected with 5e6 of the CAR-T cells. Tumor growth was measured every 3-4 days for the duration of the experiment to assess CAR-T efficacy (FIGS. 8E and 8F). As expected, the tumors rapidly grew in mice that were not treated or that received unmodified T cells (i.e., T cells that did not express a CAR). Compared to T cells expressing the SS1 CARs, the clinical benchmark, the CAR-T cells expressing CARs containing VHH antibodies as their antigen-binding domains demonstrated superior tumor control, particularly in the case of the VHH antibodies Ab_113415 and Ab_119704. Survival was significantly extended for mice administered CAR-T cells expressing CARs containing VHH antibodies as their antigen-binding domains, as compared to mice administered CAR-T cells expressing the SS1 CARs (FIG. 8G).

### Example 3: Characterization of the Ability of Anti-Mesothelin (MSLN) VHH Antibodies to Bind Mesothelin

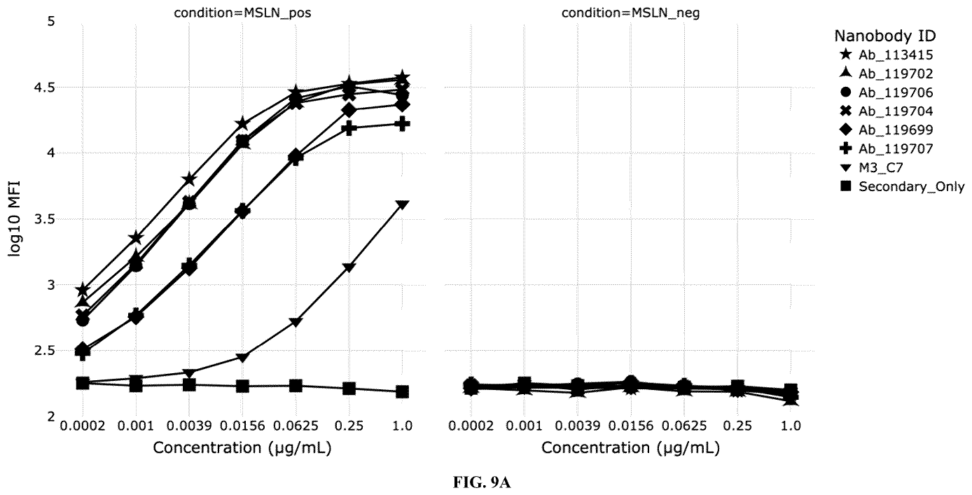

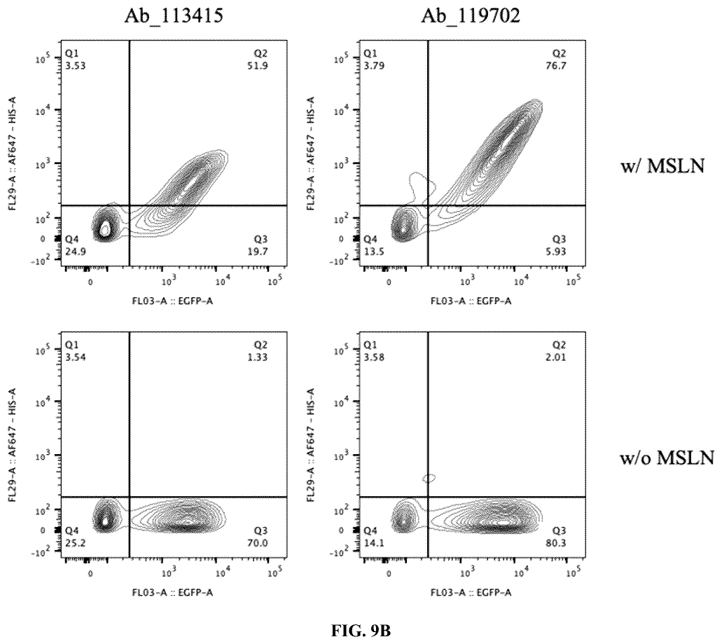

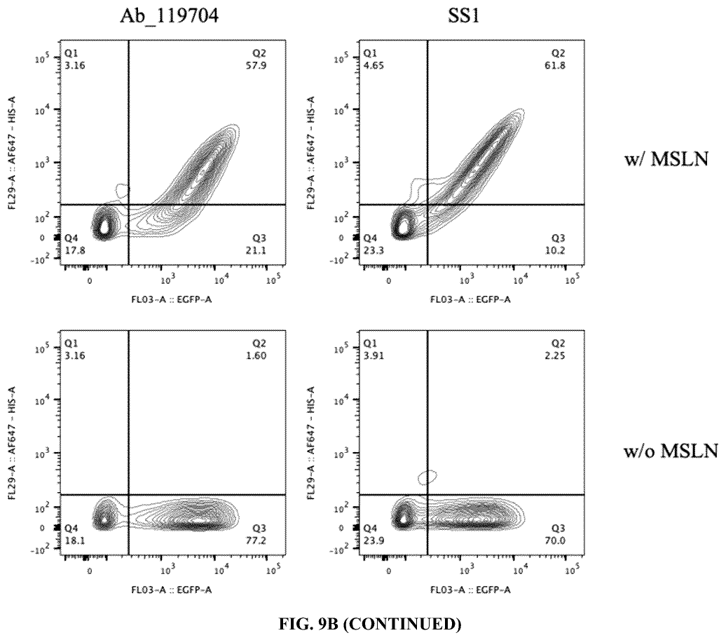

Experiments were undertaken to characterize the ability of the anti-MSLN VHH antibodies to bind MSLN. 6×His tagged (SEQ ID NO: 93) VHH antibodies were produced by Genscript in a mammalian cell line and purified. The purified VHH antibodies were tested using a flow cytometry-based assay to see how well they bound mesothelin expressed on cells (FIG. 9A). K562_pLX311-MSLN (i.e., K562 cells modified to surface-express mesothelin) and unmodified K562 cells that did not surface-express mesothelin were immunostained using the purified nanobodies at concentrations ranging from <0.001p g/mL to 1.0 μg/mL (FIG. 9A). Also, biolayer interferometry (BLI) was used to accurately assess the binding affinity (K_{d}) of the anti-MSLN VHH antibodies (Table 5). BLI was conducted with soluble MSLN (Glu2-6-Gly580, Acro Bio Systems, MSN-H82E9-25 ug). Of the anti-MSLN VHH antibodies tested, at least 5 bound MSLN with K_{d}<12 nM. In addition to determining the K_{d} of the anti-MSLN VHH antibodies, the ability of the CAR-T cells used in the in vivo experiments described above in Example 2 to bind soluble MSLN was measured (FIG. 9B) by exposing the CAR-T cells to media containing soluble MSLN (Glu2-6-Gly580, Acro Bio Systems, MSN-H82E9-25 ug) at 1.0 μg/mL for 30 minutes at 4° C. After exposure to the media containing the soluble MSLN, an anti-His secondary antibody was used to detect CAR-T cells binding the MSLN. T cells expressing CARs containing the anti-MSLN VHH antibodies or SS1 as antigen binding domains bound the soluble MSLN.

TABLE 5
K_{d} values for binding to MSLN for the indicated VHH antibodies

| Nanobody ID | K_{d} (nM) |
|---|---|
| Ab_119699 | Not Determined |
| Ab_113415 | 3.689 |
| Ab_119704 | 4.493 |
| Ab_119702 | 5.320 |
| Ab_119707 | 5.953 |
| Ab_119706 | 11.510 |

### Example 4: Binding of Anti-Mesothelin (MSLN) VHH Antibodies to Endogenously Expressed MSLN

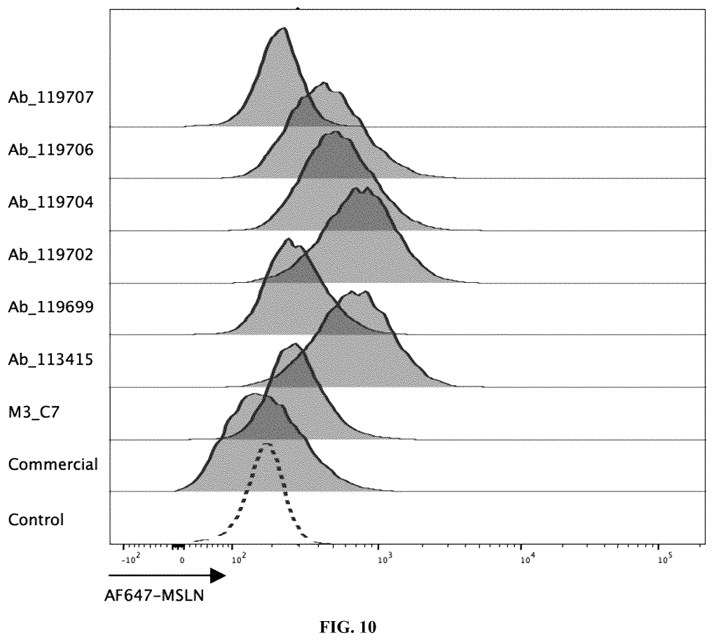

Experiments were undertaken demonstrate that the anti-MSLN VHH antibodies were capable of binding endogenously expressed MSLN. The AsPC-1 (ATCC, CRL-1682) human pancreatic cell line was chosen in part based on predicted high MSLN expression from RNAseq data. AsPC-1 cells were immunostained by first contacting the cells with the anti-MSLN VHH antibodies produced as described in Example 4 followed by being contacted with anti-His antibodies conjugated to an AF647 fluorescent dye. Additionally, as a control, AsPC-1 cells were labeled with a commercially available anti-MSLN antibody (R&D Systems, FAB32652A). Several of the anti-MSLN VHH antibodies were capable of binding endogenously expressed MSLN (FIG. 10). Interestingly, the commercially available antibody, which was intended to act as positive control, was not effective in immunostaining the cells.

### Example 5: Clustering of Anti-Mesothelin (MSLN) VHH Antibody Sequences

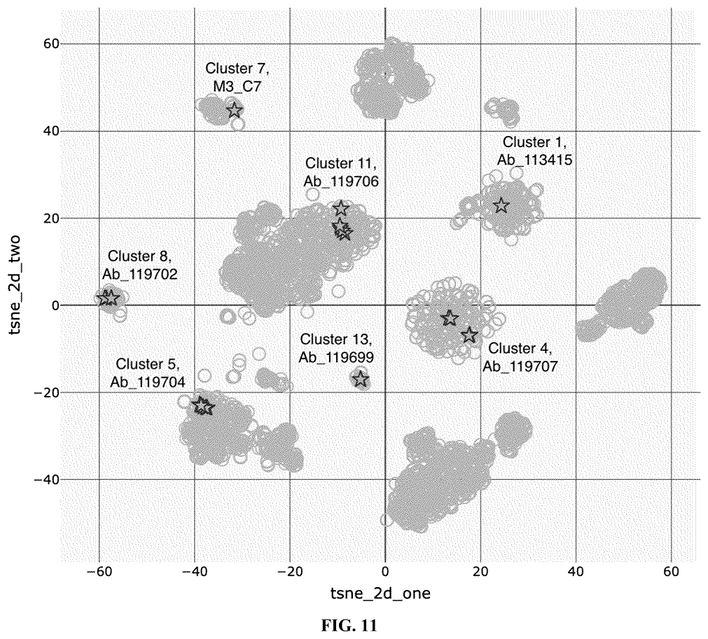

To identify maximally diverse anti-MSLN VHH antibody sequences, full-length anti-MSLN VHH antibody amino acid sequences were clustered and projected into t-SNE space (FIG. 11). Clustering was performed by analyzing the amino acid relationships using the Cluster K-Spaced Amino Acid algorithm to determine similarity/dissimilarity between each sequence. The sequence clonotype abundance threshold was set to >=5. The resulting relationship feature matrix was dimensionally reduced to 2D space by t-SNE and then further cluster architecture was established by using K-means nearest neighbor. In addition, sequences from the anti-MSLN VHH antibodies M3 C7, Ab_119702, Ab_119706, Ab_113415, Ab_119704, Ab_119699, and Ab_119707 were mapped onto the data (see stars in FIG. 11). The plot revealed seven distinct clusters to which each anti-MSLN VHH antibody belonged. The sequences for all polypeptides falling within a cluster containing the M3_C7, Ab 119702, Ab_119706, Ab 113415, Ab 119704, Ab 119699, or Ab_119707 are listed in Tables 8A-8G below. The sequences within each cluster were highly similar and may represent additional MSLN binders.

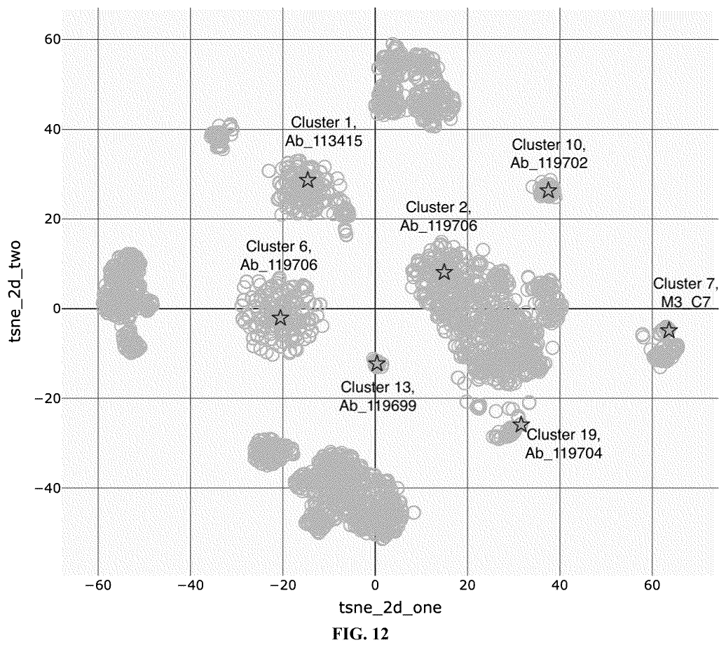

A further clustering analysis of the amino acid sequences was completed according to a method similar to that described above but using sequences more stringently filtered to assist in the improvement of the feature matrix used to prepare the clusters. Sequences from the anti-MSLN VHH antibodies M3_C7, Ab 119702, Ab 119706, Ab 113415, Ab_119704, Ab_119699, and Ab_119707 were mapped onto the data (see stars in FIG. 12). The sequences for all polypeptides falling within each cluster are listed in Tables 10A-10M below, and consensus sequences for complementarity determining regions corresponding to each cluster are provided in Table 11.

### Sequences

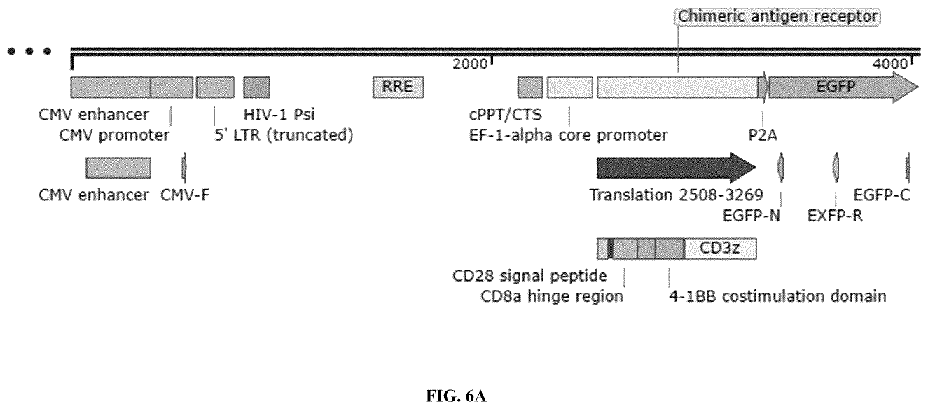

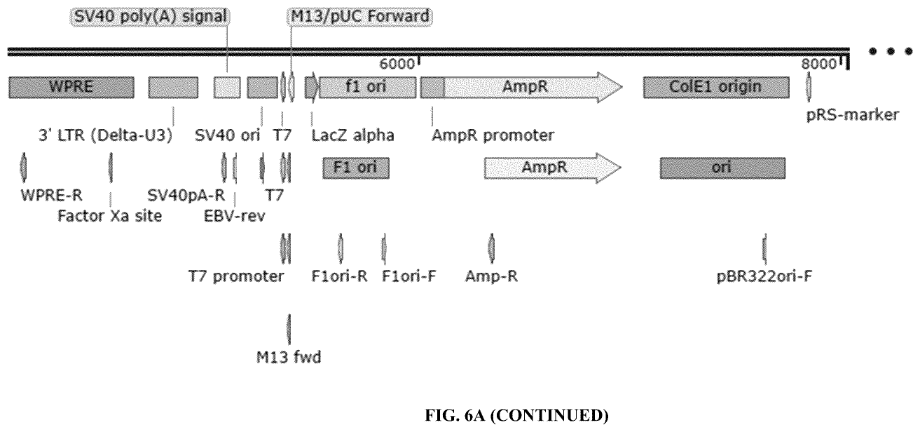

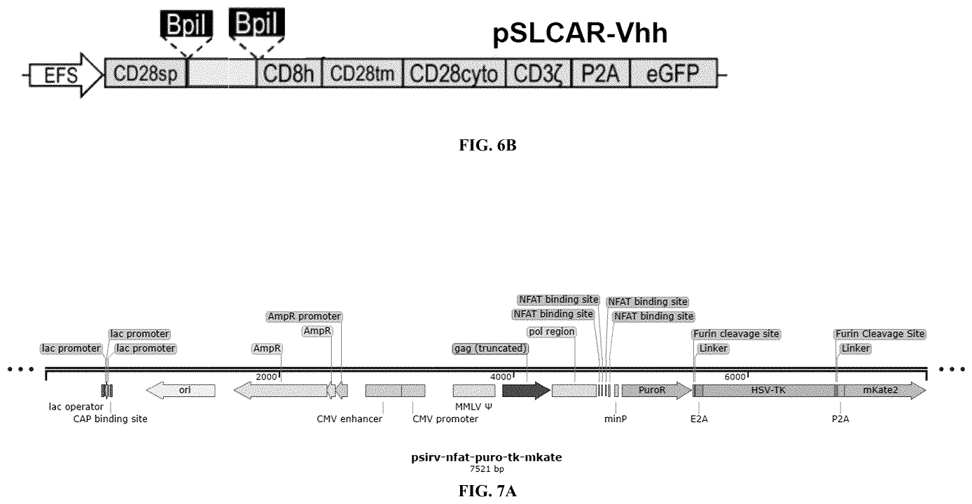

What follows is the nucleotide sequence of the chimeric antigen receptor (CAR) plasmid (pSLCAR-VHH) used for cloning of VHH domains, where the nucleotide sequence encoding a chimeric antigen receptor polypeptide linked to enhanced green fluorescent protein (eGFP) polypeptide by way of a self-cleaving peptide (P2A), and prior to insertion of an anti-mesothelin VHH domain sequence, is shown in BOLD UPPERCASE TEXT. In the below sequence, sites recognized by the Bpil restriction enzyme are shown as BOLD, UPPERCASE, UNDERLINED TEXT. A map of the plasmid is provided at FIGS. 6A and 6B, and Tables 6A and 6B provide amino acid and polynucleotide sequences corresponding to the features labeled in the plasmid map.

(SEQ ID NO: 131)
gacattgattattgactagttattaatagtaatcaattacggggtcattagttcatagccca
tatatggagttccgcgttacataacttacggtaaatggcccgcctggctgaccgcccaacga
cccccgcccattgacgtcaataatgacgtatgttcccatagtaacgccaatagggactttcc
attgacgtcaatgggtggagtatttacggtaaactgcccacttggcagtacatcaagtgtat
catatgccaagtacgccccctattgacgtcaatgacggtaaatggcccgcctggcattatgc
ccagtacatgaccttatgggactttcctacttggcagtacatctacgtattagtcatcgcta
ttaccatggtgatgcggttttggcagtacatcaatgggcgtggatagcggtttgactcacgg
ggatttccaagtctccaccccattgacgtcaatgggagtttgttttggcaccaaaatcaacg
ggactttccaaaatgtcgtaacaactccgccccattgacgcaaatgggcggtaggcgtgtac
ggtgggaggtctatataagcagcgcgttttgcctgtactgggtctctctggttagaccagat
ctgagcctgggagctctctggctaactagggaacccactgcttaagcctcaataaagcttgc
cttgagtgcttcaagtagtgtgtgcccgtctgttgtgtgactctggtaactagagatccctc
agacccttttagtcagtgtggaaaatctctagcagtggcgcccgaacagggacttgaaagcg
aaagggaaaccagaggagctctctcgacgcaggactcggcttgctgaagcgcgcacggcaag
aggcgaggggcggcgactggtgagtacgccaaaaattttgactagcggaggctagaaggaga
gagatgggtgcgagagcgtcagtattaagcgggggagaattagatcgcgatgggaaaaaatt
cggttaaggccagggggaaagaaaaaatataaattaaaacatatagtatgggcaagcaggga
gctagaacgattcgcagttaatcctggcctgttagaaacatcagaaggctgtagacaaatac
tgggacagctacaaccatcccttcagacaggatcagaagaacttagatcattatataataca
gtagcaaccctctattgtgtgcatcaaaggatagagataaaagacaccaaggaagctttaga
caagatagaggaagagcaaaacaaaagtaagaccaccgcacagcaagcggccgctgatcttc
agacctggaggaggagatatgagggacaattggagaagtgaattatataaatataaagtagt
aaaaattgaaccattaggagtagcacccaccaaggcaaagagaagagtggtgcagagagaaa
aaagagcagtgggaataggagctttgttccttgggttcttgggagcagcaggaagcactatg
ggcgcagcgtcaatgacgctgacggtacaggccagacaattattgtctggtatagtgcagca
gcagaacaatttgctgagggctattgaggcgcaacagcatctgttgcaactcacagtctggg
gcatcaagcagctccaggcaagaatcctggctgtggaaagatacctaaaggatcaacagctc
ctggggatttggggttgctctggaaaactcatttgcaccactgctgtgccttggaatgctag
ttggagtaataaatctctggaacagatttggaatcacacgacctggatggagtgggacagag
aaattaacaattacacaagcttaatacactccttaattgaagaatcgcaaaaccagcaagaa
aagaatgaacaagaattattggaattagataaatgggcaagtttgtggaattggtttaacat
aacaaattggctgtggtatataaaattattcataatgatagtaggaggcttggtaggtttaa
gaatagtttttgctgtactttctatagtgaatagagttaggcagggatattcaccattatcg
tttcagacccacctcccaaccccgaggggaccccgggtttattacagggacagcagagatcc
actttggcgccggctcgagttttaaaagaaaaggggggattggggggtacagtgcaggggaa
agaatagtagacataatagcaacagacatacaaactaaagaattacaaaaacaaattacaaa
aattcaaaatttttcgagtggctccggtgcccgtcagtgggcagagcgcacatcgcccacag
tccccgagaagttggggggaggggtcggcaattgaaccggtgcctagagaaggtggcgcggg
gtaaactgggaaagtgatgtcgtgtactggctccgcctttttcccgagggtgggggagaacc
gtatataagtgcagtagtcgccgtgaacgttctttttcgcaacgggtttgccgccagaacac
aggtgtcgtgacgcgggatccgccaccATGCTCAGGCTGCTCTTGGCTCTCAACTTATTCCC
TTCAATTCAAGTAACAGGAGGGTCTTCCTTTTTTGAAGACCCGACGCCAGCGCCAACTATTG
CGAGTCAGCCTCTCAGTCTGCGACCTGAGGCTTGTCGACCAGCAGCCGGAGGCGCAGTGCAC
ACGAGGGGGCTGGACTTCGCCTGTGATCCTTCTAAGCCCTTTTGGGTGCTGGTGGTGGTTGG
TGGAGTCCTGGCTTGCTATAGCTTGCTAGTAACAGTGGCCTTTATTATTTTCTGGGTGAGGA
AACGGGGCAGAAAGAAACTCCTGTATATATTCAAACAACCATTTATGAGACCAGTACAAACT
ACTCAAGAGGAAGATGGCTGTAGCTGCCGATTTCCAGAAGAAGAAGAAGGAGGATGTGAACT
GGCTAGCCTGAGAGTGAAGTTCAGCAGGAGCGCAGACGCCCCCGCGTACCAGCAGGGCCAGA
ACCAGCTCTATAACGAGCTCAATCTAGGACGAAGAGAGGAGTACGATGTTTTGGACAAGAGA
CGTGGCCGGGACCCTGAGATGGGGGGAAAGCCGCAGAGAAGGAAGAACCCTCAGGAAGGCCT
GTACAATGAACTGCAGAAAGATAAGATGGCGGAGGCCTACAGTGAGATTGGGATGAAAGGCG
AGCGCCGGAGGGGCAAGGGGCACGATGGCCTTTACCAGGGTCTCAGTACAGCCACCAAGGAC
ACCTACGACGCCCTTCACATGCAGGCCCTGCCCCCTCGCGCTAGCGCCACGAACTTCTCTCT
GTTAAAGCAAGCAGGCGACGTGGAAGAAAACCCCGGTCCCGTGAGCAAGGGCGAGGAGCTGT
TCACCGGGGTGGTGCCCATCCTGGTCGAGCTGGACGGCGACGTAAACGGCCACAAGTTCAGC
GTGTCCGGCGAGGGCGAGGGCGATGCCACCTACGGCAAGCTGACCCTGAAGTTCATCTGCAC
CACCGGCAAGCTGCCCGTGCCCTGGCCCACCCTCGTGACCACCCTGACCTACGGCGTGCAGT
GCTTCAGCCGCTACCCCGACCACATGAAGCAGCACGACTTCTTCAAGTCCGCCATGCCCGAA
GGCTACGTCCAGGAGCGCACCATCTTCTTCAAGGACGACGGCAACTACAAGACCCGCGCCGA
GGTGAAGTTCGAGGGCGACACCCTGGTGAACCGCATCGAGCTGAAGGGCATCGACTTCAAGG
AGGACGGCAACATCCTGGGGCACAAGCTGGAGTACAACTACAACAGCCACAACGTCTATATC
ATGGCCGACAAGCAGAAGAACGGCATCAAGGTGAACTTCAAGATCCGCCACAACATCGAGGA
CGGCAGCGTGCAGCTCGCCGACCACTACCAGCAGAACACCCCCATCGGCGACGGCCCCGTGC
TGCTGCCCGACAACCACTACCTGAGCACCCAGTCCGCCCTGAGCAAAGACCCCAACGAGAAG
CGCGATCACATGGTCCTGCTGGAGTTCGTGACCGCCGCCGGGATCACTCTCGGCATGGACGA
GCTGTACAAGtaacgcgttaagtcgacaatcaacctctggattacaaaatttgtgaaagatt
gactggtattcttaactatgttgctccttttacgctatgtggatacgctgctttaatgcctt
tgtatcatgctattgcttcccgtatggctttcattttctcctccttgtataaatcctggttg
ctgtctctttatgaggagttgtggcccgttgtcaggcaacgtggcgtggtgtgcactgtgtt
tgctgacgcaacccccactggttggggcattgccaccacctgtcagctcctttccgggactt
tcgctttccccctccctattgccacggcggaactcatcgccgcctgccttgcccgctgctgg
acaggggctcggctgttgggcactgacaattccgtggtgttgtcggggaaatcatcgtcctt
tccttggctgctcgcctgtgttgccacctggattctgcgcgggacgtccttctgctacgtcc
cttcggccctcaatccagcggaccttccttcccgcggcctgctgccggctctgcggcctctt
ccgcgtctttgccttcgccctcagacgagtcggatctccctttgggccgcctccccgcgtcg
actttaagaccaatgacttacaaggcagctgtagatcttagccactttttaaaagaaaaggg
gggactggaagggctaattcactcccaacgaagataagatctgctttttgcttgtactgggt
ctctctggttagaccagatctgagcctgggagctctctggctaactagggaacccactgctt
aagcctcaataaagcttgccttgagtgcttcaagtagtgtgtgcccgtctgttgtgtgactc
tggtaactagagatccctcagacccttttagtcagtgtggaaaatctctagcagtacgtata
gtagttcatgtcatcttattattcagtatttataacttgcaaagaaatgaatatcagagagt
gagaggaacttgtttattgcagcttataatggttacaaataaagcaatagcatcacaaattt
cacaaataaagcatttttttcactgcattctagttgtggtttgtccaaactcatcaatgtat
cttatcatgtctggctctagctatcccgcccctaactccgcccatcccgcccctaactccgc
ccagttccgcccattctccgccccatggctgactaattttttttatttatgcagaggccgag
gccgcctcggcctctgagctattccagaagtagtgaggaggcttttttggaggcctagggac
gtacccaattcgccctatagtgagtcgtattacgcgcgctcactggccgtcgttttacaacg
tcgtgactgggaaaaccctggcgttacccaacttaatcgccttgcagcacatccccctttcg
ccagctggcgtaatagcgaagaggcccgcaccgatcgcccttcccaacagttgcgcagcctg
aatggcgaatgggacgcgccctgtagcggcgcattaagcgcggcgggtgtggtggttacgcg
cagcgtgaccgctacacttgccagcgccctagcgcccgctcctttcgctttcttcccttcct
ttctcgccacgttcgccggctttccccgtcaagctctaaatcgggggctccctttagggttc
cgatttagtgctttacggcacctcgaccccaaaaaacttgattagggtgatggttcacgtag
tgggccatcgccctgatagacggtttttcgccctttgacgttggagtccacgttctttaata
gtggactcttgttccaaactggaacaacactcaaccctatctcggtctattcttttgattta
taagggattttgccgatttcggcctattggttaaaaaatgagctgatttaacaaaaatttaa
cgcgaattttaacaaaatattaacgcttacaatttaggtggcacttttcggggaaatgtgcg
cggaacccctatttgtttatttttctaaatacattcaaatatgtatccgctcatgagacaat
aaccctgataaatgcttcaataatattgaaaaaggaagagtatgagtattcaacatttccgt
gtcgcccttattcccttttttgcggcattttgccttcctgtttttgctcacccagaaacgct
ggtgaaagtaaaagatgctgaagatcagttgggtgcacgagtgggttacatcgaactggatc
tcaacagcggtaagatccttgagagttttcgccccgaagaacgttttccaatgatgagcact
tttaaagttctgctatgtggcgcggtattatcccgtattgacgccgggcaagagcaactcgg
tcgccgcatacactattctcagaatgacttggttgagtactcaccagtcacagaaaagcatc
ttacggatggcatgacagtaagagaattatgcagtgctgccataaccatgagtgataacact
gcggccaacttacttctgacaacgatcggaggaccgaaggagctaaccgcttttttgcacaa
catgggggatcatgtaactcgccttgatcgttgggaaccggagctgaatgaagccataccaa
acgacgagcgtgacaccacgatgcctgtagcaatggcaacaacgttgcgcaaactattaact
ggcgaactacttactctagcttcccggcaacaattaatagactggatggaggcggataaagt
tgcaggaccacttctgcgctcggcccttccggctggctggtttattgctgataaatctggag
ccggtgagcgtgggtctcgcggtatcattgcagcactggggccagatggtaagccctcccgt
atcgtagttatctacacgacggggagtcaggcaactatggatgaacgaaatagacagatcgc
tgagataggtgcctcactgattaagcattggtaactgtcagaccaagtttactcatatatac
tttagattgatttaaaacttcatttttaatttaaaaggatctaggtgaagatcctttttgat
aatctcatgaccaaaatcccttaacgtgagttttcgttccactgagcgtcagaccccgtaga
aaagatcaaaggatcttcttgagatcctttttttctgcgcgtaatctgctgcttgcaaacaa
aaaaaccaccgctaccagcggtggtttgtttgccggatcaagagctaccaactctttttccg
aaggtaactggcttcagcagagcgcagataccaaatactgttcttctagtgtagccgtagtt
aggccaccacttcaagaactctgtagcaccgcctacatacctcgctctgctaatcctgttac
cagtggctgctgccagtggcgataagtcgtgtcttaccgggttggactcaagacgatagtta
ccggataaggcgcagcggtcgggctgaacggggggttcgtgcacacagcccagcttggagcg
aacgacctacaccgaactgagatacctacagcgtgagctatgagaaagcgccacgcttcccg
aagggagaaaggcggacaggtatccggtaagcggcagggtcggaacaggagagcgcacgagg
gagcttccagggggaaacgcctggtatctttatagtcctgtcgggtttcgccacctctgact
tgagcgtcgatttttgtgatgctcgtcaggggggcggagcctatggaaaaacgccagcaacg
cggcctttttacggttcctggccttttgctggccttttgctcacatgtgtcgacggatcggg
agatctcccgatcccctatggtgcactctcagtacaatctgctctgatgccgcatagttaag
ccagtatctgctccctgcttgtgtgttggaggtcgctgagtagtgcgcgagcaaaatttaag
ctacaacaaggcaaggcttgaccgacaattgcatgaagaatctgcttagggttaggcgtttt
gcgctgcttcgcgatgtacgggccagatatacgcgtt

What follows is the amino acid sequence encoded by the BOLD ALL CAPS sequence shown above, which corresponds to a CAR sequence lacking an antigen binding domain (e.g., a VHH domain) that is linked to enhanced green fluorescent protein (eGFP) polypeptide by way of a self-cleaving peptide (P2A).

(SEQ ID NO: 132)
MLRLLLALNLFPSIQVTGGSSFFEDPTPAPTIASQPLSLRPEACRPAAGGAVHTRGLDFACD
PSKPFWVLVVVGGVLACYSLLVTVAFIIFWVRKRGRKKLLYIFKQPFMRPVQTTQEEDGCSC
RFPEEEEGGCELASLRVKFSRSADAPAYQQGQNQLYNELNLGRREEYDVLDKRRGRDPEMGG
KPQRRKNPQEGLYNELQKDKMAEAYSEIGMKGERRRGKGHDGLYQGLSTATKDTYDALHMQA
LPPRASATNFSLLKQAGDVEENPGPVSKGEELFTGVVPILVELDGDVNGHKFSVSGEGEGDA
TYGKLTLKFICTTGKLPVPWPTLVTTLTYGVQCFSRYPDHMKQHDFFKSAMPEGYVQERTIF
FKDDGNYKTRAEVKFEGDTLVNRIELKGIDFKEDGNILGHKLEYNYNSHNVYIMADKQKNGI
KVNFKIRHNIEDGSVQLADHYQQNTPIGDGPVLLPDNHYLSTQSALSKDPNEKRDHMVLLEF
VTAAGITLGMDELYK

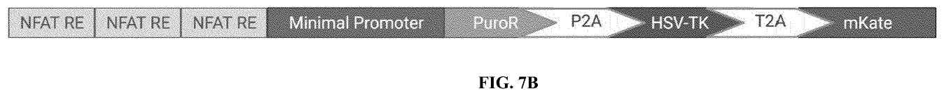

What follows is the nucleotide sequence of the plasmid encoding the activation reporter cassette used in the Examples. A map of the plasmid is provided at FIGS. 7A and 7B, and Tables 6A and 6B provide amino acid and polynucleotide sequences corresponding to the features labeled in the plasmid map.

(SEQ ID NO: 133)
ggccgccagcacagtggtcgatcgacgataaaataaaagattttatttagtctccagaaaaa
ggggggaatgaaagaccccacctgtaggtttggcaagctagcttaagtaacgccattttgca
aggcatggaaaaatacataactgagaatagaaaagttcagatcaaggtcaggaacagatgga
acagggtcgcgtcccgcaataaaagagcccacaacccctcactcggggcgccagtcctccga
ttgactgagtcgcccgggtacccgtgtatccaataaaccctcttgcagttgcatccgacttg
tggtctcgctgttccttgggagggtctcctctgagtgattgactacccgtcagcgggggtct
ttcacatgcagcatgtatcaaaattaatttggttttttttcttaagtatttacattaaatgg
ccatagtagttcattatggacagcgcagaaagagctggggagaattgtgaaattgttatccg
ctcacaattccacacaacatacgagccggaagcataaagtgtaaagcctggggtgcctaatg
agtgagctaactcacattaattgcgttgcgctcactgcccgctttccagtcgggaaacctgt
cgtgccagctgcattaatgaatcggccaacgcgcggggagaggcggtttgcgtattgggcgc
tcttccgcttcctcgctcactgactcgctgcgctcggtcgttcggctgcggcgagcggtatc
agctcactcaaaggcggtaatacggttatccacagaatcaggggataacgcaggaaagaaca
tgtgagcaaaaggccagcaaaaggccaggaaccgtaaaaaggccgcgttgctggcgtttttc
cataggctccgcccccctgacgagcatcacaaaaatcgacgctcaagtcagaggtggcgaaa
cccgacaggactataaagataccaggcgtttccccctggaagctccctcgtgcgctctcctg
ttccgaccctgccgcttaccggatacctgtccgcctttctcccttcgggaagcgtggcgctt
tctcatagctcacgctgtaggtatctcagttcggtgtaggtcgttcgctccaagctgggctg
tgtgcacgaaccccccgttcagcccgaccgctgcgccttatccggtaactatcgtcttgagt
ccaacccggtaagacacgacttatcgccactggcagcagccactggtaacaggattagcaga
gcgaggtatgtaggcggtgctacagagttcttgaagtggtggcctaactacggctacactag
aagaacagtatttggtatctgcgctctgctgaagccagttaccttcggaaaaagagttggta
gctcttgatccggcaaacaaaccaccgctggtagcggtggtttttttgtttgcaagcagcag
attacgcgcagaaaaaaaggatctcaagaagatcctttgatcttttctacggggtctgacgc
tcagtggaacgaaaactcacgttaagggattttggtcatgagattatcaaaaaggatcttca
cctagatccttttgcggccggccgcaaatcaatctaaagtatatatgagtaaacttggtctg
acagttaccaatgcttaatcagtgaggcacctatctcagcgatctgtctatttcgttcatcc
atagttgcctgactccccgtcgtgtagataactacgatacgggagggcttaccatctggccc
cagtgctgcaatgataccgcgagacccacgctcaccggctccagatttatcagcaataaacc
agccagccggaagggccgagcgcagaagtggtcctgcaactttatccgcctccatccagtct
attaattgttgccgggaagctagagtaagtagttcgccagttaatagtttgcgcaacgttgt
tgccattgctacaggcatcgtggtgtcacgctcgtcgtttggtatggcttcattcagctccg
gttcccaacgatcaaggcgagttacatgatcccccatgttgtgcaaaaaagcggttagctcc
ttcggtcctccgatcgttgtcagaagtaagttggccgcagtgttatcactcatggttatggc
agcactgcataattctcttactgtcatgccatccgtaagatgcttttctgtgactggtgagt
actcaaccaagtcattctgagaatagtgtatgcggcgaccgagttgctcttgcccggcgtca
atacgggataataccgcgccacatagcagaactttaaaagtgctcatcattggaaaacgttc
ttcggggcgaaaactctcaaggatcttaccgctgttgagatccagttcgatgtaacccactc
gtgcacccaactgatcttcagcatcttttactttcaccagcgtttctgggtgagcaaaaaca
ggaaggcaaaatgccgcaaaaaagggaataagggcgacacggaaatgttgaatactcatact
cttcctttttcaatattattgaagcatttatcagggttattgtctcatgagcggatacatat
ttgaatgtatttagaaaaataaacaaataggggttccgcgcacatttccccgaaaagtgcca
ccagctttgctcttaggagtttcctaatacatcccaaactcaaatatataaagcatttgact
tgttctatgccctagttattaatagtaatcaattacggggtcattagttcatagcccatata
tggagttccgcgttacataacttacggtaaatggcccgcctggctgaccgcccaacgacccc
cgcccattgacgtcaataatgacgtatgttcccatagtaacgccaatagggactttccattg
acgtcaatgggtggagtatttacggtaaactgcccacttggcagtacatcaagtgtatcata
tgccaagtacgccccctattgacgtcaatgacggtaaatggcccgcctggcattatgcccag
tacatgaccttatgggactttcctacttggcagtacatctacgtattagtcatcgctattac
catggtgatgcggttttggcagtacatcaatgggcgtggatagcggtttgactcacggggat
ttccaagtctccaccccattgacgtcaatgggagtttgttttggcaccaaaatcaacgggac
tttccaaaatgtcgtaacaactccgccccattgacgcaaatgggcggtaggcgtgtacggtg
ggaggtctatataagcagagctcaataaaagagcccacaacccctcactcggcgcgccagtc
ctccgattgactgagtcgcccgggtacccgtgtatccaataaaccctcttgcagttgcatcc
gacttgtggtctcgctgttccttgggagggtctcctctgagtgattgactacccgtcagcgg
gggtctttcatttgggggctcgtccgggatcgggagacccctgcccagggaccaccgaccca
ccaccgggaggtaagctggccagcaacttatctgtgtctgtccgattgtctagtgtctatga
ctgattttatgcgcctgcgtcggtactagttagctaactagctctgtatctggcggacccgt
ggtggaactgacgagttcggaacacccggccgcaaccctgggagacgtcccagggacttcgg
gggccgtttttgtggcccgacctgagtccaaaaatcccgatcgttttggactctttggtgca
ccccccttagaggagggatatgtggttctggtaggagacgagaacctaaaacagttcccgcc
tccgtctgaatttttgctttcggtttgggaccgaagccgcgccgcgcgtcttgtctgctgca
gcatcgttctgtgttgtctctgtctgactgtgtttctgtatttgtctgaaaatatgggcccg
ggccagactgttaccactcccttaagtttgaccttaggtcactggaaagatgtcgagcggat
cgctcacaaccagtcggtagatgtcaagaagagacgttgggttaccttctgctctgcagaat
ggccaacctttaacgtcggatggccgcgagacggcacctttaaccgagacctcatcacccag
gttaagatcaaggtcttttcacctggcccgcatggacacccagaccaggtcccctacatcgt
gacctgggaagccttggcttttgacccccctccctgggtcaagccctttgtacaccctaagc
ctccgcctcctcttcctccatccgccccgtctctcccccttgaacctcctcgttcgaccccg
cctcgatcctccctttatccagccctcactccttctctaggcgcccccatatggccatatga
gatcttatatggggcacccccgccccttgtaaacttccctgaccctgacatgacaagagtta
ctaacagcccctctctccaagctcacttacaggctctctacttagtccagcacgaagtctgg
agacctctggcggcagcctaccaagaacaactggaccgaccggtggtacctcacccttaccg
agtcggcgacacagtgtgggtccgccgacaccagactaagaacctagaacctcgctggaaag
gaccttacacagtcctgctgaccacccccaccgccctcaaagtagacggcatcgcagcttgg
atacacgccgcccacgtgaaggctgccgaccccgggggtggaccatcctctagactgccgga
tccaagctggaggaaaaactgtttcatacagaaggcgtggaggaaaaactgtttcatacaga
aggcgtggaggaaaaactgtttcatacagaaggcgtggaggaaaaactgtttcatacagaag
gcgtcgcgaattcgcggagactctagagggtatataatggaagctcgatttccagcttggca
ttccggtactgttggtaaacaccaagctatgaccgagtacaagcccacggtgcgcctcgcca
cccgcgacgacgtccccagggccgtacgcaccctcgccgccgcgttcgccgactaccccgcc
acgcgccacaccgtcgatccggaccgccacatcgagcgggtcaccgagctgcaagaactctt
cctcacgcgcgtcgggctcgacatcggcaaggtgtgggtcgcggacgacggcgccgccgtgg
cggtctggaccacgccggagagcgtcgaagcgggggcggtgttcgccgagatcggcccgcgc
atggccgagttgagcggttcccggctggccgcgcagcaacagatggaaggcctcctggcgcc
gcaccggcccaaggagcccgcgtggttcctggccaccgtcggcgtttcgcccgaccaccagg
gcaagggtctgggcagcgccgtcgtgctccccggagtggaggcggccgagcgcgccggggtg
cccgccttcctggagacctccgcgccccgcaacctccccttctacgagcggctcggcttcac
cgtcaccgccgacgtcgaggtgcccgaaggaccgcgcacctggtgcatgacccgcaagcccg
gtgccgctagccgggccaagcggtccggatccggacagtgcaccaactatgcgctgctgaaa
ctggcgggcgatgtggaaagcaacccgggcccgatggcttcgtacccctgccatcaacacgc
gtctgcgttcgaccaggctgcgcgttctcgcggccatagcaaccgacgtacggcgttgcgcc
ctcgccggcagcaagaagccacggaagtccgcctggagcagaaaatgcccacgctactgcgg
gtttatatagacggtcctcacgggatggggaaaaccaccaccacgcaactgctggtggccct
gggttcgcgcgacgatatcgtctacgtacccgagccgatgacttactggcaggtgctggggg
cttccgagacaatcgcgaacatctacaccacacaacaccgcctcgaccagggtgagatatcg
gccggggacgcggcggtggtaatgacaagcgcccagataacaatgggcatgccttatgccgt
gaccgacgccgttctggctcctcatatcgggggggaggctgggagctcacatgccccgcccc
cggccctcaccctcatcttcgaccgccatcccatcgccgccctcctgtgctacccggccgcg
cgataccttatgggcagcatgaccccccaggccgtgctggcgttcgtggccctcatcccgcc
gaccttgcccggcacaaacatcgtgttgggggcccttccggaggacagacacatcgaccgcc
tggccaaacgccagcgccccggcgagcggcttgacctggctatgctggccgcgattcgccgc
gtttacgggctgcttgccaatacggtgcggtatctgcagggcggcgggtcgtggcgggagga
ttggggacagctttcggggacggccgtgccgccccagggtgccgagccccagagcaacgcgg
gcccacgaccccatatcggggacacgttatttaccctgtttcgggcccccgagttgctggcc
cccaacggcgacctgtacaacgtgtttgcctgggccttggacgtcttggccaaacgcctccg
tcccatgcacgtctttatcctggattacgaccaatcgcccgccggctgccgggacgccctgc
tgcaacttacctccgggatggtccagacccacgtcaccacccccggctccataccgacgatc
tgcgacctggcgcgcacgtttgcccgggagatgggggaggctaaccgcgccaagcgctcggg
ttcgggtgccaccaacttcagcctgctgaagcaggccggcgacgtggaggagaaccccggcc
ccatggtgagcgagctgattaaggagaacatgcacatgaagctgtacatggagggcaccgtg
aacaaccaccacttcaagtgcacatccgagggcgaaggcaagccctacgagggcacccagac
catgagaatcaaggcggtcgagggcggccctctccccttcgccttcgacatcctggctacca
gcttcatgtacggcagcaaaaccttcatcaaccacacccagggcatccccgacttctttaag
cagtccttccccgagggcttcacatgggagagagtcaccacatacgaagacgggggcgtgct
gaccgctacccaggacaccagcctccaggacggctgcctcatctacaacgtcaagatcagag
gggtgaacttcccatccaacggccctgtgatgcagaagaaaacactcggctgggaggcctcc
accgagaccctgtaccccgctgacggcggcctggaaggcagagccgacatggccctgaagct
cgtgggcgggggccacctgatctgcaacttgaagaccacatacagatccaagaaacccgcta
agaacctcaagatgcccggcgtctactatgtggacagaagactggaaagaatcaaggaggcc
gacaaagagacctacgtcgagcagcacgaggtggctgtggccagatactgcgacctccctag
caaactggggcacagataa

Lengthy table referenced here
US20260125465A1-20260507-T00001
Please refer to the end of the specification for access instructions.

Lengthy table referenced here
US20260125465A1-20260507-T00002
Please refer to the end of the specification for access instructions.

Lengthy table referenced here
US20260125465A1-20260507-T00003
Please refer to the end of the specification for access instructions.

Lengthy table referenced here
US20260125465A1-20260507-T00004
Please refer to the end of the specification for access instructions.

Lengthy table referenced here
US20260125465A1-20260507-T00005
Please refer to the end of the specification for access instructions.

Lengthy table referenced here
US20260125465A1-20260507-T00006
Please refer to the end of the specification for access instructions.

Lengthy table referenced here
US20260125465A1-20260507-T00007
Please refer to the end of the specification for access instructions.

Lengthy table referenced here
US20260125465A1-20260507-T00008
Please refer to the end of the specification for access instructions.

Lengthy table referenced here
US20260125465A1-20260507-T00009
Please refer to the end of the specification for access instructions.

Lengthy table referenced here
US20260125465A1-20260507-T00010
Please refer to the end of the specification for access instructions.

Lengthy table referenced here
US20260125465A1-20260507-T00011
Please refer to the end of the specification for access instructions.

Lengthy table referenced here
US20260125465A1-20260507-T00012
Please refer to the end of the specification for access instructions.

Lengthy table referenced here
US20260125465A1-20260507-T00013
Please refer to the end of the specification for access instructions.

Lengthy table referenced here
US20260125465A1-20260507-T00014
Please refer to the end of the specification for access instructions.

Lengthy table referenced here
US20260125465A1-20260507-T00015
Please refer to the end of the specification for access instructions.

Lengthy table referenced here
US20260125465A1-20260507-T00016
Please refer to the end of the specification for access instructions.

Lengthy table referenced here
US20260125465A1-20260507-T00017
Please refer to the end of the specification for access instructions.

Lengthy table referenced here
US20260125465A1-20260507-T00018
Please refer to the end of the specification for access instructions.

Lengthy table referenced here
US20260125465A1-20260507-T00019
Please refer to the end of the specification for access instructions.

Lengthy table referenced here
US20260125465A1-20260507-T00020
Please refer to the end of the specification for access instructions.

Lengthy table referenced here
US20260125465A1-20260507-T00021
Please refer to the end of the specification for access instructions.

Lengthy table referenced here
US20260125465A1-20260507-T00022
Please refer to the end of the specification for access instructions.

Lengthy table referenced here
US20260125465A1-20260507-T00023
Please refer to the end of the specification for access instructions.

Lengthy table referenced here
US20260125465A1-20260507-T00024
Please refer to the end of the specification for access instructions.

Lengthy table referenced here
US20260125465A1-20260507-T00025
Please refer to the end of the specification for access instructions.

### OTHER EMBODIMENTS

From the foregoing description, it will be apparent that variations and modifications may be made to the various aspects and embodiments described herein to adapt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

LENGTHY TABLES
The patent application contains a lengthy table section. A copy of the table is available in electronic form from the USPTO web site (https://seqdata.uspto.gov/docdetail?docId=US20260125465A1). An electronic copy of the table will also be available from the USPTO upon request and payment of the fee set forth in 37 CFR 1.19(b)(3).

What is claimed is:

1. A VH-heavy chain only (VHH) polypeptide or an antigen binding portion thereof that specifically binds to mesothelin, wherein the binding protein or the antigen binding portion thereof comprises three Complementarity Determining Regions (CDRs): CDR1, CDR2 and CDR3, which are structurally positioned between four camelid VHH framework (FR) regions (FR1-FR4) as follows: FR1-CDR1-FR2-CDR2-FR3-CDR3-FR4; wherein the three CDRs are selected from:
CDR1 comprising the amino acid sequence GRTFSSYA (SEQ ID NO: 1); CDR2 comprising the amino acid sequence ICWSGGNT (SEQ ID NO: 2); and CDR3 comprising the amino acid sequence NERTTVLPTGAMDY (SEQ ID NO: 3);
CDR1 comprising the amino acid sequence GSMFSINA (SEQ ID NO: 4); CDR2 comprising the amino acid sequence MTSGGST (SEQ ID NO: 5); and CDR3 comprising the amino acid sequence NGGWSLAMDY (SEQ ID NO: 6);
CDR1 comprising the amino acid sequence GFTLDYYA (SEQ ID NO: 7); CDR2 comprising the amino acid sequence ISSSGGST (SEQ ID NO: 8); and CDR3 comprising the amino acid sequence AAAGGYGNSSRY (SEQ ID NO: 9);
CDR1 comprising the amino acid sequence GSIFSVNA (SEQ ID NO: 10); CDR2 comprising the amino acid sequence INTGGGST (SEQ ID NO: 11); and CDR3 comprising the amino acid sequence AAEDFGNSKTY (SEQ ID NO: 12);
CDR1 comprising the amino acid sequence GFPYFSCC (SEQ ID NO: 13); CDR2 comprising the amino acid sequence ISSDGST (SEQ ID NO: 14); and CDR3 comprising the amino acid sequence AASPRQLGPYYAMDY (SEQ ID NO: 15);
CDR1 comprising the amino acid sequence GFTLDNYA (SEQ ID NO: 16); CDR2 comprising the amino acid sequence ISSSGGST (SEQ ID NO: 8); and CDR3 comprising the amino acid sequence AGVGDYGRRA (SEQ ID NO: 17); and
CDR1 comprising the amino acid sequence GFTFDDSD (SEQ ID NO: 18); CDR2 comprising the amino acid sequence ISSDGST (SEQ ID NO: 14); and CDR3 comprising the amino acid sequence RTMASRIGC (SEQ ID NO: 19).

2. The VHH polypeptide of claim 1, wherein
i) FR1 comprises the following amino acid sequence:
X₁X₂QLVESGGGX₃VQX₄GX₅X₆LRLX₇CX₈AS (SEQ ID NO: 20), wherein
X₁ is E, D, or Q,
X₂ is V or L,
X₃ is L or S,
X₄ is A or P,
X₅ is E or G,
X₆ is S or T,
X₇ is S or T, and
X₈ is A, T, or V;
ii) FR2 comprises the following amino acid sequence:
XₐX_{b}X₁₃X₁₄RQX₁₅X₁₆X₁₇X₁₈X₁₉X₂₀X₂₁X₂₂VX₂₃X_{c}, wherein
Xₐ is I, M, or V,
X_{b} is A, G, or S,
X₁₃ is W or Y,
X₁₄ is F, H, or Y,
X₁₅ is A, I, or X,
X₁₆ is P or S,
X₁₇ is G or T,
X₁₈ is D, K, or E,
X₁₉ is D, E, or Q,
X₂₀ is C or R,
X₂₁ is E or N,
X₂₂ is F, G, L, or P,
X₂₃ is S or A, and
X_{c} is A, S, T, or Y;
iii) FR3 comprises the following amino acid sequence:
X_{d}YAXₑX_{f}VX_{g}GRFTX₂₄SX₂₅X₂₆X₂₇AX₂₈X₂₉X₃₀X₃₁X₃₂LQMX₃₃SLKX₃₄EDT X₃₅X₃₆YX₃₇C (SEQ ID NO: 21), wherein
X_{d} is F, H, I, N, or Y,
Xₑ is A or D,
X_{f} is S or T,
X_{g} is K or R,
X₂₄ is I or V,
X₂₅ is G, K, R, Q, or W,
X₂₆ is D or G,
X₂₇ is N or T,
X₂₈ is K, N, or R,
X₂₉ is N, H, or Q,
X₃₀ is T or S,
X₃₁ is V or L,
X₃₂ is F or Y,
X₃₃ is N, S, or T,
X₃₄ is P or S,
X₃₅ is A or G,
X₃₆ is I or V, and
X₃₇ is F, I, S, or Y; and
iv) FR4 comprises the following amino acid sequence:
X₄₀GQGTX₄₁VTVX₄₂X₄₃ (SEQ ID NO: 22), wherein
X₄₀ is M or W,
X₄₁ is L or S,
X₄₂ is null or S, and
X₄₃ is null or S.

3. The VHH polypeptide of claim 1, wherein the polypeptide binds a mesothelin antigen with a K_{d} of less than 12 nM.

4. A polypeptide that specifically binds to mesothelin, wherein the polypeptide or a mesothelin-binding portion thereof has at least 85% amino acid sequence identity to a
(SEQ ID NO: 55)
QVQLVESGGGLVQAGGSLRLSCVASGRTFSSYAMGWFRQSPGKEREFVA
AICWSGGNTYYADSVKGRFTISGDNAKNSVYLQMNSLKPEDTAVYICNE
RTTVLPTGAMDYWGQGTSVTVSS;
(SEQ ID NO: 56)
QVQLVESGGGLVQPGGSLRLSCAASGSMFSINAVAWYRQAPGEQREPVA
AMTSGGSTIYADSVKGRFTISRDNAKNTVYLQMSSLKPEDTAVYYCNGG
WSLAMDYWGQGTSVTVS;
(SEQ ID NO: 57)
QLQLVESGGGLVQPGGSLRLSCAASGFTLDYYAIGWFRQAPGKEREGVS
YISSSGGSTNYADSVKGRFTISRDNAKNTVYLQMNSLKPEDTGVYSCAA
AGGYGNSSRYWGQGTSVTVS;
(SEQ ID NO: 58)
QVQLVESGGGLVQPGGSLRLSCAASGSIFSVNAMGYYRQISTKQRNLVA
AINTGGGSTYYADSVKGRFTISWDNANNTLFLQMNSLKSEDTAVYYCAA
EDFGNSKTYWGQGTLVTVS;
(SEQ ID NO: 4476)
DVQLVESGGGLVQAGGSLRLSCAASGFPYFSCCMSWHRQAPGKDRELVS
SISSDGSTHYADSVRGRFTISKGNARNTLYLQMNSLKPEDTAVYFCAAS
PRQLGPYYAMDYWGQGTSVTVS;
(SEQ ID NO: 60)
QLQLVESGGGLVQPGGSLRLSCAASGFTLDNYAIGWFRQAPGKEREGVS
SISSSGGSTNYADSVKGRFTVSRDNAKHTVYLQMNSLKPEDTAVYYCAG
VGDYGRRAMGQGTSVTVS;
(SEQ ID NO: 61)
EVQLVESGGGSVQAGETLRLSCTASGFTFDDSDMGWYRQAPGDECELVS
TISSDGSTNYADSVKGRFTISQDNAKNTVYLQMTSLKPEDTAIYSCRTM
ASRIGCWGQGTLVTVS.

5. A chimeric antigen receptor polypeptide (CAR) comprising the polypeptide of claim 1.

6. The CAR of claim 5, wherein the polypeptide comprises an amino acid sequence with at least about 85% identity to one of the following sequences:
(SEQ ID NO: 23)
MLRLLLALNLFPSIQVTGGQVQLVESGGGLVQPGGSLRLSCAASGSMFSINAVAWYRQAPGE
QREPVAAMTSGGSTIYADSVKGRFTISRDNAKNTVYLQMSSLKPEDTAVYYCNGGWSLAMDY
WGQGTSVTVSPTPAPTIASQPLSLRPEACRPAAGGAVHTRGLDFACDPSKPFWVLVVVGGVL
ACYSLLVTVAFIIFWVRKRGRKKLLYIFKQPFMRPVQTTQEEDGCSCRFPEEEEGGCELASL
RVKFSRSADAPAYQQGQNQLYNELNLGRREEYDVLDKRRGRDPEMGGKPQRRKNPQEGLYNE
LQKDKMAEAYSEIGMKGERRRGKGHDGLYQGLSTATKDTYDALHMQALPPRAS;
(SEQ ID NO: 24)
MLRLLLALNLFPSIQVTGGQVQLVESGGGLVQPGGSLRLSCAASGSIFSVNAMGYYRQISTK
QRNLVAAINTGGGSTYYADSVKGRFTISWDNANNTLFLQMNSLKSEDTAVYYCAAEDFGNSK
TYWGQGTLVTVSPTPAPTIASQPLSLRPEACRPAAGGAVHTRGLDFACDPSKPFWVLVVVGG
VLACYSLLVTVAFIIFWVRKRGRKKLLYIFKQPFMRPVQTTQEEDGCSCRFPEEEEGGCELA
SLRVKFSRSADAPAYQQGQNQLYNELNLGRREEYDVLDKRRGRDPEMGGKPQRRKNPQEGLY
NELQKDKMAEAYSEIGMKGERRRGKGHDGLYQGLSTATKDTYDALHMQALPPRAS;
(SEQ ID NO: 25)
MLRLLLALNLFPSIQVTGGDVQLVESGGGLVQAGGSLRLSCAASGFPYFSCCMSWHRQAPGK
DRELVSSISSDGSTHYADSVRGRFTISKGNARNTLYLQMNSLKPEDTAVYFCAASPRQLGPY
YAMDYWGQGTSVTVSPTPAPTIASQPLSLRPEACRPAAGGAVHTRGLDFACDPSKPFWVLVV
VGGVLACYSLLVTVAFIIFWVRKRGRKKLLYIFKQPFMRPVQTTQEEDGCSCRFPEEEEGGC
ELASLRVKFSRSADAPAYQQGQNQLYNELNLGRREEYDVLDKRRGRDPEMGGKPQRRKNPQE
GLYNELQKDKMAEAYSEIGMKGERRRGKGHDGLYQGLSTATKDTYDALHMQALPPRAS;
(SEQ ID NO: 26)
MLRLLLALNLFPSIQVTGGQLQLVESGGGLVQPGGSLRLSCAASGFTLDNYAIGWFRQAPGK
EREGVSSISSSGGSTNYADSVKGRFTVSRDNAKHTVYLQMNSLKPEDTAVYYCAGVGDYGRR
AMGQGTSVTVSPTPAPTIASQPLSLRPEACRPAAGGAVHTRGLDFACDPSKPFWVLVVVGGV
LACYSLLVTVAFIIFWVRKRGRKKLLYIFKQPFMRPVQTTQEEDGCSCRFPEEEEGGCELAS
LRVKFSRSADAPAYQQGQNQLYNELNLGRREEYDVLDKRRGRDPEMGGKPQRRKNPQEGLYN
ELQKDKMAEAYSEIGMKGERRRGKGHDGLYQGLSTATKDTYDALHMQALPPRAS;
(SEQ ID NO: 27)
MLRLLLALNLFPSIQVTGGQLQLVESGGGLVQPGGSLRLSCAASGFTLDYYAIGWFRQAPGK
EREGVSYISSSGGSTNYADSVKGRFTISRDNAKNTVYLQMNSLKPEDTGVYSCAAAGGYGNS
SRYWGQGTSVTVSPTPAPTIASQPLSLRPEACRPAAGGAVHTRGLDFACDPSKPFWVLVVVG
GVLACYSLLVTVAFIIFWVRKRGRKKLLYIFKQPFMRPVQTTQEEDGCSCRFPEEEEGGCEL
ASLRVKFSRSADAPAYQQGQNQLYNELNLGRREEYDVLDKRRGRDPEMGGKPQRRKNPQEGL
YNELQKDKMAEAYSEIGMKGERRRGKGHDGLYQGLSTATKDTYDALHMQALPPRAS;
(SEQ ID NO: 28)
MLRLLLALNLFPSIQVTGGEVQLVESGGGSVQAGETLRLSCTASGFTFDDSDMGWYRQAPGD
ECELVSTISSDGSTNYADSVKGRFTISQDNAKNTVYLQMTSLKPEDTAIYSCRTMASRIGCW
GQGTLVTVSPTPAPTIASQPLSLRPEACRPAAGGAVHTRGLDFACDPSKPFWVLVVVGGVLA
CYSLLVTVAFIIFWVRKRGRKKLLYIFKQPFMRPVQTTQEEDGCSCRFPEEEEGGCELASLR
VKFSRSADAPAYQQGQNQLYNELNLGRREEYDVLDKRRGRDPEMGGKPQRRKNPQEGLYNEL
QKDKMAEAYSEIGMKGERRRGKGHDGLYQGLSTATKDTYDALHMQALPPRAS;
and
(SEQ ID NO: 29)
MLRLLLALNLFPSIQVTGGQVQLVESGGGLVQAGGSLRLSCVASGRTFSSYAMGWFRQSPGK
EREFVAAICWSGGNTYYADSVKGRFTISGDNAKNSVYLQMNSLKPEDTAVYICNERTTVLPT
GAMDYWGQGTSVTVSSPTPAPTIASQPLSLRPEACRPAAGGAVHTRGLDFACDPSKPFWVLV
VVGGVLACYSLLVTVAFIIFWVRKRGRKKLLYIFKQPFMRPVQTTQEEDGCSCRFPEEEEGG
CELASLRVKFSRSADAPAYQQGQNQLYNELNLGRREEYDVLDKRRGRDPEMGGKPQRRKNPQ
EGLYNELQKDKMAEAYSEIGMKGERRRGKGHDGLYQGLSTATKDTYDALHMQALPPRAS.

7. The CAR of claim 5, wherein the CAR comprises in order from N-terminus to C-terminus a CD28 signal peptide, the polypeptide of claim 1, a CD8 hinge domain, a CD28 transmembrane domain, a CD28 cytoplasmic domain, and a CD3ζ domain.

8. A polynucleotide encoding the VHH polypeptide of claim 1.

9. The polynucleotide of claim 8, wherein the polynucleotide comprises a sequence that has at least 85% identity to a sequence selected from the group consisting of.
(SEQ ID NO: 69)
CAAGTTCAGCTTGTGGAGTCAGGCGGTGGACTCGTTCAAGCCGGAGGCTCACTGCGGTTGTC
TTGTGTCGCATCTGGTCGAACCTTCTCAAGTTATGCTATGGGGTGGTTCCGCCAGTCTCCTG
GTAAAGAGCGGGAATTTGTCGCAGCAATTTGTTGGTCTGGTGGTAACACTTACTATGCTGAT
TCAGTAAAAGGTCGATTCACCATATCAGGGGATAATGCAAAAAATTCTGTCTATCTCCAAAT
GAACTCTCTGAAGCCTGAAGATACTGCTGTGTATATCTGTAATGAGAGGACTACGGTACTAC
CTACGGGTGCTATGGACTACTGGGGTCAAGGGACCTCAGTCACCGTCTCCTCG;
(SEQ ID NO: 70)
CAAGTTCAGCTTGTCGAGAGTGGTGGAGGATTGGTGCAGCCAGGCGGATCTCTCAGACTTTC
CTGCGCCGCCAGTGGTTCTATGTTTTCCATAAATGCAGTGGCCTGGTACAGGCAAGCACCAG
GCGAGCAAAGGGAACCGGTAGCCGCAATGACCTCTGGAGGTAGTACAATTTACGCTGATAGC
GTCAAGGGACGATTCACAATATCACGAGACAATGCCAAGAACACTGTCTACCTTCAAATGAG
CAGTTTGAAACCTGAGGATACAGCAGTATACTACTGCAATGGAGGATGGTCACTGGCTATGG
ACTACTGGGGTCAAGGAACCTCAGTCACCGTCTCC;
(SEQ ID NO: 71)
CAGCTTCAACTGGTAGAGAGTGGTGGAGGACTTGTTCAGCCTGGAGGGTCCCTTAGACTTAG
TTGCGCTGCCAGTGGGTTCACCCTGGACTACTACGCCATTGGCTGGTTTCGACAGGCCCCAG
GGAAAGAGCGAGAGGGGGTCTCTTACATCAGTAGTTCCGGTGGTTCCACTAACTACGCTGAT
AGTGTGAAGGGGAGGTTTACTATTTCTCGGGATAATGCAAAAAATACCGTGTACCTGCAAAT
GAATAGCCTCAAACCAGAAGACACAGGCGTTTATTCTTGCGCAGCAGCTGGGGGCTATGGTA
ACTCCTCTCGCTACTGGGGTCAAGGAACCTCAGTCACCGTCTCC;
(SEQ ID NO: 72)
CAGGTTCAGCTGGTGGAGTCCGGCGGAGGTCTTGTTCAGCCCGGAGGTTCCCTGCGCCTCTC
TTGTGCAGCTTCTGGTTCCATTTTCTCAGTCAATGCAATGGGCTACTACAGACAAATTTCCA
CAAAGCAGCGCAACTTGGTCGCCGCAATAAATACTGGTGGTGGCTCGACTTATTACGCCGAT
AGTGTCAAAGGACGATTTACTATAAGTTGGGACAACGCAAACAATACTTTGTTCCTCCAAAT
GAATAGCCTCAAATCAGAAGACACTGCCGTCTACTACTGTGCAGCAGAGGACTTTGGTAACT
CGAAAACTTACTGGGGCCAAGGGACTCTGGTCACTGTCTCT;
(SEQ ID NO: 73)
GACGTACAACTTGTGGAATCAGGTGGGGGTCTCGTTCAAGCTGGTGGGTCACTGCGGCTTTC
TTGCGCTGCAAGTGGGTTCCCTTATTTTTCCTGCTGTATGAGCTGGCATCGCCAAGCTCCTG
GAAAAGATCGGGAACTGGTCTCCAGTATTTCATCTGACGGGTCAACACATTACGCTGATAGC
GTCAGGGGGAGGTTTACTATTTCTAAAGGAAATGCAAGAAACACACTCTACCTCCAGATGAA
CAGTCTGAAGCCCGAAGACACCGCCGTGTATTTCTGTGCAGCTTCCCCTAGACAGCTCGGGC
CTTACTATGCTATGGACTACTGGGGTCAAGGAACCTCAGTCACCGTCTCC;
(SEQ ID NO: 74)
CAGCTTCAACTGGTAGAGAGTGGTGGAGGACTTGTTCAGCCTGGAGGGTCCCTTAGACTTAG
TTGCGCTGCCAGTGGGTTCACCCTGGACAACTACGCCATTGGCTGGTTTCGACAGGCCCCAG
GGAAAGAGCGAGAGGGGGTCTCTAGCATCAGTAGTTCCGGTGGTTCCACCAACTATGCTGAT
AGTGTGAAGGGGAGGTTTACTGTTTCTCGGGATAATGCAAAACATACCGTGTACCTGCAAAT
GAATAGCCTCAAACCAGAAGACACAGCCGTCTATTATTGCGCAGGAGTTGGGGACTACGGTA
GAAGGGCGATGGGTCAAGGCACCTCAGTCACCGTCTCC;
and
(SEQ ID NO: 75)
GAAGTTCAGCTTGTTGAATCAGGCGGCGGAAGTGTCCAAGCTGGCGAAACCCTTCGGCTCTC
CTGCACCGCATCCGGCTTCACATTCGATGATTCAGATATGGGCTGGTACAGGCAGGCTCCTG
GTGACGAATGTGAGCTGGTATCCACAATCTCCAGCGATGGCAGCACAAATTATGCTGACTCT
GTTAAAGGGCGATTTACCATCTCTCAAGATAACGCAAAGAACACCGTGTATCTGCAGATGAC
CTCATTGAAGCCAGAGGATACTGCCATATATTCATGCAGAACAATGGCCTCGAGGATTGGTT
GCTGGGGCCAAGGGACTCTGGTCACTGTCTCT.

10. The polynucleotide of claim 8, wherein the polynucleotide comprises a sequence that has at least 85% identity to a sequence selected from the group consisting of:
(SEQ ID NO: 80)
ATGCTCAGGCTGCTCTTGGCTCTCAACTTATTCCCTTCAATTCAAGTAACAGGAGGGCAAGT
TCAGCTTGTCGAGAGTGGTGGAGGATTGGTGCAGCCAGGCGGATCTCTCAGACTTTCCTGCG
CCGCCAGTGGTTCTATGTTTTCCATAAATGCAGTGGCCTGGTACAGGCAAGCACCAGGCGAG
CAAAGGGAACCGGTAGCCGCAATGACCTCTGGAGGTAGTACAATTTACGCTGATAGCGTCAA
GGGACGATTCACAATATCACGAGACAATGCCAAGAACACTGTCTACCTTCAAATGAGCAGTT
TGAAACCTGAGGATACAGCAGTATACTACTGCAATGGAGGATGGTCACTGGCTATGGACTAC
TGGGGTCAAGGAACCTCAGTCACCGTCTCCCCGACGCCAGCGCCAACTATTGCGAGTCAGCC
TCTCAGTCTGCGACCTGAGGCTTGTCGACCAGCAGCCGGAGGCGCAGTGCACACGAGGGGGC
TGGACTTCGCCTGTGATCCTTCTAAGCCCTTTTGGGTGCTGGTGGTGGTTGGTGGAGTCCTG
GCTTGCTATAGCTTGCTAGTAACAGTGGCCTTTATTATTTTCTGGGTGAGGAAACGGGGCAG
AAAGAAACTCCTGTATATATTCAAACAACCATTTATGAGACCAGTACAAACTACTCAAGAGG
AAGATGGCTGTAGCTGCCGATTTCCAGAAGAAGAAGAAGGAGGATGTGAACTGGCTAGCCTG
AGAGTGAAGTTCAGCAGGAGCGCAGACGCCCCCGCGTACCAGCAGGGCCAGAACCAGCTCTA
TAACGAGCTCAATCTAGGACGAAGAGAGGAGTACGATGTTTTGGACAAGAGACGTGGCCGGG
ACCCTGAGATGGGGGGAAAGCCGCAGAGAAGGAAGAACCCTCAGGAAGGCCTGTACAATGAA
CTGCAGAAAGATAAGATGGCGGAGGCCTACAGTGAGATTGGGATGAAAGGCGAGCGCCGGAG
GGGCAAGGGGCACGATGGCCTTTACCAGGGTCTCAGTACAGCCACCAAGGACACCTACGACG
CCCTTCACATGCAGGCCCTGCCCCCTCGCGCTAGC;
(SEQ ID NO: 81)
ATGCTCAGGCTGCTCTTGGCTCTCAACTTATTCCCTTCAATTCAAGTAACAGGAGGGCAGGT
TCAGCTGGTGGAGTCCGGCGGAGGTCTTGTTCAGCCCGGAGGTTCCCTGCGCCTCTCTTGTG
CAGCTTCTGGTTCCATTTTCTCAGTCAATGCAATGGGCTACTACAGACAAATTTCCACAAAG
CAGCGCAACTTGGTCGCCGCAATAAATACTGGTGGTGGCTCGACTTATTACGCCGATAGTGT
CAAAGGACGATTTACTATAAGTTGGGACAACGCAAACAATACTTTGTTCCTCCAAATGAATA
GCCTCAAATCAGAAGACACTGCCGTCTACTACTGTGCAGCAGAGGACTTTGGTAACTCGAAA
ACTTACTGGGGCCAAGGGACTCTGGTCACTGTCTCTCCGACGCCAGCGCCAACTATTGCGAG
TCAGCCTCTCAGTCTGCGACCTGAGGCTTGTCGACCAGCAGCCGGAGGCGCAGTGCACACGA
GGGGGCTGGACTTCGCCTGTGATCCTTCTAAGCCCTTTTGGGTGCTGGTGGTGGTTGGTGGA
GTCCTGGCTTGCTATAGCTTGCTAGTAACAGTGGCCTTTATTATTTTCTGGGTGAGGAAACG
GGGCAGAAAGAAACTCCTGTATATATTCAAACAACCATTTATGAGACCAGTACAAACTACTC
AAGAGGAAGATGGCTGTAGCTGCCGATTTCCAGAAGAAGAAGAAGGAGGATGTGAACTGGCT
AGCCTGAGAGTGAAGTTCAGCAGGAGCGCAGACGCCCCCGCGTACCAGCAGGGCCAGAACCA
GCTCTATAACGAGCTCAATCTAGGACGAAGAGAGGAGTACGATGTTTTGGACAAGAGACGTG
GCCGGGACCCTGAGATGGGGGGAAAGCCGCAGAGAAGGAAGAACCCTCAGGAAGGCCTGTAC
AATGAACTGCAGAAAGATAAGATGGCGGAGGCCTACAGTGAGATTGGGATGAAAGGCGAGCG
CCGGAGGGGCAAGGGGCACGATGGCCTTTACCAGGGTCTCAGTACAGCCACCAAGGACACCT
ACGACGCCCTTCACATGCAGGCCCTGCCCCCTCGCGCTAGC;
(SEQ ID NO: 82)
ATGCTCAGGCTGCTCTTGGCTCTCAACTTATTCCCTTCAATTCAAGTAACAGGAGGGGACGT
ACAACTTGTGGAATCAGGTGGGGGTCTCGTTCAAGCTGGTGGGTCACTGCGGCTTTCTTGCG
CTGCAAGTGGGTTCCCTTATTTTTCCTGCTGTATGAGCTGGCATCGCCAAGCTCCTGGAAAA
GATCGGGAACTGGTCTCCAGTATTTCATCTGACGGGTCAACACATTACGCTGATAGCGTCAG
GGGGAGGTTTACTATTTCTAAAGGAAATGCAAGAAACACACTCTACCTCCAGATGAACAGTC
TGAAGCCCGAAGACACCGCCGTGTATTTCTGTGCAGCTTCCCCTAGACAGCTCGGGCCTTAC
TATGCTATGGACTACTGGGGTCAAGGAACCTCAGTCACCGTCTCCCCGACGCCAGCGCCAAC
TATTGCGAGTCAGCCTCTCAGTCTGCGACCTGAGGCTTGTCGACCAGCAGCCGGAGGCGCAG
TGCACACGAGGGGGCTGGACTTCGCCTGTGATCCTTCTAAGCCCTTTTGGGTGCTGGTGGTG
GTTGGTGGAGTCCTGGCTTGCTATAGCTTGCTAGTAACAGTGGCCTTTATTATTTTCTGGGT
GAGGAAACGGGGCAGAAAGAAACTCCTGTATATATTCAAACAACCATTTATGAGACCAGTAC
AAACTACTCAAGAGGAAGATGGCTGTAGCTGCCGATTTCCAGAAGAAGAAGAAGGAGGATGT
GAACTGGCTAGCCTGAGAGTGAAGTTCAGCAGGAGCGCAGACGCCCCCGCGTACCAGCAGGG
CCAGAACCAGCTCTATAACGAGCTCAATCTAGGACGAAGAGAGGAGTACGATGTTTTGGACA
AGAGACGTGGCCGGGACCCTGAGATGGGGGGAAAGCCGCAGAGAAGGAAGAACCCTCAGGAA
GGCCTGTACAATGAACTGCAGAAAGATAAGATGGCGGAGGCCTACAGTGAGATTGGGATGAA
AGGCGAGCGCCGGAGGGGCAAGGGGCACGATGGCCTTTACCAGGGTCTCAGTACAGCCACCA
AGGACACCTACGACGCCCTTCACATGCAGGCCCTGCCCCCTCGCGCTAGC;
(SEQ ID NO: 83)
ATGCTCAGGCTGCTCTTGGCTCTCAACTTATTCCCTTCAATTCAAGTAACAGGAGGGCAGCT
TCAACTGGTAGAGAGTGGTGGAGGACTTGTTCAGCCTGGAGGGTCCCTTAGACTTAGTTGCG
CTGCCAGTGGGTTCACCCTGGACAACTACGCCATTGGCTGGTTTCGACAGGCCCCAGGGAAA
GAGCGAGAGGGGGTCTCTAGCATCAGTAGTTCCGGTGGTTCCACCAACTATGCTGATAGTGT
GAAGGGGAGGTTTACTGTTTCTCGGGATAATGCAAAACATACCGTGTACCTGCAAATGAATA
GCCTCAAACCAGAAGACACAGCCGTCTATTATTGCGCAGGAGTTGGGGACTACGGTAGAAGG
GCGATGGGTCAAGGCACCTCAGTCACCGTCTCCCCGACGCCAGCGCCAACTATTGCGAGTCA
GCCTCTCAGTCTGCGACCTGAGGCTTGTCGACCAGCAGCCGGAGGCGCAGTGCACACGAGGG
GGCTGGACTTCGCCTGTGATCCTTCTAAGCCCTTTTGGGTGCTGGTGGTGGTTGGTGGAGTC
CTGGCTTGCTATAGCTTGCTAGTAACAGTGGCCTTTATTATTTTCTGGGTGAGGAAACGGGG
CAGAAAGAAACTCCTGTATATATTCAAACAACCATTTATGAGACCAGTACAAACTACTCAAG
AGGAAGATGGCTGTAGCTGCCGATTTCCAGAAGAAGAAGAAGGAGGATGTGAACTGGCTAGC
CTGAGAGTGAAGTTCAGCAGGAGCGCAGACGCCCCCGCGTACCAGCAGGGCCAGAACCAGCT
CTATAACGAGCTCAATCTAGGACGAAGAGAGGAGTACGATGTTTTGGACAAGAGACGTGGCC
GGGACCCTGAGATGGGGGGAAAGCCGCAGAGAAGGAAGAACCCTCAGGAAGGCCTGTACAAT
GAACTGCAGAAAGATAAGATGGCGGAGGCCTACAGTGAGATTGGGATGAAAGGCGAGCGCCG
GAGGGGCAAGGGGCACGATGGCCTTTACCAGGGTCTCAGTACAGCCACCAAGGACACCTACG
ACGCCCTTCACATGCAGGCCCTGCCCCCTCGCGCTAGC;
(SEQ ID NO: 84)
ATGCTCAGGCTGCTCTTGGCTCTCAACTTATTCCCTTCAATTCAAGTAACAGGAGGGCAGCT
TCAACTGGTAGAGAGTGGTGGAGGACTTGTTCAGCCTGGAGGGTCCCTTAGACTTAGTTGCG
CTGCCAGTGGGTTCACCCTGGACTACTACGCCATTGGCTGGTTTCGACAGGCCCCAGGGAAA
GAGCGAGAGGGGGTCTCTTACATCAGTAGTTCCGGTGGTTCCACTAACTACGCTGATAGTGT
GAAGGGGAGGTTTACTATTTCTCGGGATAATGCAAAAAATACCGTGTACCTGCAAATGAATA
GCCTCAAACCAGAAGACACAGGCGTTTATTCTTGCGCAGCAGCTGGGGGCTATGGTAACTCC
TCTCGCTACTGGGGTCAAGGAACCTCAGTCACCGTCTCCCCGACGCCAGCGCCAACTATTGC
GAGTCAGCCTCTCAGTCTGCGACCTGAGGCTTGTCGACCAGCAGCCGGAGGCGCAGTGCACA
CGAGGGGGCTGGACTTCGCCTGTGATCCTTCTAAGCCCTTTTGGGTGCTGGTGGTGGTTGGT
GGAGTCCTGGCTTGCTATAGCTTGCTAGTAACAGTGGCCTTTATTATTTTCTGGGTGAGGAA
ACGGGGCAGAAAGAAACTCCTGTATATATTCAAACAACCATTTATGAGACCAGTACAAACTA
CTCAAGAGGAAGATGGCTGTAGCTGCCGATTTCCAGAAGAAGAAGAAGGAGGATGTGAACTG
GCTAGCCTGAGAGTGAAGTTCAGCAGGAGCGCAGACGCCCCCGCGTACCAGCAGGGCCAGAA
CCAGCTCTATAACGAGCTCAATCTAGGACGAAGAGAGGAGTACGATGTTTTGGACAAGAGAC
GTGGCCGGGACCCTGAGATGGGGGGAAAGCCGCAGAGAAGGAAGAACCCTCAGGAAGGCCTG
TACAATGAACTGCAGAAAGATAAGATGGCGGAGGCCTACAGTGAGATTGGGATGAAAGGCGA
GCGCCGGAGGGGCAAGGGGCACGATGGCCTTTACCAGGGTCTCAGTACAGCCACCAAGGACA
CCTACGACGCCCTTCACATGCAGGCCCTGCCCCCTCGCGCTAGC;
(SEQ ID NO: 85)
ATGCTCAGGCTGCTCTTGGCTCTCAACTTATTCCCTTCAATTCAAGTAACAGGAGGGGAAGT
TCAGCTTGTTGAATCAGGCGGCGGAAGTGTCCAAGCTGGCGAAACCCTTCGGCTCTCCTGCA
CCGCATCCGGCTTCACATTCGATGATTCAGATATGGGCTGGTACAGGCAGGCTCCTGGTGAC
GAATGTGAGCTGGTATCCACAATCTCCAGCGATGGCAGCACAAATTATGCTGACTCTGTTAA
AGGGCGATTTACCATCTCTCAAGATAACGCAAAGAACACCGTGTATCTGCAGATGACCTCAT
TGAAGCCAGAGGATACTGCCATATATTCATGCAGAACAATGGCCTCGAGGATTGGTTGCTGG
GGCCAAGGGACTCTGGTCACTGTCTCTCCGACGCCAGCGCCAACTATTGCGAGTCAGCCTCT
CAGTCTGCGACCTGAGGCTTGTCGACCAGCAGCCGGAGGCGCAGTGCACACGAGGGGGCTGG
ACTTCGCCTGTGATCCTTCTAAGCCCTTTTGGGTGCTGGTGGTGGTTGGTGGAGTCCTGGCT
TGCTATAGCTTGCTAGTAACAGTGGCCTTTATTATTTTCTGGGTGAGGAAACGGGGCAGAAA
GAAACTCCTGTATATATTCAAACAACCATTTATGAGACCAGTACAAACTACTCAAGAGGAAG
ATGGCTGTAGCTGCCGATTTCCAGAAGAAGAAGAAGGAGGATGTGAACTGGCTAGCCTGAGA
GTGAAGTTCAGCAGGAGCGCAGACGCCCCCGCGTACCAGCAGGGCCAGAACCAGCTCTATAA
CGAGCTCAATCTAGGACGAAGAGAGGAGTACGATGTTTTGGACAAGAGACGTGGCCGGGACC
CTGAGATGGGGGGAAAGCCGCAGAGAAGGAAGAACCCTCAGGAAGGCCTGTACAATGAACTG
CAGAAAGATAAGATGGCGGAGGCCTACAGTGAGATTGGGATGAAAGGCGAGCGCCGGAGGGG
CAAGGGGCACGATGGCCTTTACCAGGGTCTCAGTACAGCCACCAAGGACACCTACGACGCCC
TTCACATGCAGGCCCTGCCCCCTCGCGCTAGC;
and
(SEQ ID NO: 86)
ATGCTCAGGCTGCTCTTGGCTCTCAACTTATTCCCTTCAATTCAAGTAACAGGAGGGCAAGT
TCAGCTTGTGGAGTCAGGCGGTGGACTCGTTCAAGCCGGAGGCTCACTGCGGTTGTCTTGTG
TCGCATCTGGTCGAACCTTCTCAAGTTATGCTATGGGGTGGTTCCGCCAGTCTCCTGGTAAA
GAGCGGGAATTTGTCGCAGCAATTTGTTGGTCTGGTGGTAACACTTACTATGCTGATTCAGT
AAAAGGTCGATTCACCATATCAGGGGATAATGCAAAAAATTCTGTCTATCTCCAAATGAACT
CTCTGAAGCCTGAAGATACTGCTGTGTATATCTGTAATGAGAGGACTACGGTACTACCTACG
GGTGCTATGGACTACTGGGGTCAAGGGACCTCAGTCACCGTCTCCTCGCCGACGCCAGCGCC
AACTATTGCGAGTCAGCCTCTCAGTCTGCGACCTGAGGCTTGTCGACCAGCAGCCGGAGGCG
CAGTGCACACGAGGGGGCTGGACTTCGCCTGTGATCCTTCTAAGCCCTTTTGGGTGCTGGTG
GTGGTTGGTGGAGTCCTGGCTTGCTATAGCTTGCTAGTAACAGTGGCCTTTATTATTTTCTG
GGTGAGGAAACGGGGCAGAAAGAAACTCCTGTATATATTCAAACAACCATTTATGAGACCAG
TACAAACTACTCAAGAGGAAGATGGCTGTAGCTGCCGATTTCCAGAAGAAGAAGAAGGAGGA
TGTGAACTGGCTAGCCTGAGAGTGAAGTTCAGCAGGAGCGCAGACGCCCCCGCGTACCAGCA
GGGCCAGAACCAGCTCTATAACGAGCTCAATCTAGGACGAAGAGAGGAGTACGATGTTTTGG
ACAAGAGACGTGGCCGGGACCCTGAGATGGGGGGAAAGCCGCAGAGAAGGAAGAACCCTCAG
GAAGGCCTGTACAATGAACTGCAGAAAGATAAGATGGCGGAGGCCTACAGTGAGATTGGGAT
GAAAGGCGAGCGCCGGAGGGGCAAGGGGCACGATGGCCTTTACCAGGGTCTCAGTACAGCCA
CCAAGGACACCTACGACGCCCTTCACATGCAGGCCCTGCCCCCTCGCGCTAGC.

11. A vector comprising the polynucleotide of claim 8.

12. A cell comprising the polynucleotide of claim 8.

13. A pharmaceutical composition comprising an effective amount of the polypeptide of claim 1, and a pharmaceutically acceptable excipient, carrier, or diluent.

14. A method for treating a subject having a neoplasia, the method comprising administering to the subject the pharmaceutical composition of claim 13.

15. A kit comprising the polypeptide of claim 1, and a container, for use in treating a subject having a neoplasia.

16. A method of detecting mesothelin or a fragment thereof in a sample, the method comprising:
contacting the sample with at least one detectably labeled VHH binding protein or an antigen binding fragment thereof that specifically binds to mesothelin or a peptide thereof, wherein the binding protein or the antigen binding fragment thereof comprises three Complementarity Determining Regions (CDRs), CDR1, CDR2, and CDR3 structurally positioned between four framework (FR) regions (FR1-FR4) as follows: FR1-CDR1-FR2-CDR2-FR3-CDR3-FR4; wherein the three CDRs are selected from:
CDR1 comprising the amino acid sequence GRTFSSYA (SEQ ID NO: 1); CDR2 comprising the amino acid sequence ICWSGGNT (SEQ ID NO: 2); and CDR3 comprising the amino acid sequence NERTTVLPTGAMDY (SEQ ID NO: 3);
CDR1 comprising the amino acid sequence GSMFSINA (SEQ ID NO: 4); CDR2 comprising the amino acid sequence MTSGGST (SEQ ID NO: 5); and CDR3 comprising the amino acid sequence NGGWSLAMDY (SEQ ID NO: 6);
CDR1 comprising the amino acid sequence GFTLDYYA (SEQ ID NO: 7); CDR2 comprising the amino acid sequence ISSSGGST (SEQ ID NO: 8); and CDR3 comprising the amino acid sequence AAAGGYGNSSRY (SEQ ID NO: 9);
CDR1 comprising the amino acid sequence GSIFSVNA (SEQ ID NO: 10); CDR2 comprising the amino acid sequence INTGGGST (SEQ ID NO: 11); and CDR3 comprising the amino acid sequence AAEDFGNSKTY (SEQ ID NO: 12);
CDR1 comprising the amino acid sequence GFPYFSCC (SEQ ID NO: 13); CDR2 comprising the amino acid sequence ISSDGST (SEQ ID NO: 14); and CDR3 comprising the amino acid sequence AASPRQLGPYYAMDY (SEQ ID NO: 15);
CDR1 comprising the amino acid sequence GFTLDNYA (SEQ ID NO: 16); CDR2 comprising the amino acid sequence ISSSGGST (SEQ ID NO: 8); and CDR3 comprising the amino acid sequence AGVGDYGRRA (SEQ ID NO: 17); and
CDR1 comprising the amino acid sequence GFTFDDSD (SEQ ID NO: 18); CDR2 comprising the amino acid sequence ISSDGST (SEQ ID NO: 14); and CDR3 comprising the amino acid sequence RTMASRIGC (SEQ ID NO: 19); and wherein
i) FR1 comprises the following amino acid sequence:
X₁X₂QLVESGGGX₃VQX₄GX₅X₆LRLX₇CX₈AS (SEQ ID NO: 20), wherein
X₁ is E, D, or Q,
X₂ is V or L,
X₃ is L or S,
X₄ is A or P,
X₅ is E or G,
X₆ is S or T,
X₇ is S or T, and
X₈ is A, T, or V;
ii) FR2 comprises the following amino acid sequence:
XₐX_{b}X₁₃X₁₄RQX₁₅X₁₆X₁₇X₁₈X₁₉X₂₀X₂₁X₂₂VX₂₃X_{c}, wherein
Xₐ is I, M, or V,
X_{b} is A, G, or S,
X₁₃ is W or Y,
X₁₄ is F, H, or Y,
X₁₅ is A, I, or X,
X₁₆ is P or S,
X₁₇ is G or T,
X₁₈ is D, K, or E,
X₁₉ is D, E, or Q,
X₂₀ is C or R,
X₂₁ is E or N,
X₂₂ is F, G, L, or P,
X₂₃ is S or A, and
X_{c} is A, S, T, or Y;
iii) FR3 comprises the following amino acid sequence:
X_{d}YAXₑX_{f}VX_{g}GRFTX₂₄SX₂₅X₂₆X₂₇AX₂₈X₂₉X₃₀X₃₁X₃₂LQMX₃₃SLKX₃₄EDT X₃₅X₃₆YX₃₇C (SEQ ID NO: 21), wherein
X_{d} is F, H, I, N, or Y,
Xₑ is A or D,
X_{f} is S or T,
X_{g} is K or R,
X₂₄ is I or V,
X₂₅ is G, K, R, Q, or W,
X₂₆ is D or G,
X₂₇ is N or T,
X₂₈ is K, N, or R,
X₂₉ is N, H, or Q,
X₃₀ is T or S,
X₃₁ is V or L,
X₃₂ is F or Y,
X₃₃ is N, S, or T,
X₃₄ is P or S,
X₃₅ is A or G,
X₃₆ is I or V, and
X₃₇ is F, I, S, or Y; and
iv) FR4 comprises the following amino acid sequence:
X₄₀GQGTX₄₁VTVX₄₂X₄₃ (SEQ ID NO: 22), wherein
X₄₀ is M or W,
X₄₁ is L or S,
X₄₂ is null or S, and
X₄₃ is null or S; and
measuring the level of binding of the binding protein to mesothelin in the sample relative to a control to detect or identify the presence of mesothelin in the sample.

17. A chimeric antigen receptor (CAR) comprising an amino acid sequence with at least about 95% identity to one of the following sequences:
(SEQ ID NO: 23)
MLRLLLALNLFPSIQVTGGQVQLVESGGGLVQPGGSLRLSCAASGSMFSINAVAWYRQAPGE
QREPVAAMTSGGSTIYADSVKGRFTISRDNAKNTVYLQMSSLKPEDTAVYYCNGGWSLAMDY
WGQGTSVTVSPTPAPTIASQPLSLRPEACRPAAGGAVHTRGLDFACDPSKPFWVLVVVGGVL
ACYSLLVTVAFIIFWVRKRGRKKLLYIFKQPFMRPVQTTQEEDGCSCRFPEEEEGGCELASL
RVKFSRSADAPAYQQGQNQLYNELNLGRREEYDVLDKRRGRDPEMGGKPQRRKNPQEGLYNE
LQKDKMAEAYSEIGMKGERRRGKGHDGLYQGLSTATKDTYDALHMQALPPRAS;
(SEQ ID NO: 24)
MLRLLLALNLFPSIQVTGGQVQLVESGGGLVQPGGSLRLSCAASGSIFSVNAMGYYRQISTK
QRNLVAAINTGGGSTYYADSVKGRFTISWDNANNTLFLQMNSLKSEDTAVYYCAAEDFGNSK
TYWGQGTLVTVSPTPAPTIASQPLSLRPEACRPAAGGAVHTRGLDFACDPSKPFWVLVVVGG
VLACYSLLVTVAFIIFWVRKRGRKKLLYIFKQPFMRPVQTTQEEDGCSCRFPEEEEGGCELA
SLRVKFSRSADAPAYQQGQNQLYNELNLGRREEYDVLDKRRGRDPEMGGKPQRRKNPQEGLY
NELQKDKMAEAYSEIGMKGERRRGKGHDGLYQGLSTATKDTYDALHMQALPPRAS;
(SEQ ID NO: 25)
MLRLLLALNLFPSIQVTGGDVQLVESGGGLVQAGGSLRLSCAASGFPYFSCCMSWHRQAPGK
DRELVSSISSDGSTHYADSVRGRFTISKGNARNTLYLQMNSLKPEDTAVYFCAASPRQLGPY
YAMDYWGQGTSVTVSPTPAPTIASQPLSLRPEACRPAAGGAVHTRGLDFACDPSKPFWVLVV
VGGVLACYSLLVTVAFIIFWVRKRGRKKLLYIFKQPFMRPVQTTQEEDGCSCRFPEEEEGGC
ELASLRVKFSRSADAPAYQQGQNQLYNELNLGRREEYDVLDKRRGRDPEMGGKPQRRKNPQE
GLYNELQKDKMAEAYSEIGMKGERRRGKGHDGLYQGLSTATKDTYDALHMQALPPRAS;
(SEQ ID NO: 26)
MLRLLLALNLFPSIQVTGGQLQLVESGGGLVQPGGSLRLSCAASGFTLDNYAIGWFRQAPGK
EREGVSSISSSGGSTNYADSVKGRFTVSRDNAKHTVYLQMNSLKPEDTAVYYCAGVGDYGRR
AMGQGTSVTVSPTPAPTIASQPLSLRPEACRPAAGGAVHTRGLDFACDPSKPFWVLVVVGGV
LACYSLLVTVAFIIFWVRKRGRKKLLYIFKQPFMRPVQTTQEEDGCSCRFPEEEEGGCELAS
LRVKFSRSADAPAYQQGQNQLYNELNLGRREEYDVLDKRRGRDPEMGGKPQRRKNPQEGLYN
ELQKDKMAEAYSEIGMKGERRRGKGHDGLYQGLSTATKDTYDALHMQALPPRAS;
(SEQ ID NO: 27)
MLRLLLALNLFPSIQVTGGQLQLVESGGGLVQPGGSLRLSCAASGFTLDYYAIGWFRQAPGK
EREGVSYISSSGGSTNYADSVKGRFTISRDNAKNTVYLQMNSLKPEDTGVYSCAAAGGYGNS
SRYWGQGTSVTVSPTPAPTIASQPLSLRPEACRPAAGGAVHTRGLDFACDPSKPFWVLVVVG
GVLACYSLLVTVAFIIFWVRKRGRKKLLYIFKQPFMRPVQTTQEEDGCSCRFPEEEEGGCEL
ASLRVKFSRSADAPAYQQGQNQLYNELNLGRREEYDVLDKRRGRDPEMGGKPQRRKNPQEGL
YNELQKDKMAEAYSEIGMKGERRRGKGHDGLYQGLSTATKDTYDALHMQALPPRAS;
(SEQ ID NO: 28)
MLRLLLALNLFPSIQVTGGEVQLVESGGGSVQAGETLRLSCTASGFTFDDSDMGWYRQAPGD
ECELVSTISSDGSTNYADSVKGRFTISQDNAKNTVYLQMTSLKPEDTAIYSCRTMASRIGCW
GQGTLVTVSPTPAPTIASQPLSLRPEACRPAAGGAVHTRGLDFACDPSKPFWVLVVVGGVLA
CYSLLVTVAFIIFWVRKRGRKKLLYIFKQPFMRPVQTTQEEDGCSCRFPEEEEGGCELASLR
VKFSRSADAPAYQQGQNQLYNELNLGRREEYDVLDKRRGRDPEMGGKPQRRKNPQEGLYNEL
QKDKMAEAYSEIGMKGERRRGKGHDGLYQGLSTATKDTYDALHMQALPPRAS;
and
(SEQ ID NO: 29)
MLRLLLALNLFPSIQVTGGQVQLVESGGGLVQAGGSLRLSCVASGRTFSSYAMGWFRQSPGK
EREFVAAICWSGGNTYYADSVKGRFTISGDNAKNSVYLQMNSLKPEDTAVYICNERTTVLPT
GAMDYWGQGTSVTVSSPTPAPTIASQPLSLRPEACRPAAGGAVHTRGLDFACDPSKPFWVLV
VVGGVLACYSLLVTVAFIIFWVRKRGRKKLLYIFKQPFMRPVQTTQEEDGCSCRFPEEEEGG
CELASLRVKFSRSADAPAYQQGQNQLYNELNLGRREEYDVLDKRRGRDPEMGGKPQRRKNPQ
EGLYNELQKDKMAEAYSEIGMKGERRRGKGHDGLYQGLSTATKDTYDALHMQALPPRAS.

18. A T cell expressing the CAR of claim 17 or a polynucleotide encoding the CAR.

19. A VH-heavy chain only (VHH) polypeptide or an antigen binding portion thereof that specifically binds to mesothelin, wherein the binding protein or the antigen binding portion thereof comprises an amino acid sequence with at least about 95% identity to one of the following sequences:
(SEQ ID NO: 23)
MLRLLLALNLFPSIQVTGGQVQLVESGGGLVQPGGSLRLSCAASGSMFSINAVAWYRQAPGE
QREPVAAMTSGGSTIYADSVKGRFTISRDNAKNTVYLQMSSLKPEDTAVYYCNGGWSLAMDY
WGQGTSVTVSPTPAPTIASQPLSLRPEACRPAAGGAVHTRGLDFACDPSKPFWVLVVVGGVL
ACYSLLVTVAFIIFWVRKRGRKKLLYIFKQPFMRPVQTTQEEDGCSCRFPEEEEGGCELASL
RVKFSRSADAPAYQQGQNQLYNELNLGRREEYDVLDKRRGRDPEMGGKPQRRKNPQEGLYNE
LQKDKMAEAYSEIGMKGERRRGKGHDGLYQGLSTATKDTYDALHMQALPPRAS;
(SEQ ID NO: 24)
MLRLLLALNLFPSIQVTGGQVQLVESGGGLVQPGGSLRLSCAASGSIFSVNAMGYYRQISTK
QRNLVAAINTGGGSTYYADSVKGRFTISWDNANNTLFLQMNSLKSEDTAVYYCAAEDFGNSK
TYWGQGTLVTVSPTPAPTIASQPLSLRPEACRPAAGGAVHTRGLDFACDPSKPFWVLVVVGG
VLACYSLLVTVAFIIFWVRKRGRKKLLYIFKQPFMRPVQTTQEEDGCSCRFPEEEEGGCELA
SLRVKFSRSADAPAYQQGQNQLYNELNLGRREEYDVLDKRRGRDPEMGGKPQRRKNPQEGLY
NELQKDKMAEAYSEIGMKGERRRGKGHDGLYQGLSTATKDTYDALHMQALPPRAS;
(SEQ ID NO: 25)
MLRLLLALNLFPSIQVTGGDVQLVESGGGLVQAGGSLRLSCAASGFPYFSCCMSWHRQAPGK
DRELVSSISSDGSTHYADSVRGRFTISKGNARNTLYLQMNSLKPEDTAVYFCAASPRQLGPY
YAMDYWGQGTSVTVSPTPAPTIASQPLSLRPEACRPAAGGAVHTRGLDFACDPSKPFWVLVV
VGGVLACYSLLVTVAFIIFWVRKRGRKKLLYIFKQPFMRPVQTTQEEDGCSCRFPEEEEGGC
ELASLRVKFSRSADAPAYQQGQNQLYNELNLGRREEYDVLDKRRGRDPEMGGKPQRRKNPQE
GLYNELQKDKMAEAYSEIGMKGERRRGKGHDGLYQGLSTATKDTYDALHMQALPPRAS;
(SEQ ID NO: 26)
MLRLLLALNLFPSIQVTGGQLQLVESGGGLVQPGGSLRLSCAASGFTLDNYAIGWFRQAPGK
EREGVSSISSSGGSTNYADSVKGRFTVSRDNAKHTVYLQMNSLKPEDTAVYYCAGVGDYGRR
AMGQGTSVTVSPTPAPTIASQPLSLRPEACRPAAGGAVHTRGLDFACDPSKPFWVLVVVGGV
LACYSLLVTVAFIIFWVRKRGRKKLLYIFKQPFMRPVQTTQEEDGCSCRFPEEEEGGCELAS
LRVKFSRSADAPAYQQGQNQLYNELNLGRREEYDVLDKRRGRDPEMGGKPQRRKNPQEGLYN
ELQKDKMAEAYSEIGMKGERRRGKGHDGLYQGLSTATKDTYDALHMQALPPRAS;
(SEQ ID NO: 27)
MLRLLLALNLFPSIQVTGGQLQLVESGGGLVQPGGSLRLSCAASGFTLDYYAIGWFRQAPGK
EREGVSYISSSGGSTNYADSVKGRFTISRDNAKNTVYLQMNSLKPEDTGVYSCAAAGGYGNS
SRYWGQGTSVTVSPTPAPTIASQPLSLRPEACRPAAGGAVHTRGLDFACDPSKPFWVLVVVG
GVLACYSLLVTVAFIIFWVRKRGRKKLLYIFKQPFMRPVQTTQEEDGCSCRFPEEEEGGCEL
ASLRVKFSRSADAPAYQQGQNQLYNELNLGRREEYDVLDKRRGRDPEMGGKPQRRKNPQEGL
YNELQKDKMAEAYSEIGMKGERRRGKGHDGLYQGLSTATKDTYDALHMQALPPRAS;
(SEQ ID NO: 28)
MLRLLLALNLFPSIQVTGGEVQLVESGGGSVQAGETLRLSCTASGFTFDDSDMGWYRQAPGD
ECELVSTISSDGSTNYADSVKGRFTISQDNAKNTVYLQMTSLKPEDTAIYSCRTMASRIGCW
GQGTLVTVSPTPAPTIASQPLSLRPEACRPAAGGAVHTRGLDFACDPSKPFWVLVVVGGVLA
CYSLLVTVAFIIFWVRKRGRKKLLYIFKQPFMRPVQTTQEEDGCSCRFPEEEEGGCELASLR
VKFSRSADAPAYQQGQNQLYNELNLGRREEYDVLDKRRGRDPEMGGKPQRRKNPQEGLYNEL
QKDKMAEAYSEIGMKGERRRGKGHDGLYQGLSTATKDTYDALHMQALPPRAS;
and
(SEQ ID NO: 29)
MLRLLLALNLFPSIQVTGGQVQLVESGGGLVQAGGSLRLSCVASGRTFSSYAMGWFRQSPGK
EREFVAAICWSGGNTYYADSVKGRFTISGDNAKNSVYLQMNSLKPEDTAVYICNERTTVLPT
GAMDYWGQGTSVTVSSPTPAPTIASQPLSLRPEACRPAAGGAVHTRGLDFACDPSKPFWVLV
VVGGVLACYSLLVTVAFIIFWVRKRGRKKLLYIFKQPFMRPVQTTQEEDGCSCRFPEEEEGG
CELASLRVKFSRSADAPAYQQGQNQLYNELNLGRREEYDVLDKRRGRDPEMGGKPQRRKNPQ
EGLYNELQKDKMAEAYSEIGMKGERRRGKGHDGLYQGLSTATKDTYDALHMQALPPRAS.
or a polynucleotide encoding the polypeptide.

20. A vector comprising the polynucleotide of claim 19.

21. A VH-heavy chain only (VHH) polypeptide comprising an amino acid sequence having at least 85% sequence identity to a full-length amino acid sequence of any one of Tables 1, 8A to 8G, or 10A to 10M, or a functional fragment thereof, and/or comprising a set of Complementarity Determining Regions: CDR1, CDR2, and CDR3, listed in a row of any one of Tables 3A, 8A to 8G, 10A to 10M, or 11 or a polynucleotide encoding the polypeptide.
